(19) United States
(12) Patent Application Publication
(10) Pub. No.: US 2021/0205475 A1
(43) Pub. Date: Jul. 8, 2021

(54) MITOCHONDRIAL OPTOGENETICS-BASED GENE THERAPY FOR TREATING CANCER

(71) Applicant: THE UAB RESEARCH FOUNDATION, Birmingham, AL (US)

(72) Inventors: Xiaoguang Liu, Vestavia Hills, AL (US); Lufang Zhou, Vestavia Hills, AL (US); Jianyi Zhang, Vestavia Hills, AL (US); Patrick James Ernst, Birmingham, AL (US); Ningning Xu, Acton, MA (US)

(21) Appl. No.: 17/055,812

(22) PCT Filed: May 17, 2019
(86) PCT No.: PCT/US2019/032772

§ 371 Date: Nov. 16, 2020

Related U.S. Application Data
(60) Provisional application No. 62/672,749, filed on May 17, 2018.

(51) Int. Cl. A61K 48/00; C07K 14/405

(52) U.S. Cl. CPC A61K 48/005; C07K 2319/61; C07K 2319/07; C07K 14/405

(57) ABSTRACT

Disclosed herein is an optogenetics-based gene therapy that involves channelrhodopsin fusion proteins having an inner mitochondrial membrane-mitochondrial localization signal (IMM-MLS) that can effectively target the fusion protein to an inner mitochondria membrane, and a channelrhodopsin ion channel domain that can change the mitochondrial membrane potential (ΔΨm) when light is present. The disclosed optogenetics-based gene therapy system can in some embodiments further involve luciferase fusion proteins to stimulate the channelrhodopsin without reliance on external light that has an outer mitochondrial membrane-mitochondrial localization signal (OMM-MLS) that can effectively target the luciferase fusion protein to an outer mitochondrial membrane, and a luciferase protein that can produce a bioluminescence in the presence of a luciferase substrate.

### CROSS-REFERENCE TO RELATED APPLICATIONS

This application claims benefit of U.S. Provisional Application No. 62/672,749, filed May 17, 2018, which is hereby incorporated herein by reference in its entirety.

### STATEMENT REGARDING FEDERALLY SPONSORED RESEARCH OR DEVELOPMENT

This invention was made with Government Support under Grant No. HL127599 awarded by the National Institutes of Health. The Government has certain rights in the invention.

### SEQUENCE LISTING

This application contains a sequence listing filed in electronic form as an ASCII.txt file entitled 222104_2840_Sequence_Listing_ST25” created on May 15, 2019. The content of the sequence listing is incorporated herein in its entirety.

### BACKGROUND

Various therapies, such as chemotherapy, radiotherapy, antibody-based therapy and immunotherapy, have been developed to treat cancers. These therapies are limited by chemoresistance, severe side effects, or the low efficiency to treat recurring or heterogeneous cancers. Targeted therapies can treat certain cancers that demonstrate the same phenotype, such as surface receptor, but the cancers coverage rate is still limited. Therefore, it is highly desired to develop a universal cancer treatment strategy that can treat multiple cancers.

NET Neuroendocrine (NE) cancers such as carcinoid, pancreatic islet cell tumors, and medullary thyroid cancer frequently metastasize to the liver (Adler, J. T., et al. Oncologist 2008 13:779-793; Pinchot, S. N., et al. 2008 Curr Opin Investig Drugs 9:576-582; Chen, H., et al. 1998 J Am Coll Surg 187:88-92; Chen, H., et al. 1998 J Gastrointest Surg 2:151-155; Chen, H. 2008 J Surg Oncol 97:203-204). They are the second most prevalent GI malignancy (Yao, J. C., et al. 2008 J Clin Oncol 26:3063-3072). Ninety percent of patients with pancreatic carcinoid tumors and 50% of patients with islet cell tumors develop isolated hepatic metastases (Hiller, N., et al. 1998 Abdom Imaging 23:188-190; Brown, K. T., et al. 1999 J Vasc Intery Radiol 10:397-403; Pinchot, S. N., et al. 2008 Oncologist 13:1255-1269; Isozaki, T., et al. 1999 Intern Med 38:17-21). Patients with untreated, isolated NE liver metastases have a<30% 5-year survival probability. It is reported that there are in excess of 100,000 patients living with NE cancers in US, 16,000 new diagnoses each year, and estimated more than 200,000 undiagnosed cases (Chen, H., et al. 1998 J Am Coll Surg 187:88-92; Norton, J. A. 2005 Best Pract Res Clin Gastroenterol 19:577-583). Thus, it is highly desired to develop new therapies to treat NE cancers.

The surgical resection alone is often curative in early-stage disease with localized tumors, but 40-95% of NE cancer patients are metastatic at the time of initial diagnosis (Shiba, S., et al. 2016 Pancreatology 16:99-105), and the widespread metastases at presentation make complete resections impossible. Considering the degree of hepatic involvement by the NE cancers, many patients are not candidates for operative intervention and the NE cancer resection is often followed by recurrence within the surgical bed. Moreover, other forms of therapy, including chemoembolization, radioembolization, radio-frequency ablation, cryoablation and chemotherapy (i.e. the mTOR inhibitor “everolimus” and multikinase inhibitor “sunitinib”), showed limited efficacy and caused severe systemic toxicities (Brown, K. T., et al. 1999 J Vasc Intery Radiol 10:397-403; Isozaki, T., et al. 1999 Intern Med 38:17-21; Eriksson, B., et al. 2008 Neuroendocrinology 87:8-19; Lal, A. & Chen, H. 2006 Curr Opin Oncol 18:9-15; Lehnert, T. 1998 Transplantation 66:1307-1312; Zhang, R., et al. 1999 Endocrinology 140:2152-2158; Boudreaux, J. P., et al. 2005 Ann Surg 241:839-845; Nguyen, C., et al. 2004 J Nucl Med 45:1660-1668; Fiorentini, G., et al. 2004 J Chemother 16:293-297; Zuetenhorst, J. M., et al. 2004 Endocr Relat Cancer 11:553-561). Therefore, besides surgery, there are no curative treatments for NE cancers and their metastases. However, even hepatic resection is often followed by recurrence within the surgical bed. Furthermore, patients with liver metastases from NE cancers often have debilitating symptoms, such as uncontrollable diarrhea, flushing, skin rashes, and heart failure, due to the excessive hormone secretion that characterizes these tumors (Brown, K. T., et al. 1999 J Vasc Intery Radiol 10:397-403; Miller, C.A. & Ellison, E. C. 1998 Surg Oncol Clin N Am 7:863-879). Thus, NE cancer patients frequently have a poor quality of life, emphasizing the critical need for the development of new therapeutic strategies to reduce the progression of NE malignancies.

BC (TNBC) In patients with breast cancer (BC), endocrine therapy to target the estrogen receptor, progesterone receptor, or human epidermal growth factor receptor is an effective approach after surgery, but strategies to target triple-negative BC (TNBC) are still needed. TNBC accounts for 10-20% of all BCs and is characterized by rapid growth, metastasis, and recurrence (Foulkes, W. D., et al. 2010 N Engl J Med 363:1938-1948). Unfortunately, the severe adverse effects and drug resistance associated with standard cytotoxic chemotherapies (e.g., doxorubicin, paclitaxel, and gemcitabine (GC)) minimize the clinical benefit of these drugs in patients with TNBC (Hung, S. W., et al. 2012 Cancer Lett 320:138-149). Targeted therapies, such as monoclonal antibodies, antibody-drug conjugates, chimeric antigen receptor engineered T cells, and small molecule inhibitors, have been developed to treat various solid tumors while minimizing the adverse effects on normal cells (Zhou, et al. 2014 Cancer Lett 352:145-151; Almasbak, H., et al. 2016 J Immunol Res 2016:5474602; Dai, H., et al. 2016 J Natl Cancer Inst 108; Magee, M.S. & Snook, A. E. 2014 Discov Med 18:265-271; Zhang, B., et al. 2016 Sci China Life Sci 59:340-348; Kunert, R. & Reinhart, D. 2016 Appl Microbiol Biotechnol 100:3451-3461; Polakis, P. 2016 Pharmacol Rev 68:3-19), but none of these therapies has been shown to effectively treat TNBC.

Despite the advances in research, diagnosis, and treatment, glioblastoma multiforme (GBM) remains one of the most common and deadliest form of brain tumors, with the 5-year survival rate less than 5% (Ostrom Q T, et al. Neuro Oncol. 2016 18(suppl_5):v1-v75). Currently used surgical debulking, chemotherapeutic agents (e.g. temozolomide, or TMZ), and radiotherapy strategies can only slightly extend the life expectancies of GBM patients. The main obstacle to successful GBM treatment lies both in its inherent complexity (e.g., recurrence and heterogeneity) and numerous mechanisms of TMZ resistance. Thus, improving therapeutic efficacy and overcoming drug resistance are the major goals of today's anti-cancer therapy development (Allinen M, et al. Cancer Cell. 2004 6(1):17-32; Stupp R, et al. N Engl J Med. 2005 352(10):987-96; Furnari F B, et al. Genes Dev. 2007 21(21):2683-710; Maher E A, et al. Genes Dev. 2001 15(11):1311-33).

While chemoresistance is the most challenging problem in GBM treatment and the major reason of chemotherapy failure, the underlying mechanisms remain incompletely understood. In addition, GBM is characterized by a marked heterogeneity at the cellular and molecular levels, which is another significant challenge for successful treatment (Friedmann-Morvinski D. Crit Rev Oncog. 2014 19(5):327-36). Heterogeneity confounds the design of effective therapies, as it will be unlikely to suppress GBM by targeting a single gene or a single pathway that can undergo unpredictable mutations.

Mitochondria play central roles in multiple cellular processes such as energy production and cell death (Honda H M, et al. Ann N Y Acad Sci. 2005 1047:248-58). While the precise steps whereby mitochondria provoke the transition of cells to the commitment of cell death are not well understood, a profound depolarization of Δψₘ is the crucial event and is considered as a point of no return. Because of the important role of mitochondria in cell death and recognized mitochondrial abnormalities in cancer cells, the mitochondrion has emerged as a promising target in cancer treatment. Directly targeting mitochondria offers the advantage to initiate cell death independent of upstream signal transduction elements that are frequently impaired in cancers, thus bypassing some mechanisms of proliferation and apoptosis-resistance.

### SUMMARY

A mitochondrial optogenetics-based gene therapy was developed and evaluated for anti-cancer toxicity or efficacy for the treatment of neuroendocrine tumors (NET), breast cancers (BC) including the triple negative breast cancer (TNBC), and glioblastoma multiforme (GBM).

In particular, disclosed herein is an optogenetics-based gene therapy that involves channelrhodopsin fusion proteins, nucleic acids encoding the fusion proteins, and vector systems for expressing these nucleic acids and proteins in cancer cells. The channelrhodopsin fusion protein has at least two domains: 1) a channelrhodopsin ion channel domain that can change the mitochondrial membrane potential (Δψm) when light is present, 2) an inner mitochondrial membrane-mitochondrial localization signal (IMM-MLS) that can effectively target the fusion protein to an inner mitochondria membrane.

The disclosed optogenetics-based gene therapy system can in some embodiments further involve luciferase fusion proteins to stimulate the channelrhodopsin without reliance on external light, as well as nucleic acids encoding the fusion proteins, and vector systems for co-expressing these nucleic acids and proteins in cancer cells with the channelrhodopsin fusion proteins. The luciferase fusion protein has at least two domains: 1) an outer mitochondrial membrane-mitochondrial leading signal (OMM-MLS) that can effectively target the luciferase fusion protein to an outer mitochondrial membrane, and 2) a luciferase protein that can produce a bioluminescence in the presence of a luciferase substrate.

Therefore, disclosed herein is a fusion protein that includes a Chloromonas oogama channelrhodopsin (CoChR) photoreceptor linked to an inner mitochondrial membrane-mitochondrial localization signal (IMM-MLS). The IMM-MLS can therefore be a leading sequence from a mitochondrial inner membrane protein. Examples of IMM-MLS include ABCB140, ABCB10(105), Cytochrome C MLS (mito), and renal outer medullary potassium channel (ROMK) MLS.

In some embodiments, the CoChR photoreceptor has an amino acid having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:1.

Also disclosed herein is an expression vector that includes a nucleic acid sequence encoding a channelrhodopsin fusion protein operably linked to an expression control sequence and a nucleic acid sequence encoding a luciferase fusion protein operably linked to an expression control sequence. In these embodiments, the channelrhodopsin fusion protein can include a channelrhodopsin linked to an inner mitochondrial membrane-mitochondrial localization signal (IMM-MLS), and the luciferase fusion protein can include a luciferase protein linked to an outer mitochondrial membrane-mitochondrial localization signal (OMM-MLS).

For example, the channelrhodopsin can be selected from Chloromonas oogama channelrhodopsin (CoChR), ChR2 (h134R), CHIEF, ChrimsonR, Chronos, CsChR, hChR2(C128A), hChR2(C128S), VChR1, and C1V1.

The IMM-MLS can be a leading sequence from a mitochondrial inner membrane protein selected from ABCB10, ABCB140, Cytochrome C, and renal outer medullary potassium channel (ROMK). The OMM-MLS can be OMA25 or TOM20 mitochondrial targeting sequence.

In some embodiments, the nucleic acid sequence encoding a channelrhodopsin fusion protein and the nucleic acid sequence encoding a luciferase fusion protein are operably linked to the same expression control sequence and are separated by a self-cleavable linker or internal ribosome entry site (IRES).

In some embodiments, the expression control sequence is a constitutive promoter. In some embodiments, the expression control sequence is a tissue specific promoter or cancer-specific promoter.

The system can in some embodiments further comprise a reporter gene to confirm transduction. For example, the reporter gene can encode a fluorophore. Example fluorophores include mCherry, eYFP, and dTomado.

The vector can be any vector suitable for transduction of cancer cells in a subject. In some embodiments, the vector comprises a viral vector. For example, the viral vector can be an adeno-associated virus (AAV) vector, adenovirus and lentivirus.

Also disclosed is a method for treating cancer in a subject that involves administering to the subject an expression vector containing nucleic acids encoding the disclosed fusion protein(s) operably linked to expression control sequences. The cancer can be any cell that can be targeted with a vector system, such as an AAV viral vector. In some embodiments, the cancer is a glioblastoma multiforme (GBM), a breast cancer, or a neuroendocrine tumor.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A to 1D show adenovirus-induced ChR2 expression in U251 (FIG. 1A), U-TMZ (FIG. 1B), PDX JX59 (FIG. 10) and PDX JX59T (FIG. 1D) mitochondria. ChR2 is fused with eYFP and mitochondria were stained with MitoTracker. Images were recorded using confocal microscope.

FIG. 2 shows blue light illumination induces mitochondrial depolarization in H9C2 myoblast cells expressing ABCB-ChR2 (fused with eYFP) (top) but not in those expressing ABCB-eYFP (bottom). Mitochondria were stained with TMRM for membrane potential recording.

FIG. 3 shows 12-hours blue light illumination induced Δψₘ depolarization in TMZ-resistant JX59T cells expressing ABCB-ChR2 (fused with eYFP). Cells were stained with Δψₘ dye mitoView 633 and Δψₘ were measured using flow cytometry.

FIG. 4 shows light illumination caused significant cytotoxicity in ABCB-ChR2, but not ABCB-eYFP expressing HeLa cells.

FIG. 5 shows evaluation of the expression and localization of CoChR in Cancer cell (NET) by confocal microscopy.

FIG. 6 shows NET Tumor in vitro treatment with CoChR and LED light.

FIG. 7 shows NET Tumor in vitro treatment with bioilluminance and no external light.

FIGS. 8A and 8B show NET animal study in vivo treatment. FIG. 8A shows adenovirus carrying mitochondrial-targeted chemo-optogenetic gene plus the RLuc substrate caused significant reduction in tumor growth in BON xenograft mice, while the adenovirus or substrate alone had no effect on tumor compared with control. FIG. 8B shows representative tumors images of the treated or untreated mice harvested at the end of study (10 days post treatment).

FIG. 9 shows breast cancer (BC) in vitro treatment. No external light is used.

FIG. 10 shows glioblastoma multiform (GBM) in vitro treatment.

FIG. 11 shows evaluation of cancer specific promoters by confocal microscopy.

FIG. 12 illustrates an IPD-pLenti-Vector.

FIG. 13 illustrates a IPD-pcDNA3.0-Plasmid.

FIG. 14 illustrates a pAD-CMV-Vector.

FIGS. 15A and 15B show in vivo evaluation of cancer specificity of targeted exosomes. FIG. 15A shows IVIS—cancer specific targeting and biodistribution of mAb, exosomes, and mAb-exosomes 24 hr post injection in NET xenograft mouse model. FIG. 15B shows IVIS—cancer specific targeting and biodistribution of mAb-exosomes in tumor and organs 24 hr post injection.

### DETAILED DESCRIPTION

Disclosed herein is a mitochondrial-targeting optogenetic approach to treat cancers, such as chemoresistant glioma and triple negative breast cancer, that involves targeted expression of rhodopsin proteins in mitochondria of cancer cells. One advantage of this approach is that mitochondrial optogenetics-mediated light-induced Δψₘ depolarization is independent of endogenous proteins (e.g. mitochondrial permeability transition pore, or mPTP). In addition, the mitochondrial expression of a channelrhodopsin can be targeted to the specific tumor tissue via transduction by a viral vector, such as adeno-associated virus (AAV), with appropriate combination of AAV serotype and promoter. Moreover, optogenetics is not simply photoexcitation of targeted cells; rather, optogenetics delivers gain of function of precise events. Thus optogenetic-mediated Δψₘ depolarization requires very low energy (usually at mW/mm² level), which would cause minimal effect on the proximal non-tumor tissue. Taken together, the mitochondrial-targeting optogenetics strategy, which induces apoptosis by directly disrupting IMM integrity, provides an attractive means of selectively eliminating cancerous cells and should be more effective against tumor acquired heterogeneity and drug resistance.

Optogenetics is an innovative technique that utilizes genetically encoded light-sensitive rhodopsin proteins, such as Chloromonas oogama Channelrhodopsin (CoChR), to remotely and precisely control the activity of cells. Since its discovery, optogenetics has been used to monitor and manipulate membrane excitability of a variety of types of cells such as neurons, cardiomyocytes, and stem cells (Zhang F, et al. Nature methods. 2006 3(10):785-92), as well as cancer cells (Yang F, et al. Cell Death Dis. 2013 4:e893; Kim K D, et al. Nat Commun. 2017 8:15365). Channelrhodopsins, such as CoChR, can be functionally expressed in mitochondria, allowing light-induced Δψₘ depolarization. As disclosed herein, mitochondrial optogenetics-mediated direct control of Δψₘ, an event that is independent of any endogenous protein, can overcome chemoresistance and induce cell death in heterogeneous glioma cells.

The disclosed subject matter can be understood more readily by reference to the following detailed description, the Figures, and the examples included herein.

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed. As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

It is understood that the disclosed methods and systems are not limited to the particular methodology, protocols, and systems described as these may vary. It is also to be understood that the terminology used herein is for the purpose of describing particular embodiments only, and is not intended to limit the scope of the present invention which will be limited only by the appended claims.

### Definitions

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

As used in the specification and the appended claims, the singular forms “a,” “an” and “the” include plural referents unless the context clearly dictates otherwise.

The word “or” as used herein means any one member of a particular list and can also include any combination of members of that list.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “optional” or “optionally” means that the subsequently described event or circumstance can or cannot occur, and that the description includes instances where said event or circumstance occurs and instances where it does not.

As used herein, the term “subject” refers to the target of administration, e.g., an animal. Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a patient. A patient refers to a subject afflicted with a disease or disorder, such as, for example, cancer and/or aberrant cell growth. The term “patient” includes human and veterinary subjects. In an aspect, the subject has been diagnosed with a need for treatment for cancer and/or aberrant cell growth.

The terms “treating”, “treatment”, “therapy”, and “therapeutic treatment” as used herein refer to curative therapy, prophylactic therapy, or preventative therapy. As used herein, the terms refers to the medical management of a subject or a patient with the intent to cure, ameliorate, stabilize, or prevent a disease, pathological condition, or disorder, such as, for example, cancer or a tumor. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In an aspect, the disease, pathological condition, or disorder is cancer, such as, for example, breast cancer, lung cancer, colorectal, liver cancer, or pancreatic cancer. In an aspect, cancer can be any cancer known to the art.

As used herein, the terms “administering” and “administration” refer to any method of providing a composition to a subject. Such methods are well known to those skilled in the art and include, but are not limited to, intracardiac administration, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed composition or peptide or pharmaceutical preparation and a cell, target receptor, or other biological entity together in such a manner that the compound can affect the activity of the target (e.g., receptor, transcription factor, cell, etc.), either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, in an aspect, an effective amount of the polymeric nanoparticle is an amount that kills and/or inhibits the growth of cells without causing extraneous damage to surrounding non-cancerous cells. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts.

The term “pharmaceutically acceptable” describes a material that is not biologically or otherwise undesirable, i.e., without causing an unacceptable level of undesirable biological effects or interacting in a deleterious manner. The term “pharmaceutically acceptable carrier” includes sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate.

As used herein, the term “cancer” refers to a proliferative disorder or disease caused or characterized by the proliferation of cells which have lost susceptibility to normal growth control. The term “cancer” includes tumors and any other proliferative disorders. Cancers of the same tissue type originate in the same tissue, and can be divided into different subtypes based on their biological characteristics. Cancer includes, but is not limited to, melanoma, leukemia, astrocytoma, glioblastoma, lymphoma, glioma, Hodgkin's lymphoma, and chronic lymphocyte leukemia. Cancer also includes, but is not limited to, cancer of the brain, bone, pancreas, lung, liver, breast, thyroid, ovary, uterus, testis, pituitary, kidney, stomach, esophagus, anus, and rectum.

“Promoter” as used herein means a synthetic or naturally-derived molecule which is capable of conferring, activating or enhancing expression of a polynucleotide in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant vectors express genes that are not found within the native (naturally occurring) form of the vector or express a second copy of a native gene that is otherwise normally or abnormally expressed, under expressed or not expressed at all.

The term “gene therapy” as used herein means genetic modification of cells by the introduction of exogenous DNA or RNA into these cells for the purpose of expressing or replicating one or more peptides, polypeptides, proteins, oligonucleotides, or polynucleotides in vivo for the treatment or prevention of disease or deficiencies in humans or animals. Gene therapy is generally disclosed in U.S. Pat. No. 5,399,346. Any suitable route or routes of administration of the nucleic acid or protein may be employed for providing a subject with pharmaceutical compositions of the presently disclosed inventive constructs, optionally in combination with one or more pharmaceutical agents. For example, parenteral (subcutaneous, subretinal, suprachoroidal, intramuscular, intravenous, transdermal, intracranial) and like forms of administration may be employed alone or in combination. Dosage formulations include injections, implants, or other known and effective gene therapy delivery methods.

As used herein, the term “Adeno-associated virus” or “AAV” as used interchangeably herein refers to a small virus belonging to the genus Dependovirus of the Parvoviridae family that infects humans and some other primate species. “AAV” encompasses natural and engineered AAV 1, 2, 3, 4, 5, 6, 7, 8, 9, and rh10 variants. In some embodiments, variants have about 60% sequence identity, and in some embodiments 65%, 70%, 75%, 80%, 85%, 90%, 91%, 92%, 93%, 94%, 95%, 96%, 97%, 98%, 99%, or higher identity over a specified region. AAV is not currently known to cause disease and consequently the virus causes a very mild immune response. A number of AAV serotypes are known and well-described in the art. Embodiments described herein may employ any of AAV serotypes 1, 2, 3, 4, 5, 6, 7, 8, 9, and rh10. Nucleotide sequences of these serotypes are readily available, and, for the sake of brevity, are not provided herein.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

Mitochondrial Optogenetics-Based Gene Therapy System

Fusion Proteins

The disclosed optogenetics-based gene therapy involves channelrhodopsin fusion proteins, nucleic acids encoding the fusion proteins, and vector systems for expressing these nucleic acids and proteins in cancer cells. The channelrhodopsin fusion protein has at least two domains: 1) an inner mitochondrial membrane-mitochondrial localization signal (IMM-MLS) that can effectively target the fusion protein to an inner mitochondria membrane, and 2) a channelrhodopsin ion channel domain that can change the mitochondrial membrane potential (Δψₘ) when light is present.

The disclosed optogenetics-based gene therapy system can in some embodiments further involve luciferase fusion proteins to stimulate the channelrhodopsin without reliance on external light, as well as nucleic acids encoding the fusion proteins, and vector systems for co-expressing these nucleic acids and proteins in cancer cells with the channelrhodopsin fusion proteins. The luciferase fusion protein has at least two domains: 1) an outer mitochondrial membrane-mitochondrial localization signal (OMM-MLS) that can effectively target the luciferase fusion protein to an outer mitochondrial membrane, and 2) a luciferase protein that can produce a bioluminescence in the presence of a luciferase substrate.

Fusion proteins, also known as chimeric proteins, are proteins created through the joining of two or more genes which originally coded for separate proteins. Translation of this fusion gene results in a single polypeptide with function properties derived from each of the original proteins. Recombinant fusion proteins can be created artificially by recombinant DNA technology for use in biological research or therapeutics. Chimeric mutant proteins occur naturally when a large-scale mutation, typically a chromosomal translocation, creates a novel coding sequence containing parts of the coding sequences from two different genes.

The functionality of fusion proteins is made possible by the fact that many protein functional domains are modular. In other words, the linear portion of a polypeptide which corresponds to a given domain, such as a tyrosine kinase domain, may be removed from the rest of the protein without destroying its intrinsic enzymatic capability.

A recombinant fusion protein is a protein created through genetic engineering of a fusion gene. This typically involves removing the stop codon from a cDNA sequence coding for the first protein, then appending the cDNA sequence of the second protein in frame through ligation or overlap extension PCR. That DNA sequence will then be expressed by a cell as a single protein.

Gene Expression Systems

Also disclosed are nucleic acids encoding the disclosed fusion proteins operably linked to expression control sequences, such as promoters and enhancers.

In some embodiments, the promoter is a constitutive promoter. For example, the cytomegalovirus (CMV) early enhancer/promoter and the hybrid CMV enhancer/chicken β-actin (CBA) promoter are commonly used in gene transfer studies with therapeutic genes. In particular, the “CAG promoter” is a strong synthetic promoter constructed from the CMV early enhancer element; the promoter, the first exon and the first intron of chicken β-actin gene; and the splice acceptor of the rabbit β-globin gene. These promoters are typically used to provide robust, long-term expression in all cell types. The 800-bp CMV enhancer/promoter has often been used to achieve rapid and ubiquitous expression.

In some embodiments, the promoter is tissue specific. For example, the human synapsin I (SynI) gene promoter confers highly neuron-specific long-term transgene expression from an adenoviral vector, which transduces mainly glial cells in the brain. Likewise, the Ca²⁺/calmodulin-dependent protein kinase II (CaMKII) promoter is neuron specific for excitatory neurons. The chromogranin A (CgA) promoter can be used for cell-specific gene expression in the gastroenteropancreatic neuroendocrine system.

In some embodiments, the promoter is tumor specific. For example, the insulinoma-associated 1 (INSM1) gene is expressed exclusively during early embryonal development, but has been found re-expressed at high levels in neuroendocrine tumors. The regulatory region of the INSM1 gene is therefore a potential candidate for regulating expression of a therapeutic gene in transcriptionally targeted cancer gene therapy against neuroendocrine tumors.

The nucleic acids encoding the channelrhodopsin fusion protein and the luciferase fusion protein can be present on the same polynucleotide. In these embodiments, the nucleic acids encoding the channelrhodopsin fusion protein and the luciferase fusion protein can be operably linked to separate promoters or the same promoter. In the embodiments where the fusion proteins share a promoter, they can be separated, for example, by a linker.

Linker (or “spacer”) peptides can be added to make it more likely that the two fusion proteins fold independently and behave as expected. Linkers in protein or peptide fusions are sometimes engineered with cleavage sites for proteases or chemical agents which enable the liberation of the two separate proteins. Alternatively, internal ribosome entry sites (IRES) elements can be used to create multigene, or polycistronic, messages. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

In some cases, the linker is a self-cleaving peptide. For example, the 2A self-cleaving peptide (2A), which was discovered in the foot-and-mouth-disease virus (FMDV), is an oligopeptide (usually 19-22 amino acids) located between two proteins in some members of the picornavirus family. 2A-like sequences in other viral mRNA molecules have been successfully identified, including the porcine teschovirus-1 2A (P2A), thosea asigna virus 2A (T2A), equine rhinitis A virus 2A (E2A), cytoplasmic polyhedrosis virus (BmCPV 2A), and flacherie virus (BmIFV 2A) of B. mori.

The system can in some embodiments further comprise a reporter gene to confirm transduction. For example, the reporter gene can encode a fluorophore. Example fluorophores include mCherry, yYFP, and dTomado.

Viral Vectors

The disclosed gene therapy system may comprise a vector comprising a nucleotide sequence encoding the disclosed fusion protein(s). The vector may further comprise initiation and termination signals operably linked to regulatory elements including a promoter. The vector may further include a polyadenylation signal capable of directing expression in the cells of an individual or mammal to which the gene therapy system is administered. The vector may further comprise a reporter gene, such as GFP. The vector may further comprise a selectable marker.

The vector delivery system may be any vector delivery system. The vector may be a viral vector. Any viral vector or hybrid thereof may be used.

The viral vector may be an adenoviral vector. The vector may be an adeno-associated virus (AAV) vector. The AAV vector is a small virus belonging to the genus Dependovirus of the Parvoviridae family that infects humans and other primate species. AAV may be an attractive vector system for use according to the present invention as it has a high frequency of integration and it can infect non-dividing cells, thus making it useful for delivery of genes into mammalian cells, for example, in tissue culture or in vivo. AAV has a broad host range for infectivity.

The AAV vector may be a modified AAV vector. The modified AAV vector may have enhanced tissue tropism. The modified AAV vector may be capable of delivering and expressing the disclosed fusion protein(s) in the cell of a mammal. The modified AAV vector may be based on one or more of several capsid types, including AAV1, AAV2, AAV5, AAV6, AAV8, and AAV9.

The vector may be a retroviral vector. Retroviruses have promise as gene delivery vectors due to their ability to integrate their genes into the host genome, transferring a large amount of foreign genetic material, infecting a broad spectrum of species and cell types and of being packaged in special cell-lines.

The vector may be a lentiviral vector. Lentiviruses are complex retroviruses, which, in addition to the common retroviral genes gag, pol, and env, contain other genes with regulatory or structural function. Some examples of lentivirus include the Human Immunodeficiency Viruses (HIV-1, HIV-2) and the Simian Immunodeficiency Virus (SIV). Lentiviral vectors have been generated by multiply attenuating the HIV virulence genes, for example, the genes env, vif, vpr, vpu and nef are deleted making the vector biologically safe.

Other viral vectors may be employed as constructs in the disclosed gene therapy system. Vectors derived from viruses such as vaccinia virus, Epstein-Barr virus, sindbis virus, cytomegalovirus and herpes simplex virus may be employed.

In embodiments, the viral vector includes any vector that can effectively transduce cancer cells and express the disclosed fusion protein(s). In embodiments, the viral vector is a lentiviral vector. In other embodiments, the viral vector is an adeno-associated virus (AAV) vector.

The AAV serotype 9 (AAV9) has been shown to cross the blood brain barrier and preferentially transduce neurons in neonates and astrocytes in adults. AAV9 effectively eliminates the need for direct injection into the CNS as systemic delivery should reach CNS targets.

Sequences

Amino acid and nucleic acid sequences for the disclosed fusion protein domains are generally known in the art. Example proteins are provided below, but others known in the art are not provided for the sake of brevity.

Channelrhodopsin

Suitable channelrhodopsins include light sensitive ion channel protein CoChR, ChR2(h134R), CHIEF, ChrimsonR, Chronos, CsChR, hChR2(C128A), hChR2(C128S), VChR1, and C1V1. In particular embodiments, the channelrhodopsin is the light sensitive ion channel protein CoChR, which can have the amino acid sequence:

(SEQ ID NO: 1)
MLGNGSAIVPIDQCFCLAWTDSLGSDTEQLVANILQWFAFGFSILILMF
YAYQTWRATCGWEEVYVCCVELTKVIIEFFHEFDDPSMLYLANGHRVQW
LRYAEWLLTCPVILIHLSNLTGLKDDYSKRTMRLLVSDVGTIVWGATSA
MSTGYVKVIFFVLGCIYGANTFFHAAKVYIESYHVVPKGRPRTVVRIMA
WLFFLSWGMFPVLFVVGPEGFDAISVYGSTIGHTIIDLMSKNCWGLLGH
YLRVLIHQHIIIYGDIRKKTKINVAGEEMEVETMVDQEDEETV.

CoChR can be encoded by the nucleic acid sequence:

(SEQ ID NO: 2)
ATGCTGGGAAACGGCAGCGCCATTGTGCCTATCGACCAGTGCTTTTGCC
TGGCTTGGACCGACAGCCTGGGAAGCGATACAGAGCAGCTGGTGGCCAA
CATCCTCCAGTGGTTCGCCTTCGGCTTCAGCATCCTGATCCTGATGTTC
TACGCCTACCAGACTTGGAGAGCCACTTGCGGTTGGGAGGAGGTCTACG
TCTGTTGCGTCGAGCTGACCAAGGTCATCATCGAGTTCTTCCACGAGTT
CGACGACCCCAGCATGCTGTACCTGGCTAACGGACACCGAGTCCAGTGG
CTGAGATACGCAGAGTGGCTGCTGACTTGTCCCGTCATCCTGATCCACC
TGAGCAACCTGACCGGCCTGAAGGACGACTACAGCAAGCGGACCATGAG
GCTGCTGGTGTCAGACGTGGGAACCATCGTGTGGGGAGCTACAAGCGCC
ATGAGCACAGGCTACGTCAAGGTCATCTTCTTCGTGCTGGGTTGCATCT
ACGGCGCCAACACCTTCTTCCACGCCGCCAAGGTGTATATCGAGAGCTA
CCACGTGGTGCCAAAGGGCAGACCTAGAACCGTCGTGCGGATCATGGCT
TGGCTGTTCTTCCTGTCTTGGGGCATGTTCCCCGTGCTGTTCGTCGTGG
GACCAGAAGGATTCGACGCCATCAGCGTGTACGGCTCTACCATTGGCCA
CACCATCATCGACCTCATGAGCAAGAATTGTTGGGGCCTGCTGGGACAC
TATCTGAGAGTGCTGATCCACCAGCACATCATCATCTACGGCGACATCC
GGAAGAAGACCAAGATCAACGTGGCCGGCGAGGAGATGGAAGTGGAGAC
CATGGTGGACCAGGAGGACGAGGAGACAGTG.

In some embodiments, the channelrhodopsin is ChR2(h134R), which can have the amino acid sequence:

(SEQ ID NO: 3)
MDYGGALSAVGRELLFVTNPVVVNGSVLVPEDQCYCAGWIESRGTNGAQ
TASNVLQWLAAGFSILLLMFYAYQTWKSTCGWEEIYVCAIEMVKVILEF
FFEFKNPSMLYLATGHRVQWLRYAEWLLTCPVILIRLSNLTGLSNDYSR
RTMGLLVSDIGTIVWGATSAMATGYVKVIFFCLGLCYGANTFFHAAKAY
IEGYHTVPKGRCRQVVTGMAWLFFVSWGMFPILFILGPEGFGVLSVYGS
TVGHTIIDLMSKNCWGLLGHYLRVLIHEHILIHGDIRKTTKLNIGGTEI
EVETLVEDEAEAGAVPAAATMVSKGEELFTGVVPILVELDGDVNGHKFS
VSGEGEGDATYGKLTLKFICTTGKLPVPWPTLVTTFGYGLQCFARYPDH
MKQHDFFKSAMPEGYVQERTIFFKDDGNYKTRAEVKFEGDTLVNRIELK
GIDFKEDGNILGHKLEYNYNSHNVYIMADKQKNGIKVNFKIRHNIEDGS
VQLADHYQQNTPIGDGPVLLPDNHYLSYQSALSKDPNEKRDHMVLLEFV
TAAGITLGMDELYK.

That can be encoded by the nucleic acid sequence:

(SEQ ID NO: 4)
ATGGACTATGGCGGCGCTTTGTCTGCCGTCGGACGCGAACTTTTGTTCG
TTACTAATCCTGTGGTGGTGAACGGGTCCGTCCTGGTCCCTGAGGATCA
ATGTTACTGTGCCGGATGGATTGAATCTCGCGGCACGAACGGCGCTCAG
ACCGCGTCAAATGTCCTGCAGTGGCTTGCAGCAGGATTCAGCATTTTGC
TGCTGATGTTCTATGCCTACCAAACCTGGAAATCTACATGCGGCTGGGA
GGAGATCTATGTGTGCGCCATTGAAATGGTTAAGGTGATTCTCGAGTTC
TTTTTTGAGTTTAAGAATCCCTCTATGCTCTACCTTGCCACAGGACACC
GGGTGCAGTGGCTGCGCTATGCAGAGTGGCTGCTCACTTGTCCTGTCAT
CCTTATCCGCCTGAGCAACCTCACCGGCCTGAGCAACGACTACAGCAGG
AGAACCATGGGACTCCTTGTCTCAGACATCGGGACTATCGTGTGGGGGG
CTACCAGCGCCATGGCAACCGGCTATGTTAAAGTCATCTTCTTTTGTCT
TGGATTGTGCTATGGCGCGAACACATTTTTTCACGCCGCCAAAGCATAT
ATCGAGGGTTATCATACTGTGCCAAAGGGTCGGTGCCGCCAGGTCGTGA
CCGGCATGGCATGGCTGTTTTTCGTGAGCTGGGGTATGTTCCCAATTCT
CTTCATTTTGGGGCCCGAAGGTTTTGGCGTCCTGAGCGTCTATGGCTCC
ACCGTAGGTCACACGATTATTGATCTGATGAGTAAAAATTGTTGGGGGT
TGTTGGGACACTACCTGCGCGTCCTGATCCACGAGCACATATTGATTCA
CGGAGATATCCGCAAAACCACCAAACTGAACATCGGCGGAACGGAGATC
GAGGTCGAGACTCTCGTCGAAGACGAAGCCGAGGCCGGAGCCGTGCCAG
CGGCCGCCACCATGGTGAGCAAGGGCGAGGAGCTGTTCACCGGGGTGGT
GCCCATCCTGGTCGAGCTGGACGGCGACGTAAACGGCCACAAGTTCAGC
GTGTCCGGCGAGGGCGAGGGCGATGCCACCTACGGCAAGCTGACCCTGA
AGTTCATCTGCACCACCGGCAAGCTGCCCGTGCCCTGGCCCACCCTCGT
GACCACCTTCGGCTACGGCCTGCAGTGCTTCGCCCGCTACCCCGACCAC
ATGAAGCAGCACGACTTCTTCAAGTCCGCCATGCCCGAAGGCTACGTCC
AGGAGCGCACCATCTTCTTCAAGGACGACGGCAACTACAAGACCCGCGC
CGAGGTGAAGTTCGAGGGCGACACCCTGGTGAACCGCATCGAGCTGAAG
GGCATCGACTTCAAGGAGGACGGCAACATCCTGGGGCACAAGCTGGAGT
ACAACTACAACAGCCACAACGTCTATATCATGGCCGACAAGCAGAAGAA
CGGCATCAAGGTGAACTTCAAGATCCGCCACAACATCGAGGACGGCAGC
GTGCAGCTCGCCGACCACTACCAGCAGAACACCCCCATCGGCGACGGCC
CCGTGCTGCTGCCCGACAACCACTACCTGAGCTACCAGTCCGCCCTGAG
CAAAGACCCCAACGAGAAGCGCGATCACATGGTCCTGCTGGAGTTCGTG
ACCGCCGCCGGGATCACTCTCGGCATGGACGAGCTGTACAAGTAA.

In some embodiments, the channelrhodopsin is CHIEF, which can have the amino acid sequence

(SEQ ID NO: 5)
MTMVSRRPWLLALALAVALAAGSAGASTGSDATVPVATQDGPDYVFHRA
HERMLFQTSYTLENNGSVICIPNNGQCFCLAWLKSNGTNAEKLAANILQ
WITFALSALCLMFYGYQTWKSTCGWEEIYVATIEMIKFIIEYFHEFDEP
AVIYSSNGNKTVWLRYAEWLLTCPVVLIHLSNLTGLANDYNKRTMGLLV
SDIGTIVWGTTAALSKGYVRVIFFLMGLCYGIYTFFNAAKVYIEAYHTV
PKGRCRQVVTGMAWLFFVSWGMFPILFILGPEGFGVLSVYGSTVGHTII
DLMSKNCWGLLGHYLRVLIHEHILIHGDIRKTTKLNIGGTEIEVETLVE
DEAEAGAVNKGTGKYESS.

That can be encoded by the nucleic acid sequence:

(SEQ ID NO: 6)
ACCATGGTGAGCAGAAGACCCTGGCTGCTGGCCCTGGCCCTGGCCGTGG
CCCTGGCCGCCGGCAGCGCCGGCGCCAGCACCGGCAGCGACGCCACCGT
GCCCGTGGCCACCCAGGACGGCCCCGACTACGTGTTCCACAGAGCCCAC
GAGAGAATGCTGTTCCAGACCAGCTACACCCTGGAGAACAACGGCAGCG
TGATCTGCATCCCCAACAACGGCCAGTGCTTCTGCCTGGCCTGGCTGAA
GAGTAACGGCACCAACGCCGAGAAGCTGGCCGCCAACATCCTGCAGTGG
ATCACCTTCGCCCTGAGCGCCCTGTGCCTGATGTTCTACGGCTACCAGA
CCTGGAAGAGTACCTGCGGCTGGGAGGAGATCTACGTGGCCACCATCGA
GATGATCAAGTTCATCATAGAGTACTTCCACGAGTTCGACGAGCCCGCC
GTGATCTACAGCAGCAACGGCAACAAGACCGTGTGGCTGAGATACGCCG
AGTGGCTGCTGACCTGCCCCGTGGTCCTGATCCACCTGAGCAACCTGAC
CGGCCTGGCCAACGACTACAACAAGAGAACCATGGGCCTGCTGGTGAGC
GACATCGGCACCATCGTGTGGGGCACCACCGCCGCCCTGAGCAAGGGCT
ACGTGAGAGTGATCTTCTTCCTGATGGGCCTGTGCTACGGCATCTACAC
CTTCTTCAACGCCGCCAAGGTGTACATCGAGGCCTACCACACCGTGCCC
AAGGGCAGATGCAGACAGGTGGTGACCGGCATGGCCTGGCTGTTCTTCG
TGAGCTGGGGCATGTTCCCCATCCTGTTCATCCTGGGCCCCGAGGGCTT
CGGCGTGCTGAGCGTGTACGGCAGCACCGTGGGCCACACCATCATCGAC
CTGATGAGCAAGAACTGCTGGGGCCTGCTGGGCCACTACCTGAGAGTGC
TGATCCACGAGCACATCCTGATCCACGGCGACATCAGAAAGACCACCAA
GCTGAACATCGGCGGCACCGAGATCGAGGTGGAGACCCTGGTGGAGGAC
GAGGCCGAGGCCGGCGCCGTGAACAAGGGCACCGGCAAGTACGAGAGCA
GC.

In some embodiments, the channelrhodopsin is ChrimsonR, which can have the amino acid sequence:

(SEQ ID NO: 7)
MAELISSATRSLFAAGGINPWPNPYHHEDMGCGGMTPTGECFSTEWWCDPS
YGLSDAGYGYCFVEATGGYLVVGVEKKQAWLHSRGTPGEKIGAQVCQWIAF
SIAIALLTFYGFSAWKATCGWEEVYVCCVEVLFVTLEIFKEFSSPATVYLS
TGNHAYCLRYFEWLLSCPVILIRLSNLSGLKNDYSKRTMGLIVSCVGMIVF
GMAAGLATDWLKWLLYIVSCIYGGYMYFQAAKCYVEANHSVPKGHCRMVVK
LMAYAYFASWGSYPILWAVGPEGLLKLSPYANSIGHSICDIIAKEFWTFLA
HHLRIKIHEHILIHGDIRKTTKMEIGGEEVEVEEFVEEEDEDTVAAPVVAV
SKGEEVIKEFMRFKVRMEGSMNGHEFEIEGEGEGRPYEGTQTAKLKVTKGG
PLPFAWDILSPQFMYGSKAYVKHPADIPDYKKLSFPEGFKWERV.

That can be encoded by the nucleic acid sequence:

(SEQ ID NO: 8)
ATGGCTGAGCTGATCAGCAGCGCCACCAGATCTCTGTTTGCCGCCGGAGGC
ATCAACCCTTGGCCTAACCCCTACCACCACGAGGACATGGGCTGTGGAGGA
ATGACACCTACAGGCGAGTGCTTCAGCACCGAGTGGTGGTGTGACCCTTCT
TACGGACTGAGCGACGCCGGATACGGATATTGCTTCGTGGAGGCCACAGGC
GGCTACCTGGTCGTGGGAGTGGAGAAGAAGCAGGCTTGGCTGCACAGCAGA
GGCACACCAGGAGAAAAGATCGGCGCCCAGGTCTGCCAGTGGATTGCTTTC
AGCATCGCCATCGCCCTGCTGACATTCTACGGCTTCAGCGCCTGGAAGGCC
ACTTGCGGTTGGGAGGAGGTCTACGTCTGTTGCGTCGAGGTGCTGTTCGTG
ACCCTGGAGATCTTCAAGGAGTTCAGCAGCCCCGCCACAGTGTACCTGTCT
ACCGGCAACCACGCCTATTGCCTGCGCTACTTCGAGTGGCTGCTGTCTTGC
CCCGTGATCCTGATCAGACTGAGCAACCTGAGCGGCCTGAAGAACGACTAC
AGCAAGCGGACCATGGGCCTGATCGTGTCTTGCGTGGGAATGATCGTGTTC
GGCATGGCCGCAGGACTGGCTACCGATTGGCTCAAGTGGCTGCTGTATATC
GTGTCTTGCATCTACGGCGGCTACATGTACTTCCAGGCCGCCAAGTGCTAC
GTGGAAGCCAACCACAGCGTGCCTAAAGGCCATTGCCGCATGGTCGTGAAG
CTGATGGCCTACGCTTACTTCGCCTCTTGGGGCAGCTACCCAATCCTCTGG
GCAGTGGGACCAGAAGGACTGCTGAAGCTGAGCCCTTACGCCAACAGCATC
GGCCACAGCATCTGCGACATCATCGCCAAGGAGTTTTGGACCTTCCTGGCC
CACCACCTGAGGATCAAGATCCACGAGCACATCCTGATCCACGGCGACATC
CGGAAGACCACCAAGATGGAGATCGGAGGCGAGGAGGTGGAAGTGGAAGAG
TTCGTGGAGGAGGAGGACGAGGACACAGTGGCGGCACCGGTAGTAGCAGTG
AGTAAGGGCGAGGAAGTGATCAAAGAGTTCATGCGGTTTAAGGTGAGAATG
GAAGGAAGCATGAACGGCCACGAGTTCGAAATTGAGGGAGAAGGAGAGGGA
CGGCCCTACGAGGGCACCCAGACAGCCAAGCTGAAAGTGACAAAGGGCGGG
CCTCTGCCATTCGCTTGGGACATCCTGAGCCCACAGTTTATGTACGGCTCC
AAGGCCTATGTGAAACATCCAGCTGACATTCCCGATTATAAGAAACTGAGC
TTCCCCGAGGGGTTTAAGTGGGAAAGAGTG.

In some embodiments, the channelrhodopsin is Chronos, which can have the following amino acid sequence

(SEQ ID NO: 9)
MTHAFISAVPSAEATIRGLLSAAAVVTPAADAHGETSNATTAGADHGCFPH
INHGTELQHKIAVGLQWFTVIVAIVQLIFYGWHSFKATTGWEEVYVCVIEL
VKCFIELFHEVDSPATVYQTNGGAVIWLRYSMWLLTCPVILIHLSNLTGLH
EEYSKRTMTILVTDIGNIVWGITAAFTKGPLKILFFMIGLFYGVTCFFQIA
KVYIESYHTLPKGVCRKICKIMAYVFFCSWLMFPVMFIAGHEGLGLITPYT
SGIGHLILDLISKNTWGFLGHHLRVKIHEHILIHGDIRKTTTINVAGENME
IETFVDEEEEGGVAAPVVA.

In some embodiments, the channelrhodopsin is encoded by the nucleic acid sequence:

(SEQ ID NO: 10)
ATGACCCACGCCTTTATCTCAGCCGTGCCTAGCGCCGAAGCCACAATTAGA
GGCCTGCTGAGCGCCGCAGCAGTGGTGACACCAGCAGCAGACGCTCACGGA
GAAACCTCTAACGCCACAACAGCCGGAGCCGATCACGGTTGCTTCCCCCAC
ATCAACCACGGAACCGAGCTGCAGCACAAGATCGCAGTGGGACTCCAGTGG
TTCACCGTGATCGTGGCTATCGTGCAGCTCATCTTCTACGGTTGGCACAGC
TTCAAGGCCACAACCGGCTGGGAGGAGGTCTACGTCTGCGTGATCGAGCTC
GTCAAGTGCTTCATCGAGCTGTTCCACGAGGTCGACAGCCCAGCCACAGTG
TACCAGACCAACGGAGGAGCCGTGATTTGGCTGCGGTACAGCATGTGGCTC
CTGACTTGCCCCGTGATCCTGATCCACCTGAGCAACCTGACCGGACTGCAC
GAAGAGTACAGCAAGCGGACCATGACCATCCTGGTGACCGACATCGGCAAC
ATCGTGTGGGGGATCACAGCCGCCTTTACAAAGGGCCCCCTGAAGATCCTG
TTCTTCATGATCGGCCTGTTCTACGGCGTGACTTGCTTCTTCCAGATCGCC
AAGGTGTATATCGAGAGCTACCACACCCTGCCCAAAGGCGTCTGCCGGAAG
ATTTGCAAGATCATGGCCTACGTCTTCTTCTGCTCTTGGCTGATGTTCCCC
GTGATGTTCATCGCCGGACACGAGGGACTGGGCCTGATCACACCTTACACC
AGCGGAATCGGCCACCTGATCCTGGATCTGATCAGCAAGAACACTTGGGGC
TTCCTGGGCCACCACCTGAGAGTGAAGATCCACGAGCACATCCTGATCCAC
GGCGACATCCGGAAGACAACCACCATCAACGTGGCCGGCGAGAACATGGAG
ATCGAGACCTTCGTCGACGAGGAGGAGGAGGGAGGAGTGGCGGCACCGGTA
GTAGCA.

In some embodiments, the channelrhodopsin is CsChR, which can have the following amino acid sequence:

(SEQ ID NO: 11)
MSRLVAASWLLALLLCGITSTTTASSAPAASSTDGTAAAAVSHYAMNGFDE
LAKGAVVPEDHFVCGPADKCYCSAWLHSHGSKEEKTAFTVMQWIVFAVCII
SLLFYAYQTWRATCGWEEVYVTIIELVHVCFGLWHEVDSPCTLYLSTGNMV
LWLRYAEWLLTCPVILIHLSNLTGMKNDYNKRTMALLVSDVGCIVWGTTAA
LSTDFVKIIFFFLGLLYGFYTFYAAAKIYIEAYHTVPKGICRQLVRLQAYD
FFFTWSMFPILFMVGPEGFGKITAYSSGIAHEVCDLLSKNLWGLMGHFIRV
KIHEHILVHGNITKKTKVNVAGDMVELDTYVDQDEEHDEGAAPVVA.

In some embodiments, the channelrhodopsin is encoded by the nucleic acid sequence:

(SEQ ID NO: 12)
ATGAGCAGACTGGTCGCCGCTTCTTGGCTGCTGGCTCTCCTCCTCTGCGGA
ATTACCAGCACAACAACAGCCTCTAGCGCCCCAGCAGCTTCTTCTACAGAC
GGAACAGCCGCCGCAGCAGTGTCTCACTACGCCATGAACGGCTTCGACGAG
CTGGCTAAAGGAGCCGTGGTGCCAGAAGACCACTTTGTCTGCGGACCAGCC
GACAAGTGCTATTGCTCCGCTTGGCTGCACAGCCACGGAAGCAAGGAGGAG
AAGACCGCCTTCACCGTCATGCAGTGGATCGTGTTCGCCGTCTGCATCATC
AGCCTGCTGTTCTACGCCTACCAGACTTGGAGGGCTACTTGCGGTTGGGAG
GAGGTGTACGTGACCATCATCGAGCTGGTCCACGTCTGCTTCGGACTCTGG
CACGAGGTCGATAGCCCTTGTACCCTGTACCTGAGCACAGGCAACATGGTC
CTCTGGCTGAGATACGCCGAGTGGCTGCTGACTTGCCCCGTGATCCTGATC
CACCTGAGCAACCTGACCGGCATGAAGAACGACTACAACAAGCGGACCATG
GCCCTGCTGGTGTCAGACGTGGGCTGTATCGTGTGGGGAACAACAGCCGCC
CTGAGCACCGATTTCGTGAAGATCATCTTCTTCTTCCTGGGCCTGCTGTAC
GGCTTCTACACCTTCTACGCCGCCGCCAAGATCTACATCGAGGCCTACCAC
ACCGTGCCCAAGGGCATTTGTAGACAGCTCGTGCGGCTGCAGGCCTACGAC
TTCTTCTTCACTTGGAGCATGTTCCCCATCCTGTTCATGGTCGGCCCAGAG
GGATTCGGCAAGATCACCGCCTACAGCAGCGGAATCGCCCACGAAGTGTGC
GATCTGCTGAGCAAGAACCTCTGGGGCCTGATGGGCCACTTCATCCGCGTG
AAGATCCACGAGCACATCCTGGTGCACGGCAACATCACCAAGAAGACCAAG
GTCAACGTGGCCGGCGACATGGTGGAACTGGACACCTACGTGGACCAGGAC
GAGGAACACGACGAGGGAGCGGCACCGGTAGTAGCA.

In some embodiments, the channelrhodopsin is hChR2(C128A), which can have the following amino acid sequence

(SEQ ID NO: 13)
MDYGGALSAVGRELLFVTNPVVVNGSVLVPEDQCYCAGWIESRGTNGAQTA
SNVLQWLAAGFSILLLMFYAYQTWKSTCGWEEIYVCAIEMVKVILEFFFEF
KNPSMLYLATGHRVQWLRYAEWLLTAPVILIHLSNLTGLSNDYSRRTMGLL
VSDIGTIVWGATSAMATGYVKVIFFCLGLCYGANTFFHAAKAYIEGYHTVP
KGRCRQVVTGMAWLFFVSWGMFPILFILGPEGFGVLSVYGSTVGHTIIDLM
SKNCWGLLGHYLRVLIHEHILIHGDIRKTTKLNIGGTEIEVETLVEDEAEA
GAVP.

In some embodiments, the channelrhodopsin is encoded by the nucleic acid sequence:

(SEQ ID NO: 14)
ATGGACTATGGCGGCGCTTTGTCTGCCGTCGGACGCGAACTTTTGTTCGTT
ACTAATCCTGTGGTGGTGAACGGGTCCGTCCTGGTCCCTGAGGATCAATGT
TACTGTGCCGGATGGATTGAATCTCGCGGCACGAACGGCGCTCAGACCGCG
TCAAATGTCCTGCAGTGGCTTGCAGCAGGATTCAGCATTTTGCTGCTGATG
TTCTATGCCTACCAAACCTGGAAATCTACATGCGGCTGGGAGGAGATCTAT
GTGTGCGCCATTGAAATGGTTAAGGTGATTCTCGAGTTCTTTTTTGAGTTT
AAGAATCCCTCTATGCTCTACCTTGCCACAGGACACCGGGTGCAGTGGCTG
CGCTATGCAGAGTGGCTGCTCACTGCCCCTGTCATCCTTATCCACCTGAGC
AACCTCACCGGCCTGAGCAACGACTACAGCAGGAGAACCATGGGACTCCTT
GTCTCAGACATCGGGACTATCGTGTGGGGGGCTACCAGCGCCATGGCAACC
GGCTATGTTAAAGTCATCTTCTTTTGTCTTGGATTGTGCTATGGCGCGAAC
ACATTTTTTCACGCCGCCAAAGCATATATCGAGGGTTATCATACTGTGCCA
AAGGGTCGGTGCCGCCAGGTCGTGACCGGCATGGCATGGCTGTTTTTCGTG
AGCTGGGGTATGTTCCCAATTCTCTTCATTTTGGGGCCCGAAGGTTTTGGC
GTCCTGAGCGTCTATGGCTCCACCGTAGGTCACACGATTATTGATCTGATG
AGTAAAAATTGTTGGGGGTTGTTGGGACACTACCTGCGCGTCCTGATCCAC
GAGCACATATTGATTCACGGAGATATCCGCAAAACCACCAAACTGAACATC
GGCGGAACGGAGATCGAGGTCGAGACTCTCGTCGAAGACGAAGCCGAGGCC
GGAGCCGTGCCA.

In some embodiments, the channelrhodopsin is hChR2(C128S), which can have the flowing amino acid sequence:

(SEQ ID NO: 15)
MDYGGALSAVGRELLFVTNPVVVNGSVLVPEDQCYCAGWIESRGTNGAQTA
SNVLQWLAAGFSILLLMFYAYQTWKSTCGWEEIYVCAIEMVKVILEFFFEF
KNPSMLYLATGHRVQWLRYAEWLLTSPVILIHLSNLTGLSNDYSRRTMGLL
VSDIGTIVWGATSAMATGYVKVIFFCLGLCYGANTFFHAAKAYIEGYHTVP
KGRCRQVVTGMAWLFFVSWGMFPILFILGPEGFGVLSVYGSTVGHTIIDLM
SKNCWGLLGHYLRVLIHEHILIHGDIRKTTKLNIGGTEIEVETLVEDEAEA
GAVP.

In some embodiments, the channelrhodopsin is encoded by the nucleic acid sequence:

(SEQ ID NO: 16)
ATGGACTATGGCGGCGCTTTGTCTGCCGTCGGACGCGAACTTTTGTTCGTT
ACTAATCCTGTGGTGGTGAACGGGTCCGTCCTGGTCCCTGAGGATCAATGT
TACTGTGCCGGATGGATTGAATCTCGCGGCACGAACGGCGCTCAGACCGCG
TCAAATGTCCTGCAGTGGCTTGCAGCAGGATTCAGCATTTTGCTGCTGATG
TTCTATGCCTACCAAACCTGGAAATCTACATGCGGCTGGGAGGAGATCTAT
GTGTGCGCCATTGAAATGGTTAAGGTGATTCTCGAGTTCTTTTTTGAGTTT
AAGAATCCCTCTATGCTCTACCTTGCCACAGGACACCGGGTGCAGTGGCTG
CGCTATGCAGAGTGGCTGCTCACTTCTCCTGTCATCCTTATCCACCTGAGC
AACCTCACCGGCCTGAGCAACGACTACAGCAGGAGAACCATGGGACTCCTT
GTCTCAGACATCGGGACTATCGTGTGGGGGGCTACCAGCGCCATGGCAACC
GGCTATGTTAAAGTCATCTTCTTTTGTCTTGGATTGTGCTATGGCGCGAAC
ACATTTTTTCACGCCGCCAAAGCATATATCGAGGGTTATCATACTGTGCCA
AAGGGTCGGTGCCGCCAGGTCGTGACCGGCATGGCATGGCTGTTTTTCGTG
AGCTGGGGTATGTTCCCAATTCTCTTCATTTTGGGGCCCGAAGGTTTTGGC
GTCCTGAGCGTCTATGGCTCCACCGTAGGTCACACGATTATTGATCTGATG
AGTAAAAATTGTTGGGGGTTGTTGGGACACTACCTGCGCGTCCTGATCCAC
GAGCACATATTGATTCACGGAGATATCCGCAAAACCACCAAACTGAACATC
GGCGGAACGGAGATCGAGGTCGAGACTCTCGTCGAAGACGAAGCCGAGGCC
GGAGCCGTGCCA.

In some embodiments, the channelrhodopsin is VChR1, which can have the following amino acid sequence

(SEQ ID NO: 17)
MDYPVARSLIVRYPTDLGNGTVCMPRGQCYCEGWLRSRGTSIEKTIAITLQ
WVVFALSVACLGWYAYQAWRATCGWEEVYVALIEMMKSIIEAFHEFDSPAT
LWLSSGNGVVWMRYGEWLLTCPVLLIHLSNLTGLKDDYSKRTMGLLVSDVG
CIVWGATSAMCTGWTKILFFLISLSYGMYTYFHAAKVYIEAFHTVPKGICR
ELVRVMAWTFFVAWGMFPVLFLLGTEGFGHISPYGSAIGHSILDLIAKNMW
GVLGNYLRVKIHEHILLYGDIRKKQKITIAGQEMEVETLVAEEED.

In some embodiments, the channelrhodopsin is encoded by the nucleic acid sequence:

(SEQ ID NO: 18)
ATGGACTATCCTGTTGCTAGAAGCCTCATAGTTCGCTACCCAACCGACCTC
GGAAACGGCACCGTCTGCATGCCAAGAGGACAGTGTTACTGTGAAGGTTGG
CTTCGGAGTCGCGGCACTTCCATTGAAAAGACAATAGCAATTACTCTTCAG
TGGGTAGTCTTTGCTTTGTCAGTGGCTTGCCTGGGGTGGTATGCGTATCAA
GCGTGGCGAGCTACCTGCGGATGGGAGGAGGTTTACGTAGCCTTGATAGAA
ATGATGAAAAGCATCATCGAGGCCTTCCACGAGTTCGACAGCCCTGCAACA
CTGTGGCTGTCTTCAGGGAACGGCGTAGTTTGGATGCGGTATGGCGAATGG
CTCCTCACCTGCCCGGTCCTTCTGATCCATCTGAGCAACCTCACAGGCCTG
AAGGACGATTATAGCAAAAGGACTATGGGCCTGTTGGTTTCTGATGTGGGA
TGCATCGTGTGGGGCGCAACCAGCGCCATGTGTACGGGGTGGACGAAGATC
CTGTTCTTCCTCATCTCATTGAGCTATGGTATGTATACCTATTTTCATGCT
GCTAAAGTTTATATCGAAGCATTCCACACAGTTCCAAAAGGGATTTGTCGA
GAACTGGTCCGAGTGATGGCCTGGACATTCTTTGTGGCTTGGGGAATGTTT
CCAGTCCTGTTTCTGCTGGGCACGGAAGGATTCGGTCATATCAGCCCTTAT
GGATCTGCCATTGGGCACTCCATCCTCGACCTGATTGCAAAGAACATGTGG
GGTGTGCTGGGGAATTACCTGCGCGTCAAAATCCACGAGCACATCCTGTTG
TATGGCGACATCAGAAAGAAGCAGAAAATTACGATCGCCGGCCAAGAGATG
GAGGTTGAGACACTGGTGGCTGAAGAGGAGGAC.

In some embodiments, the channelrhodopsin is C1V1, which can have the following amino acid sequence:

(SEQ ID NO: 19)
MSRRPWLLALALAVALAAGSAGASTGSDATVPVATQDGPDYVFHRAHERML
FQTSYTLENNGSVICIPNNGQCFCLAWLKSNGTNAEKLAANILQWITFALS
ALCLMFYGYQTWKSTCGWEEIYVATIEMIKFIIEYFHEFDEPAVIYSSNGN
KTVWLRYAEWLLTCPVLLIHLSNLTGLKDDYSKRTMGLLVSDVGCIVWGAT
SAMCTGWTKILFFLISLSYGMYTYFHAAKVYIEAFHTVPKGICRELVRVMA
WTFFVAWGMFPVLFLLGTEGFGHISPYGSAIGHSILDLIAKNMWGVLGNYL
RVKIHEHILLYGDIRKKQKITIAGQEMEVETLVAEEED.

In some embodiments, the channelrhodopsin is encoded by the nucleic acid sequence:

(SEQ ID NO: 20)
ATGTCGCGGAGGCCATGGCTTCTTGCCCTAGCGCTGGCAGTGGCGCTGGCG
GCCGGCAGCGCAGGAGCCTCGACTGGCAGTGACGCGACGGTGCCGGTCGCG
ACTCAGGATGGCCCCGACTACGTTTTCCACCGTGCCCACGAGCGCATGCTC
TTCCAAACCTCATACACTCTTGAGAACAATGGTTCTGTTATTTGCATCCCG
AACAACGGCCAGTGCTTCTGCTTGGCTTGGCTTAAATCCAACGGAACAAAT
GCCGAGAAGTTGGCTGCCAACATTCTGCAGTGGATTACTTTTGCGCTTTCA
GCGCTCTGCCTGATGTTCTACGGCTACCAGACCTGGAAGTCTACTTGCGGC
TGGGAGGAGATTTACGTGGCCACGATCGAGATGATCAAGTTCATCATCGAG
TATTTCCATGAGTTTGACGAACCTGCGGTGATCTACTCATCCAACGGCAAC
AAGACCGTGTGGCTTCGTTACGCGGAGTGGCTGCTCACCTGCCCGGTCCTT
CTGATCCATCTGAGCAACCTCACAGGCCTGAAGGACGATTATAGCAAAAGG
ACTATGGGCCTGTTGGTTTCTGATGTGGGATGCATCGTGTGGGGCGCAACC
AGCGCCATGTGTACGGGGTGGACGAAGATCCTGTTCTTCCTCATCTCATTG
AGCTATGGTATGTATACCTATTTTCATGCTGCTAAAGTTTATATCGAAGCA
TTCCACACAGTTCCAAAAGGGATTTGTCGAGAACTGGTCCGAGTGATGGCC
TGGACATTCTTTGTGGCTTGGGGAATGTTTCCAGTCCTGTTTCTGCTGGGC
ACGGAAGGATTCGGTCATATCAGCCCTTATGGATCTGCCATTGGGCACTCC
ATCCTCGACCTGATTGCAAAGAACATGTGGGGTGTGCTGGGGAATTACCTG
CGCGTCAAAATCCACGAGCACATCCTGTTGTATGGCGACATCAGAAAGAAG
CAGAAAATTACGATCGCCGGCCAAGAGATGGAGGTTGAGACACTGGTGGCT
GAAGAGGAGGAC.

Reporter

mCherry can have the amino acid sequence:

(SEQ ID NO: 21)
VSKGEEDNMAIIKEFMRFKVHMEGSVNGHEFEIEGEGEGRPYEGTQTAKLK
VTKGGPLPFAWDILSPQFMYGSKAYVKHPADIPDYLKLSFPEGFKWERVMN
FEDGGVVTVTQDSSLQDGEFIYKVKLRGTNFPSDGPVMQKKTMGWEASSER
MYPEDGALKGEIKQRLKLKDGGHYDAEVKTTYKAKKPVQLPGAYNVNIKLD
ITSHNEDYTIVEQYERAEGRHSTGGMDELYK.

mCherry can be encoded by the nucleic acid sequence:

(SEQ ID NO: 22)
GTGAGCAAGGGCGAGGAGGATAACATGGCCATCATCAAGGAGTTCATGC
GCTTCAAGGTGCACATGGAGGGCTCCGTGAACGGCCACGAGTTCGAGAT
CGAGGGCGAGGGCGAGGGCCGCCCCTACGAGGGCACCCAGACCGCCAAG
CTGAAGGTGACCAAGGGTGGCCCCCTGCCCTTCGCCTGGGACATCCTGT
CCCCTCAGTTCATGTACGGCTCCAAGGCCTACGTGAAGCACCCCGCCGA
CATCCCCGACTACTTGAAGCTGTCCTTCCCCGAGGGCTTCAAGTGGGAG
CGCGTGATGAACTTCGAGGACGGCGGCGTGGTGACCGTGACCCAGGACT
CCTCCCTGCAGGACGGCGAGTTCATCTACAAGGTGAAGCTGCGCGGCAC
CAACTTCCCCTCCGACGGCCCCGTAATGCAGAAGAAGACCATGGGCTGG
GAGGCCTCCTCCGAGCGGATGTACCCCGAGGACGGCGCCCTGAAGGGCG
AGATCAAGCAGAGGCTGAAGCTGAAGGACGGCGGCCACTACGACGCTGA
GGTCAAGACCACCTACAAGGCCAAGAAGCCCGTGCAGCTGCCCGGCGCC
TACAACGTCAACATCAAGTTGGACATCACCTCCCACAACGAGGACTACA
CCATCGTGGAACAGTACGAACGCGCCGAGGGCCGCCACTCCACCGGCGG
CATGGACGAGCTGTACAAG.

eYFP can be encoded by the nucleic acid sequence:

(SEQ ID NO: 23)
ATGGTGAGCAAGGGCGAGGAGCTGTTCACCGGGGTGGTGCCCATCCTGG
TCGAGCTGGACGGCGACGTAAACGGCCACAAGTTCAGCGTGTCCGGCGA
GGGCGAGGGCGATGCCACCTACGGCAAGCTGACCCTGAAGTTCATCTGC
ACCACCGGCAAGCTGCCCGTGCCCTGGCCCACCCTCGTGACCACCTTCG
GCTACGGCCTGCAGTGCTTCGCCCGCTACCCCGACCACATGAAGCAGCA
CGACTTCTTCAAGTCCGCCATGCCCGAAGGCTACGTCCAGGAGCGCACC
ATCTTCTTCAAGGACGACGGCAACTACAAGACCCGCGCCGAGGTGAAGT
TCGAGGGCGACACCCTGGTGAACCGCATCGAGCTGAAGGGCATCGACTT
CAAGGAGGACGGCAACATCCTGGGGCACAAGCTGGAGTACAACTACAAC
AGCCACAACGTCTATATCATGGCCGACAAGCAGAAGAACGGCATCAAGG
TGAACTTCAAGATCCGCCACAACATCGAGGACGGCAGCGTGCAGCTCGC
CGACCACTACCAGCAGAACACCCCCATCGGCGACGGCCCCGTGCTGCTG
CCCGACAACCACTACCTGAGCTACCAGTCCGCCCTGAGCAAAGACCCCA
ACGAGAAGCGCGATCACATGGTCCTGCTGGAGTTCGTGACCGCCGCCGG
GATCACTCTCGGCATGGACGAGCTGTACAAGTAA.

dTomado can be encoded by the nucleic acid sequence:

(SEQ ID NO: 24)
ATGGTGAGCAAGGGCGAGGAGGTCATCAAAGAGTTCATGCGCTTCAAGG
TGCGCATGGAGGGCTCCATGAACGGCCACGAGTTCGAGATCGAGGGCGA
GGGCGAGGGCCGCCCCTACGAGGGCACCCAGACCGCCAAGCTGAAGGTG
ACCAAGGGCGGCCCCCTGCCCTTCGCCTGGGACATCCTGTCCCCCCAGT
TCATGTACGGCTCCAAGGCGTACGTGAAGCACCCCGCCGACATCCCCGA
TTACAAGAAGCTGTCCTTCCCCGAGGGCTTCAAGTGGGAGCGCGTGATG
AACTTCGAGGACGGCGGTCTGGTGACCGTGACCCAGGACTCCTCCCTGC
AGGACGGCACGCTGATCTACAAGGTGAAGATGCGCGGCACCAACTTCCC
CCCCGACGGCCCCGTAATGCAGAAGAAGACCATGGGCTGGGAGGCCTCC
ACCGAGCGCCTGTACCCCCGCGACGGCGTGCTGAAGGGCGAGATCCACC
AGGCCCTGAAGCTGAAGGACGGCGGCCACTACCTGGTGGAGTTCAAGAC
CATCTACATGGCCAAGAAGCCCGTGCAACTGCCCGGCTACTACTACGTG
GACACCAAGCTGGACATCACCTCCCACAACGAGGACTACACCATCGTGG
AACAGTACGAGCGCTCCGAGGGCCGCCACCACCTGTTCCTGTACGGCAT
GGACGAGCTGTACAAGTAA.

Promoters

Suitable promoters include CMV, cfos, hNSE, CgA, CAG, INSM1, hSyn, and CaMKII. For example, the CMV promoter can have the nucleic acid sequence:

(SEQ ID NO: 25)
TAGTAATCAATTACGGGGTCATTAGTTCATAGCCCATATATGGAGTTCC
GCGTTACATAACTTACGGTAAATGGCCCGCCTGGCTGACCGCCCAACGA
CCCCCGCCCATTGACGTCAATAATGACGTATGTTCCCATAGTAACGCCA
ATAGGGACTTTCCATTGACGTCAATGGGTGGACTATTTACGGTAAACTG
CCCACTTGGCAGTACATCAAGTGTATCATATGCCAAGTACGCCCCCTAT
TGACGTCAATGACGGTAAATGGCCCGCCTGGCATTATGCCCAGTACATG
ACCTTATGGGACTTTCCTACTTGGCAGTACATCTACGTATTAGTCATCG
CTATTACCATGGTGATGCGGTTTTGGCAGTACATCAATGGGCGTGGATA
GCGGTTTGACTCACGGGGATTTCCAAGTCTCCACCCCATTGACGTCAAT
GGGAGTTTGTTTTGGCACCAAAATCAACGGGACTTTCCAAAATGTCGTA
ACAACTCCGCCCCATTGACGCAAATGGGCGGTAGGCGTGTACGGTGGGA
GGTCTATATAAGCAGAGCTCTCTGGCTAACTAGAGAACCCACTGCTTAC
TGGCTTATCGAAAT.

The cfos promoter can have the nucleic acid sequence:

(SEQ ID NO: 26)
ACGCGTAAGCTTTCCTTTAGGAACAGAGGCTTCGAGCCTTTAAGGCTGC
GTACTTGCTTCTCCTAATACCAGAGACTCAAAAAAAAAAAAAAAGTTCC
AGATTGCTGGACAATGACCCGGGTCTCATCCCTTGACCCTGGGAACCGG
GTCCACATTGAATCAGGTGCGAATGTTCGCTCGCCTTCTCTGCCTTTCC
CGCCTCCCCTCCCCCGGCCGCGGCCCCGGTTCCCCCCCTGCGCTGCACC
CTCAGAGTTGGCTGCAGCCGGCGAGCTGTTCCCGTCAATCCCTCCCTCC
TTTACACAGGATGTCCATATTAGGACATCTGCGTCAGCAGGTTTCCACG
GCCGGTCCCTGTTGTTCTGGGGGGGGGACCATCTCCGAAATCCTACACG
CGGAAGGTCTAGGAGACCCCCTAAGATCCCAAATGTGAACACTCATAGG
TGAAAGATGTATGCCAAGACGGGGGTTGAAAGCCTGGGGCGTAGAGTTG
ACGACAGAGCGCCCGCAGAGGGCCTTGGGGCGCGCTTCCCCCCCCTTCC
AGTTCCGCCCAGTGACGTAGGAAGTCCATCCATTCACAGCGCTTCTATA
AAGGCGCCAGCTGAGGCGCCTACTACTCCAACCGCGACTGCAGCGAGCA
ACTGAGAAGACTGGATAGAGCCGGCGGTTCCGCGAACGAGCAGTGACCG
CGCTCCCACCCAGCTCTGCTCTGCAGCTCCCACCAGTGTCTACCCCTGG
ACCCCTTGCCGGGCTTTCCCCAAACTTCGACCATGATGTTCTCGGGTTT
CAACGCCGACTACGAGGCGTCATCCTCCCGCTGCAGTAGCGCCTCCCCG
GCCGGGGACAGCCTTTCCTACTACCATTCCCCAGCCGACTCCTTCTCCA
GCATGGGCTCTCCTGTCAACACACAGGTGAGTTTGGCTTTGTGTAGCCG
CCAGGTCCGCGCTGAGGGTCGCCGTGGAGGAGACACTGGGGTGTGACTC
GCAGGGGCGGGGGGGTCTTCCTTTTTCGCTCTGGAGGGAGACTGGCGCG
GTCAGAGCAGCCTTAGCCTGGGAACCCAGGACTTGTCTGAGCGCGTGCA
CACTTGTCATAGTAAGACTTAGTGACCCCTTCCCGCGCGGCAGGTTTAT
TCTGAGTGGCCTGCCTGCATTCTTCTCTCGGCCGACTTGTTTCTGAGAT
CAGCCGGGGCCAACAAGTCTCGAGCAAAGAGTCGCTAACTAGAGTTTGG
GAGGCGGCAAACCGCGGCAATCCCCCCTCCCGGGGCAGCCTGGAGCAGG
GAGGAGGGAGGAGGGAGGAGGGTGCTGCGGGCGGGTGTGTAAGGCAGTT
TCATTGATAAAAAGCGAGTTCATTCTGGAGACTCCGGAGCAGCGCCTGC
GTCAGCGCAGACGTCAGGGATATTTATAACAAACCCCCTTTCGAGCGAG
TGATGCCGAAGGGATAACGGGAACGCAGCAGTAGGATGGAGGGGAAAGG
CTGCGCTGCGGAATTCAAGGGAGGATATTGGGAGAGCTTTTATCTCCGA
TGAGGTGCATACAGGAAGACATAAGCAGTCTCTGACCGGAATGCTTCTC
TCTCCCTGCTTCATGCGACACTAGGGCCACTTGCTCCACCTGTGTCTGG
AACCTCCTCGCTCACCTCCGCTTTCCTCTTTTTGTTTTGTTTCAGGACT
TTTGCGCAGATCTCTC.

The hNSE promoter can have the nucleic acid sequence:

(SEQ ID NO: 27)
TGTATGCAGCTGGACCTAGGAGAGAAGCAGGAGAGGAAGATCCAGCACA
AAAAATCTGAAGCTAAAAACAGGACACAGAGATGGGGGAAGAAAAGAGG
GCAGAGTGAGGCAAAAAGAGACTGAAGAGATGAGGGTGGCCGCCAGGCA
CTTTAGATAGGGGAGAGGCTTTATTTACCTCTGTTTGTTTTTTTTTTTT
TTTTTTTTTTTTTTTTGCGAGGTAGTCTTGCTTAGTCTCCAGGCTGGAG
TGCAGTGGCACAATCTCAGCTCACTGCAACTTCCACCTCCTGGGTTCAA
GCAATTCTCCTGCCTCAGCCTCCCGAGTAGCTGGGACTACAGGCGCATG
CAACCGCGCCTGGCTAATTTTTGTATTTTTAGTAGAAACGGGGTTTCAC
CACGTTAGCCAGGATGGTCTGGATCTCCTGACCTCGTGATCTGCCCGCC
TCCGCCTTCCAAAGTGCTGGGATTACAGGGGTGAGCCACAGCGCCTGGT
CCCTATTTACTTCTGTCTTCTACCTCCAGGAGATCAAAGACGCTGGCCT
TCAGACCTGATCAGACTCCCAGGGGCAGCCACCACATGTATGACAGAGA
ACAGAGGATGCCTGTTTTTGCCCCAAAGCTGGAAATTCATCACAACCTG
AGGCCCAGGATCTGCTCTGTGCCGGTCCTCTGGGCAGTGTGGGGTGCAG
AATGGGGTGCCTAGGCCTGAGCGTTGCCTGGAGCCTAGGCCGGGGGCCG
CCCTCGGGCAGGCGTGGGTGAGAGCCAAGACCGCGTGGGCCGCGGGGTG
CTGGTAGGAGTGGTTGGAGAGACTTGCGAAGGCGGCTGGGGTGTTCGGA
TTTCCAATAAAGAAACAGAGTGATGCTCCTGTGTCTGACCGGGTTTGTG
AGACATTGAGGCTGTCTTGGGCTTCACTGGCAGTGTGGGCCTTCGTACC
CGGGCTACAGGGGTGCGGCTCTGCCTGTTACTGTCGAGTGGGTCGGGCC
GTGGGTATGAGCGCTTGTGTGCGCTGGGGCCAGGTCGTGGGTGCCCCCA
CCCTTCCCCCATCCTCCTCCCTTCCCCACTCCACCCTCGTCGGTCCCCC
ACCCGCGCTCGTACGTGCGCCTCCGCCGGCAGCTCCTGACTCATCGGGG
GCTCCGGGTCACATGCGCCCGCGCGGCCCTATAGGCGCCTCCTCCGCCC
GCCGCCCGGGAGCCGCAGCCGCCGCCGCCACTGCCACTCCCGCTCTCTC
AGCGCCGCCGTCGCCACCGCCACCGCCACCGCCACTACCACCG.

The CgA promoter can have the nucleic acid sequence:

(SEQ ID NO: 28)
AAGAAAAGGTGAATGGTTGGGATGCATACTGGAAGGAAACAACGGAAATC
TGAAAAGGTGTAAGAACCTAAACAAATTTGTTTATCACAGAAAATAAATC
ACAAAACAACTTTGCGTTCTTTGGCAAGTTTCTTTATGTTAAACAAGAAT
TGCTTTTTGCATCACATAGATCTTCTAAACTCTTTGTTGAAGAGGTCCTT
GGTAGTCTGTATCTAAGCCAGTTCCTTACGGAAGTGGCACTGAGCGGAGT
AGATAAAGATAGGAACTTTTGAAGGGTCATAATCTCTGTGTGCAAAAAAG
AAGCCACAGTAGTCTGAAGAGCTGTGCAGGTTTTAGGGTGACACTGGGTT
GGGAACCTTGGAGCTAAGTGTCCCACACCTGGCAAGCCATGACATACATA
TTTTCTGTTCAGGCAGAAACTGAGCTTTACAAAAGTGAAATGAGAAAAAA
AAAAAAACCAAAAACCAGGCACGTATATTGAGAACCATTCAGTCCTTCTT
AGAATTGCCTCATACCTTTCTCATGCATCTTTATTAAATTCAGATGCAAA
TTAATTTTAGAAAAGTCTAAATAGGTGTGTGTTTTATTTTTCTGTTTCCT
AATTAAATAGTGGTATAAGCCTGGAAATGCTCTATATCTATTTTCGGAAA
TCTATAGCTCTTGTTTAGGTAAATATCAGGTACTTAGCTAATTAAATGTC
TCTTGTTTATAGGAAAGTGTCAGCTTTCAGGATGTTATGTGTATGGCTCA
ATAAAATTACGTACAAAGTGACAGCGTACTCTCTTTTCATGGGCTGACCT
TGTCGTCACCATCACCTGAAAATGGCTCCAAACAAAAATGACCTAAGGGT
TGAAACAAGATAAGATCAAATTGACGTCATGGTAAAAATTGACGTCATGG
TAATTACACCAAGTACCCTTCAATCATTGGATGGAATTTCCTGTTGATCC
CAGGGCTTAGATGCAGGTGGAAACACTCTGCTGGTATAAAAGCAGGTGAG
GACTTCATTAACTGCAGTTACTGAGAACTCATAAGACGAAGCTAAAATCC
CTCTTCGGATCCAC.

The CAG promoter can have the nucleic acid sequence:

(SEQ ID NO: 29)
CCATTGACGTCAATAATGACGTATGTTCCCATAGTAACGCCAATAGGGAC
TTTCCATTGACGTCAATGGGTGGAGTATTTACGGTAAACTGCCCACTTGG
CAGTACATCAAGTGTATCATATGCCAAGTACGCCCCCTATTGACGTCAAT
GACGGTAAATGGCCCGCCTGGCATTATGCCCAGTACATGACCTTATGGGA
CTTTCCTACTTGGCAGTACATCTACGTATTAGTCATCGCTATTACCATGG
TCGAGGTGAGCCCCACGTTCTGCTTCACTCTCCCCATCTCCCCCCCCTCC
CCACCCCCAATTTTGTATTTATTTATTTTTTAATTATTTTGTGCAGCGAT
GGGGGCGGGGGGGGGGGGGGGGCGCGCGCCAGGCGGGGCGGGGCGGGGCG
AGGGGCGGGGCGGGGCGAGGCGGAGAGGTGCGGCGGCAGCCAATCAGAGC
GGCGCGCTCCGAAAGTTTCCTTTTATGGCGAGGCGGCGGCGGCGGCGGCC
CTATAAAAAGCGAAGCGCGCGGCGGGCGGGAGTCGCTGCGACGCTGCCTT
CGCCCCGTGCCCCGCTCCGCCGCCGCCTCGCGCCGCCCGCCCCGGCTCTG
ACTGACCGCGTTACTCCCACAGGTGAGCGGGCGGGACGGCCCTTCTCCTC
CGGGCTGTAATTAGCGCTTGGTTTAATGACGGCTTGTTTCTTTTCTGTGG
CTGCGTGAAAGCCTTGAGGGGCTCCGGGAGGGCCCTTTGTGCGGGGGGAG
CGGCTCGGGGCTGTCCGCGGGGGGACGGCTGCCTTCGGGGGGGACGGGGC
AGGGCGGGGTTCGGCTTCTGGCGTGTGACCGGCGGCTCTAGAGCCTCTGC
TAACCATGTTCATGCCTTCTTCTTTTTCCTACAGCTCCTGGGCAACGTGC
TGGTTATTGTGCTGTCTCATCATTTTGGCAAAGAATT.

The INSM1 promoter can have the nucleic acid sequence:

CCAATTCCTTCTCAAACTCGAAAGAAACCTTCTCTAGCCCCGTGGGCGCG
CGGAGGCTGCGAGCACAAACATCGCCCTCGGCCACTGCCAGAAGGCCGGG
CCCCCTGTCCACACTTGGAACCCCGGGGAACCCTTTTGCTTGGCCTCTTG
GGTCCAGCGGCCCATCCGTCCAA

(SEQ ID NO: 30)
GGTCCGGGCGGAGGCCGTCCGGACCCTGCTGCTCTCTCGGATTCTTGTTT
ATTTCCCAAACACCACGCGGAGCCACTGCGCCTCCGCAACGATCTCCCCC
GCACCGCCCCGGCGCGCCCCCGCCCCCACCCAATCAGCGCGCACAACTTC
CCCCTCGGCTCCGGCTCGCGGATTGAACCCTCCTGACATATTTGGGGCCA
TTCTTCTCCTTTGTTGCTATTTTGCTAGCGACCCGCGGGTAATCCCCGCG
CGGGAGGGGGGCGTGCATTGTCGCGCTGATGGACGGGCCCATTTGGCGGC
TCCGCGCCCCCCGGAGGAGAGACACAAAGCCCAGGCACGTGCGCCTCCCC
ATAGAGAAGCAGCAGACCGTGAAGGGAGGCGGGGCCGGGCGTGTGCCTGG
ACCGGGCGGGGCGGCGGCGCCGGGCGGGGCGACCAGGGGCGCGCGCGGGG
GCCCCGCGCCCTCAGGTACATCTGCCGCACCTACCGGGCGACCCCCGAGT
CCCGGCCCCCTTTTGGCCGCCCCATCGCCCTCCCACCCTGCCAGGCTGAG
GAGCTGCGGACGCGCTGATTGGCTCCAGGGGAAGCGGGAGGCGAGAACAA
TGGCCCCCTCCCCCCGTTAAAAGGGAGCGGCTGCCGGGCCCGGGGACAGG
GACGCGCGTGCAGGGCGCAGAGCTGGGCCGAGCCGTCGCCGGCGCCACGC
GAGTCCCGCAGCCGCCGCGCCCGGGCAATGGGCCGGGGGCACTGAGGGCC
GCCGGGGCCGAGCGCGGAGGGGGGACCGAGCCAGTGCCGTGCCCTCGGGC
CGCGCCAACATGCCCCGCGGCTTCCTGGTGAAGCGCAGCAA.

The hSyn promoter can have the nucleic acid sequence:

(SEQ ID NO: 31)
CTGCGCTCTCAGGCACGACACGACTCCTCCGCTGCCCACCGCAGACTGAG
GCAGCGCTGAGTCGCCGGCGCCGCAGCGCAGATGGTCGCGCCCGTGCCCC
CCTATCTCGCGCCTCGCGTGGTGCGGTCCGGCTGGGCCGGCGGCGGCGCG
GACGCGACCAAGGTGGCCGGGAAGGGGAGTTTGCGGGGGACCGGCGAGTG
ACGTCAGCGCGCCTTCAGTGCTGAGGCGGCGGTGGCGCGCGCCGCCAGGC
GGGGGCGAAGGCACTGTCCGCGGTGCTGAAGCTGGCAGTGCGCACGCGCC
TCGCCGCATCCTGTTTCCCCTCCCCCTCTCTGATAGGGGATGCGCAATTT
GGGGAATGGGGGTTGGGTGCTTGTCCAGTGGGTCGGGGTCGGTCGTCAGG
TAGGCACCCCCACCCCGCCTCATCCTGGTCCTAAAACCCACTTGCACT.

The CaMKII promoter can have the nucleic acid sequence:

(SEQ ID NO: 32)
TTAACATTATGGCCTTAGGTCACTTCATCTCCATGGGGTTCTTCTTCTGA
TTTTCTAGAAAATGAGATGGGGGTGCAGAGAGCTTCCTCAGTGACCTGCC
CAGGGTCACATCAGAAATGTCAGAGCTAGAACTTGAACTCAGATTACTAA
TCTTAAATTCCATGCCTTGGGGGCATGCAAGTACGATATACAGAAGGAGT
GAACTCATTAGGGCAGATGACCAATGAGTTTAGGAAAGAAGAGTCCAGGG
CAGGGTACATCTACACCACCCGCCCAGCCCTGGGTGAGTCCAGCCACGTT
CACCTCATTATAGTTGCCTCTCTCCAGTCCTACCTTGACGGGAAGCACAA
GCAGAAACTGGGACAGGAGCCCCAGGAGACCAAATCTTCATGGTCCCTCT
GGGAGGATGGGTGGGGAGAGCTGTGGCAGAGGCCTCAGGAGGGGCCCTGC
TGCTCAGTGGTGACAGATAGGGGTGAGAAAGCAGACAGAGTCATTCCGTC
AGCATTCTGGGTCTGTTTGGTACTTCTTCTCACGCTAAGGTGGCGGTGTG
ATATGCACAATGGCTAAAAAGCAGGGAGAGCTGGAAAGAAACAAGGACAG
AGACAGAGGCCAAGTCAACCAGACCAATTCCCAGAGGAAGCAAAGAAACC
ATTACAGAGACTACAAGGGGGAAGGGAAGGAGAGATGAATTAGCTTCCCC
TGTAAACCTTAGAACCCAGCTGTTGCCAGGGCAACGGGGCAATACCTGTC
TCTTCAGAGGAGATGAAGTTGCCAGGGTAACTACATCCTGTCTTTCTCAA
GGACCATCCCAGAATGTGGCACCCACTAGCCGTTACCATAGCAACTGCCT
CTTTGCCCCACTTAATCCCATCCCGTCTGTTAAAAGGGCCCTATAGTTGG
AGGTGGGGGAGGTAGGAAGAGCGATGATCACTTGTGGACTAAGTTTGTTC
GCATCCCCTTCTCCAACCCCCTCAGTACATCACCCTGGGGGAACAGGGTC
CACTTGCTCCTGGGCCCACACAGTCCTGCAGTATTGTGTATATAAGGCCA
GGGCAAAGAGGAGCAGGTTTTAAAGTGAAAGGCAGGCAGGTGTTGGGGAG
GCAGTTACCGGGGCAACGGGAACAGGGCGTTTCGGAGGTGGTTGCCATGG
GGACCTGGATGCTGACGAAGGCTCGCGAGGCTGTGAGCAGCCACAGTGCC
CTGCTCAGAAGCCCCAAGCTCGTCAGTCAAGCCGGTTCTCCGTTTGCACT
CAGGAGCACGGGCAGGCGAGTGGCCCCTAGTTCTGGGGGCAGC.

Luciferase

Suitable luciferase proteins include hGluc, hRLuc, M23Gluc, sbGLuc, and NLuc. For example, the luciferase protein can be hGluc and have the amino acid sequence:

(SEQ ID NO: 33)
MGVKVLFALICIAVAEAKPTENNEDFNIVAVASNFATTDLDADRGKLPGK
KLPLEVLKEMEANARKAGCTRGCLICLSHIKCTPKMKKFIPGRCHTYEGD
KESAQGGIGEAIVDIPEIPGFKDLEPMEQFIAQVDLCVDCTTGCLKGLAN
VQCSDLLKKWLPQRCATFASKIQGQVDKIKGAGGD.

hGluc can be encoded by the nucleic acid sequence:

(SEQ ID NO: 34)
ATGGGAGTCAAAGTTCTGTTTGCCCTGATCTGCATCGCTGTGGCCGAGG
CCAAGCCCACCGAGAACAACGAAGACTTCAACATCGTGGCCGTGGCCAG
CAACTTCGCGACCACGGATCTCGATGCTGACCGCGGGAAGTTGCCCGGC
AAGAAGCTGCCGCTGGAGGTGCTCAAAGAGATGGAAGCCAATGCCCGGA
AAGCTGGCTGCACCAGGGGCTGTCTGATCTGCCTGTCCCACATCAAGTG
CACGCCCAAGATGAAGAAGTTCATCCCAGGACGCTGCCACACCTACGAA
GGCGACAAAGAGTCCGCACAGGGCGGCATAGGCGAGGCGATCGTCGACA
TTCCTGAGATTCCTGGGTTCAAGGACTTGGAGCCCATGGAGCAGTTCAT
CGCACAGGTCGATCTGTGTGTGGACTGCACAACTGGCTGCCTCAAAGGG
CTTGCCAACGTGCAGTGTTCTGACCTGCTCAAGAAGTGGCTGCCGCAAC
GCTGTGCGACCTTTGCCAGCAAGATCCAGGGCCAGGTGGACAAGATCAA
GGGGGCCGGTGGTGAC.

The luciferase protein can be hRluc and have the amino acid sequence:

(SEQ ID NO: 35)
MASKVYDPEQRKRMITGPQVWVARCKQMNVLDSFINYYDSEKHAENAVI
FLHGNAASSYLWRHVVPHIEPVARCIIPDLIGMGKSGKSGNGSYRLLDH
YKYLTAWFELLNLPKKIIFVGHDWGACLAFHYSYEHQDKIKAIVHAESV
VDVIESWDEWPDIEEDIALIKSEEGEKMVLENNFFVETMLPSKIMRKLE
PEEFAAYLEPFKEKGEVRRPTLSWPREIPLVKGGKPDVVQIVRNYNAYL
RASDDL.

hRluc can be encoded by the nucleic acid sequence:

(SEQ ID NO: 36)
ATGGCTTCCAAGGTGTACGACCCCGAGCAACGCAAACGCATGATCACTG
GGCCTCAGTGGTGGGCTCGCTGCAAGCAAATGAACGTGCTGGACTCCTT
CATCAACTACTATGATTCCGAGAAGCACGCCGAGAACGCCGTGATTTTT
CTGCATGGTAACGCTGCCTCCAGCTACCTGTGGAGGCACGTCGTGCCTC
ACATCGAGCCCGTGGCTAGATGCATCATCCCTGATCTGATCGGAATGGG
TAAGTCCGGCAAGAGCGGGAATGGCTCATATCGCCTCCTGGATCACTAC
AAGTACCTCACCGCTTGGTTCGAGCTGCTGAACCTTCCAAAGAAAATCA
TCTTTGTGGGCCACGACTGGGGGGCTTGTCTGGCCTTTCACTACTCCTA
CGAGCACCAAGACAAGATCAAGGCCATCGTCCATGCTGAGAGTGTCGTG
GACGTGATCGAGTCCTGGGACGAGTGGCCTGACATCGAGGAGGATATCG
CCCTGATCAAGAGCGAAGAGGGCGAGAAAATGGTGCTTGAGAATAACTT
CTTCGTCGAGACCATGCTCCCAAGCAAGATCATGCGGAAACTGGAGCCT
GAGGAGTTCGCTGCCTACCTGGAGCCATTCAAGGAGAAGGGCGAGGTTA
GACGGCCTACCCTCTCCTGGCCTCGCGAGATCCCTCTCGTTAAGGGAGG
CAAGCCCGACGTCGTCCAGATTGTCCGCAACTACAACGCCTACCTTCGG
GCCAGCGACGATCTGCC.

The luciferase protein can be M23Gluc and have the amino acid sequence:

(SEQ ID NO: 37)
MGVKVLFALICIAVAEAKPTENNEDFNIVAVASNFATTDLDADRGKLPG
EKLPLEVLKELEANARKAGCTRGCLICLSHIKCTPKMKKFIPGRCHTYE
GDKESAQGGIGEAIDDIPEIPGFKDLEPIEQFIAQVDLCVDCTTGCLKG
LANVQCSDLLKKWLPQRCATFASKIQGQVDKIKGAGDD.

M23Gluc can be encoded by the nucleic acid sequence:

(SEQ ID NO: 38)
ATGGGAGTCAAAGTTCTGTTTGCCCTGATCTGCATCGCTGTGGCCGAGG
CCAAGCCCACCGAGAACAACGAAGACTTCAACATCGTGGCCGTGGCCAG
CAACTTCGCGACCACGGATCTCGATGCTGACCGCGGGAAGTTGCCCGGC
GAGAAGCTGCCGCTGGAGGTGCTCAAAGAGCTGGAAGCCAATGCCCGGA
AAGCTGGCTGCACCAGGGGCTGTCTGATCTGCCTGTCCCACATCAAGTG
CACGCCCAAGATGAAGAAGTTCATCCCAGGACGCTGCCACACCTACGAA
GGCGACAAAGAGTCCGCACAGGGCGGCATAGGCGAGGCGATCGACGACA
TTCCTGAGATTCCTGGGTTCAAGGACTTGGAGCCCATCGAGCAGTTCAT
CGCACAGGTCGATCTGTGTGTGGACTGCACAACTGGCTGCCTCAAAGGG
CTTGCCAACGTGCAGTGTTCTGACCTGCTCAAGAAGTGGCTGCCGCAAC
GCTGTGCGACCTTTGCCAGCAAGATCCAGGGCCAGGTGGACAAGATCAA
GGGGGCCGGTGATGAC.

The luciferase protein can be sbGluc and have the amino acid sequence:

(SEQ ID NO: 39)
MGVKVLFALICIAVAEAKPTENNEDFNIVAVASNFATTDLDADRGKLPG
KKLPLEVLKELEANARKAGCTRGCLICLSHIKCTPKMKKFIPGRCHTYE
GDKESAQGGIGEAIVDIPEIPGFKDLEPLEQFIAQVDLCVDCTTGCLKG
LANVQCSDLLKKWLPQRCATFASKIQGQVDKIKGAGGD.

sbGluc can be encoded by the nucleic acid sequence:

(SEQ ID NO: 40)
ATGGGAGTCAAAGTTCTGTTTGCCCTGATCTGCATCGCTGTGGCCGAGG
CCAAGCCCACCGAGAACAACGAAGACTTCAACATCGTGGCCGTGGCCAG
CAACTTCGCGACCACGGATCTCGATGCTGACCGCGGGAAGTTGCCCGGC
AAGAAGCTGCCGCTGGAGGTGCTCAAAGAGCTGGAAGCCAATGCCCGGA
AAGCTGGCTGCACCAGGGGCTGTCTGATCTGCCTGTCCCACATCAAGTG
CACGCCCAAGATGAAGAAGTTCATCCCAGGACGCTGCCACACCTACGAA
GGCGACAAAGAGTCCGCACAGGGCGGCATAGGCGAGGCGATCGTCGACA
TTCCTGAGATTCCTGGGTTCAAGGACTTGGAGCCCCTGGAGCAGTTCAT
CGCACAGGTCGATCTGTGTGTGGACTGCACAACTGGCTGCCTCAAAGGG
CTTGCCAACGTGCAGTGTTCTGACCTGCTCAAGAAGTGGCTGCCGCAAC
GCTGTGCGACCTTTGCCAGCAAGATCCAGGGCCAGGTGGACAAGATCAA
GGGGGCCGGTGGTGAC.

The luciferase protein can be Nluc and have the amino acid sequence:

(SEQ ID NO: 41)
MVFTLEDFVGDWRQTAGYNLDQVLEQGGVSSLFQNLGVSVTPIQRIVLS
GENGLKIDIHVIIPYEGLSGDQMGQIEKIFKVVYPVDDHHFKVILHYGT
LVIDGVTPNMIDYFGRPYEGIAVFDGKKITVTGTLWNGNKIIDERLINP
DGSLLFRVTINGVTGWRLCERILA.

Nluc can be encoded by the nucleic acid sequence:

(SEQ ID NO: 42)
ATGGTCTTCACACTCGAAGATTTCGTTGGGGACTGGCGACAGACAGCCG
GCTACAACCTGGACCAAGTCCTTGAACAGGGAGGTGTGTCCAGTTTGTT
TCAGAATCTCGGGGTGTCCGTAACTCCGATCCAAAGGATTGTCCTGAGC
GGTGAAAATGGGCTGAAGATCGACATCCATGTCATCATCCCGTATGAAG
GTCTGAGCGGCGACCAAATGGGCCAGATCGAAAAAATTTTTAAGGTGGT
GTACCCTGTGGATGATCATCACTTTAAGGTGATCCTGCACTATGGCACA
CTGGTAATCGACGGGGTTACGCCGAACATGATCGACTATTTCGGACGGC
CGTATGAAGGCATCGCCGTGTTCGACGGCAAAAAGATCACTGTAACAGG
GACCCTGTGGAACGGCAACAAAATTATCGACGAGCGCCTGATCAACCCC
GACGGCTCCCTGCTGTTCCGAGTAACCATCAACGGAGTGACCGGCTGGC
GGCTGTGCGAACGCATTCTGGCG.

Linker

In some embodiments, the linker has the nucleic acid sequence:

(P2A, SEQ ID NO: 43)
GAGGGCAGGGGAAGTCTTCTAACATGCGGGGACGTGGAGGAAAATCCCG
GCCCC.

In some embodiments, the linker has the nucleic acid sequence:

(T2A, SEQ ID NO: 44)
GGATCAGGCAGCGGCGCCACGAACTTCTCTCTGTTAAAGCAAGCAGGAG
ACGTGGAAGAAAACCCCGGTCCC.

Leading Sequence

The inner mitochondrial membrane-mitochondrial localization signal (IMM-MLS) is in some embodiments a leading sequence from a mitochondrial inner membrane protein. For example, the mitochondrial inner membrane protein can be selected from ABCB10, ABCB140, Cytochrome C, and huROMK.

The outer mitochondrial membrane-mitochondrial localization signal (OMM-MLS) is in some embodiments a leading sequence from a mitochondrial outer membrane protein. For example, the mitochondrial outer membrane protein can be OMA25 or TOM20.

For example, the OMM-MLS can be OMA25, which has the nucleic acid sequence:

(SEQ ID NO: 45)
ATGCGAGGCGACGGAGAGCCGAGTGGAGTTCCTGTAGCTGTGGTGCTGC
TGCCAGTGTTTGCCCTTACCCTGGTAGCAGTTTGGGCCTTCGTGAGATA
CCGAAAGCAGCTC.

In some embodiments, the OMM-MLS can be TOM20, which has the nucleic acid sequence:

(SEQ ID NO: 46)
ATGGTGGGCCGGAACAGCGCCATCGCCGCGGGGCTGTGCGGTGCCCTCT
TCATAGGGTACTGCATCTACTTTGACCGCAAAAGGCGAGGTGACCCCAA
CTTCAAGGGGCTAGCGCTACCGG.

In some embodiments, the IMM-MLS can be ABCB10(140), which has the nucleic acid sequence:

(SEQ ID NO: 47)
ATGCGCGCCCCTTCTGCTAGGGCGCTACTGCTGATTCCGCGTCGGGGCC
CTGCCGTGCGAGCGTGGGCCCCGGCCGTCTCCTCTCGGATATGGCTGGC
TTCTGAATGGACCCCGCTCGTACGCGCGTGGACCTCTCTGATCCACAAG
CCGGGTTCGGGCCTCCGCTTTCCCGCGCCCCTATCCGGGCTGCCTGGCG
GCGTGGGGCAGTGGGCCACCTCCTCGGGGGCCCGCAGGTGCTGGGTGCT
GGCAGGACCCCGCGCCGCACATCCCCTGTTCGCCAGGCTCCAGGGTGCA
GCTGCCACCGGTGTGCGAGACCTTGGGAACGACTCGCAGCGGCGTCCCG
CGGCGACCGGGCGCTCAGAAGTATGGAAGCTCCTAGGGCTGGTGCGCCC
CGAGCGCGGGAGACTGTCAGCTGCAGTT.

In some embodiments, the IMM-MLS can be ABCB10(105), which has the nucleic acid sequence:

(SEQ ID NO: 48)
ATGCGCGCCCCTTCTGCTAGGGCGCTACTGCTGATTCCGCGTCGGGGCC
CTGCCGTGCGAGCGTGGGCCCCGGCCGTCTCCTCTCGGATATGGCTGGC
TTCTGAATGGACCCCGCTCGTACGCGCGTGGACCTCTCTGATCCACAAG
CCGGGTTCGGGCCTCCGCTTTCCCGCGCCCCTATCCGGGCTGCCTGGCG
GCGTGGGGCAGTGGGCCACCTCCTCGGGGGCCCGCAGGTGCTGGGTGCT
GGCAGGACCCCGCGCCGCACATCCCCTGTTCGCCAGGCTCCAGGGTGCA
GCTGCCACCGGTGTGCGAGAC.

In some embodiments, the IMM-MLS can be Cytochrome C MLS (mito), which has the nucleic acid sequence:

(SEQ ID NO: 49)
ATGTCCGTCCTGACGCCGCTGCTGCTGCGGGGCTTGACAGGCTCGGCCC
GGCGGCTCCCAGTGCCGCGCGCCAAGATCCATTCGTTG.

In some embodiments, the IMM-MLS can be huROMK MLS, which has the nucleic acid sequence:

(SEQ ID NO: 50)
ATGAATGCTTCCAGTCGGAATGTGTTTGACACGTTGATCAGGGTGTTGA
CAGAAAGTATGTTCAAACATCTTCGGAAATGGGTCGTCACTCGCTTTTT
TGGGCATTCTCGGCAAAGAGCAAGGCTAGTCTCCAAAGATGGAAGGTGC
AACATAGAATTTGGCAATGTGGAGGCACAGTCAAGGTTTATATTCTTTG
TGGACATCTGGACAACGGTACTTGACCTCAAGTGGAGATACAAAATGAC
CATTTTCATCACAGCCTTCTTGGGGAGTTGGTTTTTCTTTGGTCTCCTG
TGGTATGCAGTAGCGTACATTCACAAAGACCTCCCGGAATTCCATCCTT
CTGCCA.

Extracellular Vesicles

Also provided herein are extracellular vesicles (EVs) for delivery of the disclosed mitochondrial optogenetics-based gene therapies. Exemplary extracellular vesicles may include but are not limited to exosomes. However, the term “extracellular vesicles” should be interpreted to include all nanometer-scale lipid vesicles that are secreted by cells such as secreted vesicles formed from lysosomes.

EVs are cell-derived vesicles with a closed double-layer membrane structure. According to their size and density, EVs mainly include exosomes (30-150 nm), micro vesicles (MVs) (100-1000 nm), and apoptotic bodies or cancer related oncosomes (1-10 μm). EVs are able to carry various molecules, such as proteins, lipids and RNAs on their surface as well as within their lumen. The EV and exosomal surface proteins can mediate organ-specific homing of circulating EVs.

EVs are produced by many different types of cells including immune cells such as B lymphocytes, T lymphocytes, dendritic cells (DCs) and most cells. EVs are also produced, for example, by glioma cells, platelets, reticulocytes, neurons, intestinal epithelial cells and tumor cells. EVs for use in the disclosed compositions and methods can be derived from any suitable cells, including the cells identified above. EVs have also been isolated from physiological fluids, such as plasma, urine, amniotic fluid and malignant effusions. Non-limiting examples of suitable EVs producing cells for mass production include dendritic cells (e.g., immature dendritic cell), Human Embryonic Kidney 293 (HEK) cells, 293T cells, Chinese hamster ovary (CHO) cells, and human ESC-derived mesenchymal stem cells.

EVs can also be obtained from any autologous patient-derived, heterologous haplotype-matched or heterologous stem cells so to reduce or avoid the generation of an immune response in a patient to whom the EVs are delivered. Any EV-producing cell can be used for this purpose.

EVs produced from cells can be collected from the culture medium by any suitable method. Typically a preparation of EVs can be prepared from cell culture or tissue supernatant by centrifugation, filtration or combinations of these methods. For example, EVs can be prepared by differential centrifugation, that is low speed (<20000 g) centrifugation to pellet larger particles followed by high speed (>100000 g) centrifugation to pellet EVs, size filtration with appropriate filters (for example, 0.22 μ|η filter), gradient ultracentrifugation (for example, with sucrose gradient) or a combination of these methods.

In one embodiment, the EVs comprising the disclosed fusion protein are obtained by culturing a cell expressing the fusion protein and subsequently isolating indirectly modified EVs from the culture medium.

The disclosed EVs may be administered to a subject by any suitable means. Administration to a human or animal subject may be selected from parenteral, intramuscular, intracerebral, intravascular, subcutaneous, or transdermal administration. Typically the method of delivery is by injection. Preferably the injection is intramuscular or intravascular (e.g. intravenous). A physician will be able to determine the required route of administration for each particular patient.

The EVs are preferably delivered as a composition. The composition may be formulated for parenteral, intramuscular, intracerebral, intravascular (including intravenous), subcutaneous, or transdermal administration. Compositions for parenteral administration may include sterile aqueous solutions which may also contain buffers, diluents and other suitable additives. The EVs may be formulated in a pharmaceutical composition, which may include pharmaceutically acceptable carriers, thickeners, diluents, buffers, preservatives, and other pharmaceutically acceptable carriers or excipients and the like in addition to the EVs.

EVs may be administered within a pharmaceutically-acceptable diluent, carrier, or excipient, in unit dosage form. Conventional pharmaceutical practice may be employed to provide suitable formulations or compositions to administer the compounds to patients suffering from a disease (e.g., cancer). Administration may begin before the patient is symptomatic. Any appropriate route of administration may be employed, for example, administration may be parenteral, intravenous, intraarterial, subcutaneous, intratumoral, intramuscular, intracranial, intraorbital, ophthalmic, intraventricular, intrahepatic, intracapsular, intrathecal, intracisternal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols.

The disclosed extracellular vesicles further may comprise an agent, such as a therapeutic agent, where the extracellular vesicles deliver the agent to a target cell. Agents comprised by the extracellular vesicles may include but are not limited to therapeutic drugs (e.g., small molecule drugs), therapeutic proteins, and therapeutic nucleic acids (e.g., therapeutic RNA). In some embodiments, the disclosed extracellular vesicles comprise a therapeutic RNA as a so-called “cargo RNA.” For example, in some embodiments the fusion protein further may comprise an RNA-domain (e.g., at a cytosolic C-terminus of the fusion protein) that binds to one or more RNA-motifs present in the cargo RNA in order to package the cargo RNA into the extracellular vesicle, prior to the extracellular vesicles being secreted from a cell. As such, the fusion protein may function as both of a “targeting protein” and a “packaging protein.” In some embodiments, the packaging protein may be referred to as extracellular vesicle-loading protein or “EV-loading protein.” (See Hung and Leonard, “A platform for actively loading cargo RNA to elucidate limiting steps in EV-mediated delivery,” J. Extracellular Vesicles, 2016, 5: 31027, published 13 May 2016, the content of which is incorporated herein by reference in its entirety.)

Formulations

Also provided herein are formulations that can include an amount of fusion proteins or viral vectors described herein and a pharmaceutical carrier appropriate for administration to an individual in need thereof. The individual in need thereof can have or can be suspected of a cancer in need of treatment or prevention.

Formulations can be administered via any suitable administration route. For example, the disclosed fusion proteins or viral vectors can be formulated for parenteral delivery, such as injection or infusion, in the form of a solution or suspension. The formulation can be administered via any route, such as, the blood stream or directly to the organ or tissue to be treated.

Parenteral formulations can be prepared as aqueous compositions using techniques is known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the disclosed fusion proteins or viral vectors can be prepared in water or another solvent or dispersing medium suitably mixed with one or more pharmaceutically acceptable excipients including, but not limited to, surfactants, dispersants, emulsifiers, pH modifying agents, and combination thereof.

Suitable surfactants can be anionic, cationic, amphoteric or nonionic surface-active agents. Suitable anionic surfactants include, but are not limited to, those containing carboxylate, sulfonate and sulfate ions. Suitable anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Suitable cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Suitable nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation can also contain an antioxidant to prevent degradation of the disclosed DNA origami nanostructures.

The formulation can be buffered to a pH of 3-8 for parenteral administration upon reconstitution. Suitable buffers include, but are not limited to, phosphate buffers, acetate buffers, and citrate buffers.

Water-soluble polymers can be used in the formulations for parenteral administration. Suitable water-soluble polymers include, but are not limited to, polyvinylpyrrolidone, dextran, carboxymethylcellulose, and polyethylene glycol. Sterile injectable solutions can be prepared by incorporating the disclosed DNA origami nanostructures in the required amount in the appropriate solvent or dispersion medium with one or more of the excipients listed above, as required, followed by filtered sterilization. Dispersions can be prepared by incorporating the various sterilized disclosed DNA origami nanostructures into a sterile vehicle which contains the basic dispersion medium and the required other ingredients from those listed above. Sterile powders for the preparation of sterile injectable solutions can be prepared by vacuum-drying and freeze-drying techniques, which yields a powder of the disclosed DNA origami nanostructures plus any additional desired ingredient from a previously sterile-filtered solution thereof. The powders can be prepared in such a manner that the particles are porous in nature, which can increase dissolution of the particles. Methods for making porous particles are well known in the art.

Pharmaceutical formulations for parenteral administration can be in the form of a sterile aqueous solution or suspension of particles formed from one or more disclosed DNA origami nanostructures. Acceptable solvents include, for example, water, Ringer's solution, phosphate buffered saline (PBS), and isotonic sodium chloride solution. The formulation can also be a sterile solution, suspension, or emulsion in a nontoxic, parenterally acceptable diluent or solvent such as 1,3-butanediol.

In some instances, the formulation can be distributed or packaged in a liquid form. In other embodiments, formulations for parenteral administration can be packed as a solid, obtained, for example by lyophilization of a suitable liquid formulation. The solid can be reconstituted with an appropriate carrier or diluent prior to administration.

Solutions, suspensions, or emulsions for parenteral administration can be buffered with an effective amount of buffer necessary to maintain a pH suitable for ocular administration. Suitable buffers include, but are not limited to, acetate, borate, carbonate, citrate, and phosphate buffers.

Solutions, suspensions, or emulsions for parenteral administration can also contain one or more tonicity agents to adjust the isotonic range of the formulation. Suitable tonicity agents include, but are not limited to, glycerin, mannitol, sorbitol, sodium chloride, and other electrolytes.

Solutions, suspensions, or emulsions for parenteral administration can also contain one or more preservatives to prevent bacterial contamination of the ophthalmic preparations. Suitable preservatives include, but are not limited to, polyhexamethylenebiguanidine (PHMB), benzalkonium chloride (BAK), stabilized oxychloro complexes (otherwise known as Purite®), phenylmercuric acetate, chlorobutanol, sorbic acid, chlorhexidine, benzyl alcohol, parabens, thimerosal, and mixtures thereof.

Solutions, suspensions, or emulsions, use of nanotechnology including nanoformulations for parenteral administration can also contain one or more excipients, such as dispersing agents, wetting agents, and suspending agents.

Additional Active Agents

In some embodiments, an amount of one or more additional active agents are included in the pharmaceutical formulation containing fusion proteins or viral vectors. Suitable additional active agents include, but are not limited to, DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, and chemotherapeutics (anti-cancer drugs). Other suitable additional active agents include, sensitizers (such as radiosensitizers). The disclosed DNA origami nanostructures can be used as a monotherapy or in combination with other active agents for treatment or prevention of a disease or disorder.

Suitable hormones include, but are not limited to, amino-acid derived hormones (e.g. melatonin and thyroxine), small peptide hormones and protein hormones (e.g. thyrotropin-releasing hormone, vasopressin, insulin, growth hormone, luteinizing hormone, follicle-stimulating hormone, and thyroid-stimulating hormone), eiconsanoids (e.g. arachidonic acid, lipoxins, and prostaglandins), and steroid hormones (e.g. estradiol, testosterone, tetrahydro testosteron cortisol).

Suitable immunomodulators include, but are not limited to, prednisone, azathioprine, 6-MP, cyclosporine, tacrolimus, methotrexate, interleukins (e.g. IL-2, IL-7, and IL-12), cytokines (e.g. interferons (e.g. IFN-α, IFN-β, IFN-ε, IFN-κ, IFN-ω, and IFN-γ), granulocyte colony-stimulating factor, and imiquimod), chemokines (e.g. CCL3, CCL26 and CXCL7), cytosine phosphate-guanosine, oligodeoxynucleotides, glucans, antibodies, and aptamers).

Suitable antipyretics include, but are not limited to, non-steroidal anti-inflammants (e.g. ibuprofen, naproxen, ketoprofen, and nimesulide), aspirin and related salicylates (e.g. choline salicylate, magnesium salicylae, and sodium salicaylate), paracetamol/acetaminophen, metamizole, nabumetone, phenazone, and quinine.

Suitable anxiolytics include, but are not limited to, benzodiazepines (e.g. alprazolam, bromazepam, chlordiazepoxide, clonazepam, clorazepate, diazepam, flurazepam, lorazepam, oxazepam, temazepam, triazolam, and tofisopam), serotenergic antidepressants (e.g. selective serotonin reuptake inhibitors, tricyclic antidepresents, and monoamine oxidase inhibitors), mebicar, afobazole, selank, bromantane, emoxypine, azapirones, barbituates, hyxdroxyzine, pregabalin, validol, and beta blockers.

Suitable antipsychotics include, but are not limited to, benperidol, bromoperidol, droperidol, haloperidol, moperone, pipaperone, timiperone, fluspirilene, penfluridol, pimozide, acepromazine, chlorpromazine, cyamemazine, dizyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, tiotixene, zuclopenthixol, clotiapine, loxapine, prothipendyl, carpipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzaprine, paliperidone, perospirone, quetiapine, remoxipride, risperidone, sertindole, trimipramine, ziprasidone, zotepine, alstonie, befeprunox, bitopertin, brexpiprazole, cannabidiol, cariprazine, pimavanserin, pomaglumetad methionil, vabicaserin, xanomeline, and zicronapine.

Suitable analgesics include, but are not limited to, paracetamol/acetaminophen, non-steroidal anti-inflammants (e.g. ibuprofen, naproxen, ketoprofen, and nimesulide), COX-2 inhibitors (e.g. rofecoxib, celecoxib, and etoricoxib), opioids (e.g. morphine, codeine, oxycodone, hydrocodone, dihydromorphine, pethidine, buprenorphine), tramadol, norepinephrine, flupiretine, nefopam, orphenadrine, pregabalin, gabapentin, cyclobenzaprine, scopolamine, methadone, ketobemidone, piritramide, and aspirin and related salicylates (e.g. choline salicylate, magnesium salicylae, and sodium salicaylate).

Suitable antispasmodics include, but are not limited to, mebeverine, papverine, cyclobenzaprine, carisoprodol, orphenadrine, tizanidine, metaxalone, methodcarbamol, chlorzoxazone, baclofen, dantrolene, baclofen, tizanidine, and dantrolene.

Suitable anti-inflammatories include, but are not limited to, prednisone, non-steroidal anti-inflammants (e.g. ibuprofen, naproxen, ketoprofen, and nimesulide), COX-2 inhibitors (e.g. rofecoxib, celecoxib, and etoricoxib), and immune selective anti-inflammatory derivatives (e.g. submandibular gland peptide-T and its derivatives).

Suitable anti-histamines include, but are not limited to, H₁-receptor antagonists (e.g. acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbromapheniramine, dexchlorpheniramine, dimenhydrinate, dimetindene, diphenhydramine, doxylamine, ebasine, embramine, fexofenadine, hydroxyzine, levocetirzine, loratadine, meclozine, mirtazapine, olopatadine, orphenadrine, phenindamine, pheniramine, phenyltoloxamine, promethazine, pyrilamine, quetiapine, rupatadine, tripelennamine, and triprolidine), H₂-receptor antagonists (e.g. cimetidine, famotidine, lafutidine, nizatidine, rafitidine, and roxatidine), tritoqualine, catechin, cromoglicate, nedocromil, and β2-adrenergic agonists.

Suitable anti-infectives include, but are not limited to, amebicides (e.g. nitazoxanide, paromomycin, metronidazole, tnidazole, chloroquine, and iodoquinol), aminoglycosides (e.g. paromomycin, tobramycin, gentamicin, amikacin, kanamycin, and neomycin), anthelmintics (e.g. pyrantel, mebendazole, ivermectin, praziquantel, abendazole, miltefosine, thiabendazole, oxamniquine), antifungals (e.g. azole antifungals (e.g. itraconazole, fluconazole, posaconazole, ketoconazole, clotrimazole, miconazole, and voriconazole), echinocandins (e.g. caspofungin, anidulafungin, and micafungin), griseofulvin, terbinafine, flucytosine, and polyenes (e.g. nystatin, and amphotericin b), antimalarial agents (e.g. pyrimethamine/sulfadoxine, artemether/lumefantrine, atovaquone/proquanil, quinine, hydroxychloroquine, mefloquine, chloroquine, doxycycline, pyrimethamine, and halofantrine), antituberculosis agents (e.g. aminosalicylates (e.g. aminosalicylic acid), isoniazid/rifampin, isoniazid/pyrazinamide/rifampin, bedaquiline, isoniazid, ethanmbutol, rifampin, rifabutin, rifapentine, capreomycin, and cycloserine), antivirals (e.g. amantadine, rimantadine, abacavir/lamivudine, emtricitabine/tenofovir, cobicistat/elvitegravir/emtricitabine/tenofovir, efavirenz/emtricitabine/tenofovir, avacavir/lamivudine/zidovudine, lamivudine/zidovudine, emtricitabine/tenofovir, emtricitabine/opinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpiviirine, delaviridine, nevirapine, entecavir, lamivudine, adefovir, sofosbuvir, didanosine, tenofovir, avacivr, zidovudine, stavudine, emtricitabine, xalcitabine, telbivudine, simeprevir, boceprevir, telaprevir, lopinavir/ritonavir, fosamprenvir, dranuavir, ritonavir, tipranavir, atazanavir, nelfinavir, amprenavir, indinavir, sawuinavir, ribavirin, valcyclovir, acyclovir, famciclovir, ganciclovir, and valganciclovir), carbapenems (e.g. doripenem, meropenem, ertapenem, and cilastatin/imipenem), cephalosporins (e.g. cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), glycopeptide antibiotics (e.g. vancomycin, dalbavancin, oritavancin, and telvancin), glycylcyclines (e.g. tigecycline), leprostatics (e.g. clofazimine and thalidomide), lincomycin and derivatives thereof (e.g. clindamycin and lincomycin), macrolides and derivatives thereof (e.g. telithromycin, fidaxomicin, erthromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, beta lactam antibiotics (benzathine penicillin (benzatihine and benzylpenicillin), phenoxymethylpenicillin, cloxacillin, flucoxacillin, methicillin, temocillin, mecillinam, azlocillin, mezlocillin, piperacillin, amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxacillin, dicloxacillin, nafcillin, cefazolin, cephalexin, cephalosporin C, cephalothin, cefaclor, cefamandole, cefuroxime, cefotetan, cefoxitin, cefiximine, cefotaxime, cefpodoxime, ceftazidime, ceftriaxone, cefepime, cefpirome, ceftaroline, biapenem, doripenem, ertapenem, faropenem, imipenem, meropenem, panipenem, razupenem, tebipenem, thienamycin, azrewonam, tigemonam, nocardicin A, taboxinine, and beta-lactam), quinolones (e.g. lomefloxacin, norfloxacin, ofloxacin, qatifloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, and sparfloxacin), sulfonamides (e.g. sulfamethoxazole/trimethoprim, sulfasalazine, and sulfasoxazole), tetracyclines (e.g. doxycycline, demeclocycline, minocycline, doxycycline/salicyclic acid, doxycycline/omega-3 polyunsaturated fatty acids, and tetracycline), and urinary anti-infectives (e.g. nitrofurantoin, methenamine, fosfomycin, cinoxacin, nalidixic acid, trimethoprim, and methylene blue).

Suitable chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, cytoxan, cyclophosphamide, decitabine, dexamethasone, docetaxel, hydroxyurea, decarbazine, leuprolide, epirubicin, oxaliplatin, asparaginase, estramustine, cetuximab, vismodegib, aspargainase erwinia chyrsanthemi, amifostine, etoposide, flutamide, toremifene, fulvestrant, letrozole, degarelix, pralatrexate, methotrexate, floxuridine, obinutuzumab, gemcitabine, afatinib, imatinib mesylatem, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octretide, dasatinib, regorafenib, histrelin, sunitinib, siltuximab, omacetaxine, thioguanine (tioguanine), dabrafenib, erlotinib, bexarotene, temozolomide, thiotepa, thalidomide, BCG, temsirolimus, bendamustine hydrochloride, triptorelin, aresnic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, all-trans retinoic acid, and other anti-cancer agents listed elsewhere herein.

The disclosed gene therapy compositions may be an injectable preparation. Injectable preparations, for example, sterile injectable aqueous or oleaginous suspensions, may be formulated according to the known art using suitable dispersing or wetting agents and suspending agents. The sterile injectable preparation may also be a sterile injectable solution or suspension in a nontoxic parenterally acceptable diluent or solvent. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful. The disclosed gene therapy compositions may further comprise a pharmaceutically acceptable excipient.

Methods of Treating Cancer

The disclosed fusion proteins and viral vectors can be used in some cases to treat a subject with a cancer. The disclosed technique can effectively kill recurring, drug-resistant or heterogeneous cancers, and both low and high-grade cancers. In some embodiments, the cancer is a neuroendocrine tumor (NET) including pulmonary cancers, thyroid cancers and pancreatic cancers. In vitro studies showed that 60-90% of NET can be killed within one day post treatment of the disclosed gene therapy. In vivo studies showed that adenovirus drug stopped tumor growth and started shrinking tumor within 7 days post treatment, and lentivirus drug stopped or significantly (>60%) reduced tumor growth depending on drug dosage. In some embodiments, the cancer is a breast cancer, including HER2+, ER+, and triple negative breast cancers (TNBC). In vitro studies showed that 60-90% of breast cancer cells can be killed within one or two days post treatment. In some embodiments, the cancer is a glioblastoma multiforme (GBM), including wild type U251 and drug resistant U251-TMZ cells. In vitro studies showed that 60-90% of GBM can be killed within one or two days post treatment.

The cancer of the disclosed methods can in some embodiments be any cell in a subject undergoing unregulated growth, invasion, or metastasis. In some aspects, the cancer can be any neoplasm or tumor for which radiotherapy is currently used. Alternatively, the cancer can be a neoplasm or tumor that is not sufficiently sensitive to radiotherapy using standard methods. Thus, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T cell lymphoma, mycosis fungoides, Hodgkin's Disease, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

Administration of the disclosed compositions and formulations thereof can be systemic or localized. The compounds and formulations described herein can be administered to the subject in need thereof one or more times per day. In an embodiment, the compound(s) and/or formulation(s) thereof can be administered once daily. In some embodiments, the compound(s) and/or formulation(s) thereof can be administered given once daily. In another embodiment, the compound(s) and/or formulation(s) thereof can be administered is administered twice daily. In some embodiments, when administered, an effective amount of the compounds and/or formulations are administered to the subject in need thereof. The compound(s) and/or formulation(s) thereof can be administered one or more times per week. In some embodiments the compound(s) and/or formulation(s) thereof can be administered 1 day per week. In other embodiments, the compound(s) and/or formulation(s) thereof can be administered 2 to 7 days per week.

In some embodiments, the disclosed compositions and/or formulation(s) thereof, can be administered in a dosage form. The amount or effective amount of the compound(s) and/or formulation(s) thereof can be divided into multiple dosage forms. For example, the effective amount can be split into two dosage forms and the one dosage forms can be administered, for example, in the morning, and the second dosage form can be administered in the evening. Although the effective amount is given over two doses, in one day, the subject receives the effective amount. In some embodiments the effective amount is about 0.1 to about 1000 mg per day. The effective amount in a dosage form can range from about 0.1 mg/kg to about 1000 mg/kg. The dosage form can be formulated for oral, vaginal, intravenous, transdermal, subcutaneous, intraperitoneal, or intramuscular administration. Preparation of dosage forms for various administration routes are described elsewhere herein.

Indicators of toxicity are known in the art. For example, toxicity may cause damage to DNA, RNA, lipids, proteins, and various cellular compartments. Sub lethal doses of cytotoxic compositions may result in decreased cell proliferation. Lethal doses of toxic compositions may result in loss of membrane integrity and cell death. Cytotoxicity may be measured by standard cytotoxicity assays, such as assays that measure cell viability and cell death. Examples of standard cytotoxicity assays include MTT assays, ATP assays, Neutral Red uptake assays, ELISA, MTS assays, SRB assays, WST assays, and clonogenic assays. Toxicity may also be determined by measuring cell death, such as by apoptosis or necrosis.

The gene therapy composition may be administered to the subject by several different means. For instance, the composition may generally be administered parenterally, intraperitoneally, intravascularly, or intrapulmonarily. The composition may be administered to the subject in dosage unit formulations containing conventional nontoxic pharmaceutically acceptable carriers, adjuvants, and vehicles as desired. The composition may be administered parenterally.

The term parenteral as used herein includes subcutaneous, intravenous, intramuscular, intrathecal, or intrasternal injection, or infusion techniques. Formulation of pharmaceutical compositions is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Delivery methods are preferably those that are effective to circumvent the blood-brain barrier and are effective to deliver compositions to the central nervous system. For example, delivery methods may include the use of nanoparticles. Positively charged lipids are particularly preferred for such nanoparticles, and may include liposomes, niosomes, micelles, multilamellar vesicles, unilamellar vesicles, and polymersomes. The preparation of such lipid particles is well known in the art. See, e.g., U.S. Pat. No. 4,880,635 to Janoff et al.; U.S. Pat. No. 4,906,477 to Kurono et al.: U.S. Pat. No. 4,911,928 to Wallach; U.S. Pat. No. 4,917,951 to Wallach; U.S. Pat. No. 4,920,016 to Allen et al.: U.S. Pat. No. 4,921,757 to Wheatley et al.; etc.

In some embodiments, the disclosed gene therapy composition may be administered to the subject in a bolus once, or multiple times. When administered multiple times, the composition may be administered at regular intervals or at intervals that may vary during the treatment of a subject.

The composition may be administered by continuous infusion. Non-limiting examples of methods that may be used to deliver the compositions provided herein by continuous infusion may include pumps, wafers, gels, foams and fibrin clots. The composition may be delivered by continuous infusion using an osmotic pump. An osmotic mini pump contains a high-osmolality chamber that surrounds a flexible, yet impermeable, reservoir filled with the targeted delivery composition-containing vehicle. Subsequent to the subcutaneous implantation of this minipump, extracellular fluid enters through an outer semi-permeable membrane into the high-osmolality chamber, thereby compressing the reservoir to release the targeted delivery composition at a controlled, pre-determined rate. The targeted delivery composition, released from the pump, may be directed via a catheter to a stereotaxically placed cannula for infusion.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Characterize Mitochondrial Optogenetic-Induced Cytotoxicity in Heterogeneous Human Glioma Xenograft

Mitochondrial energetic heterogeneity, specifically tighter coupling between mitochondrial respiration and ATP synthesis, may underlie drug resistance in glioma cells (Griguer C E, et al. PLoS One. 2013 8(4):e61035; Oliva C R, et al. Oncotarget. 2015 6(6):4330-44; Oliva C R, et al. PLoS One. 2011 6(9):e24665; Oliva C R, et al. J Biol Chem. 2010 285(51):39759-67). A population of glioma stem cells (GSCs) responsible for chemoresistance relies on mitochondria (Oliva C R, et al. Oncotarget. 2015 6(6):4330-44). Finally, mitochondrial-targeting optogenetics approach can induce light-controlled mitochondrial uncoupling, Δψₘ depolarization and cell death in HeLa cells and U251 glioma cells. Thus, light-controlled direct disruption of IMM integrity may overcome the established mechanism of heterogeneity and chemoresistance in glioma, as the mechanism by which it is induced is at the core of all biological functions and independent of endogenous proteins.

Due to its high spatiotemporal resolution, ChR-based optogenetic technique has evolved as a powerful tool in basic and translational research to monitor and manipulate the plasma membrane excitability of a variety types of cells. The heterologous ChR2 protein can also be expressed in the inner mitochondrial membrane (IMM) of mammalian cells such as HeLa and H9C2 (Ernst P X, et al. AHA Scientific Sessions; Anaheim, Calif.2017; Tkatch T, et al. Proc Natl Acad Sci USA. 2017 114(26):E5167-E76). Mitochondrial ChR2-expressing cells subjected to sustained light illumination displayed depolarized ΔΨₘ that led to cell death through intrinsic apoptosis mechanism.

In this example, ChR2 protein is expressed in the IMM of TMZ-sensitive and resistant patient derived xenograft (PDX) as well as glioma stem cells (GSC) known to be intrinsically chemoresistant to achieve targeting induction of cell death by visible light.

Examine Mitochondrial Optogenetic-Mediated Cell Death in PDX GBM Cells.

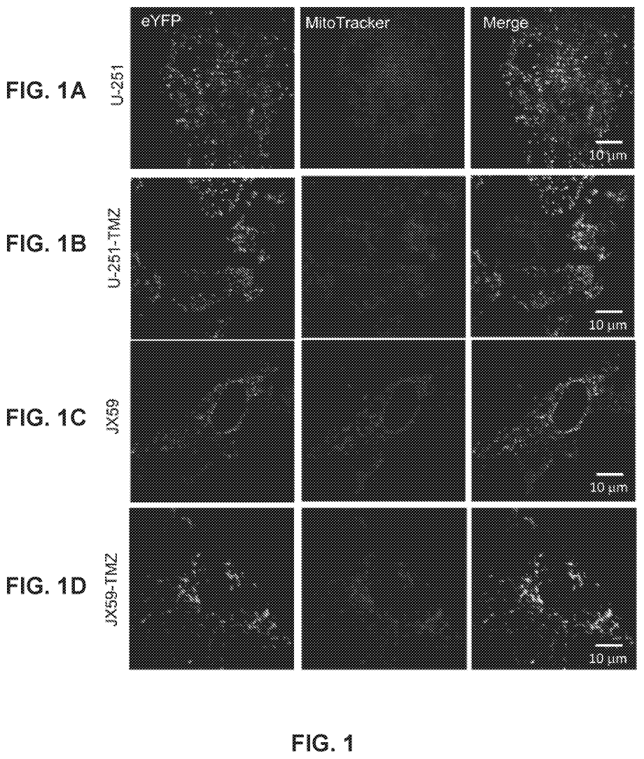

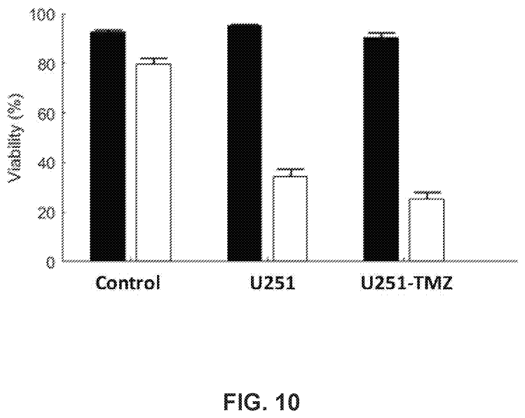

Targeting mitochondrial expression. A mitochondrial targeting sequence (ABCB) has been identified that effectively imported ChR2 protein to H9C2 and HeLa mitochondria (Ernst P, et al. AHA Scientific Sessions; Anaheim, Calif.2017). An adenovirus encoding this gene was created (ABCB-ChR2) and used to infect chemosensitive U251 and chemoresistant U-TMZ glioma cells. The resulting ChR2 protein expression and mitochondrial localization in both cell lines was confirmed by colocalization of eYFP (fused with ChR2) and MitoTracker (a mitochondrial dye) (FIGS. 1A and 1B). This adenovirus could also induce ChR2 expression in mitochondria of GBM PDX cells (TMZ-sensitive JX59 and TMZ-resistant JX59T, FIGS. 10 and 1D). As control, separate groups of PDX lines are infected by adenovirus encoding ABCB-YFP gene. Expression and intracellular localization of ABCB-YFP or ABCB-ChR2 is determined using confocal microscopy, immunostaining, and proteinase K protection assay.

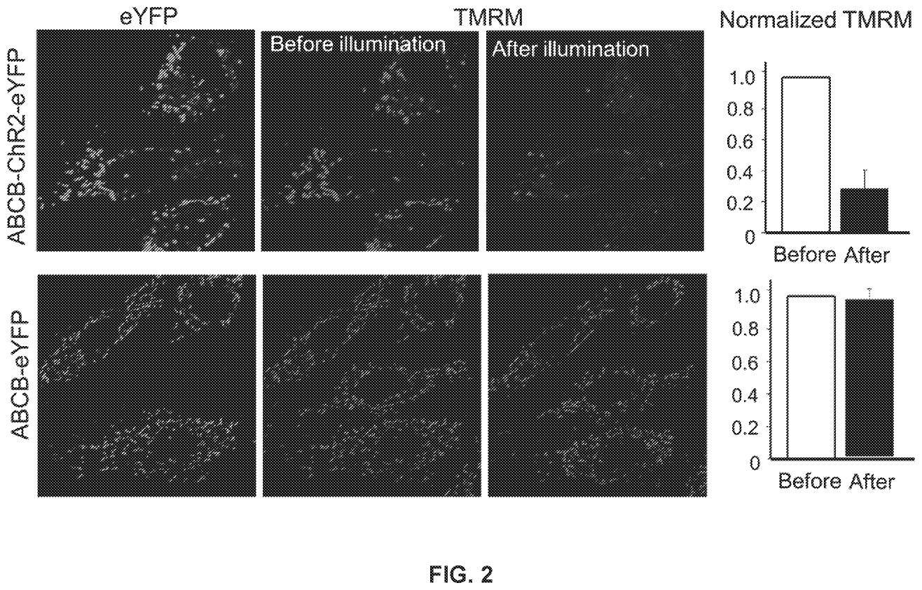

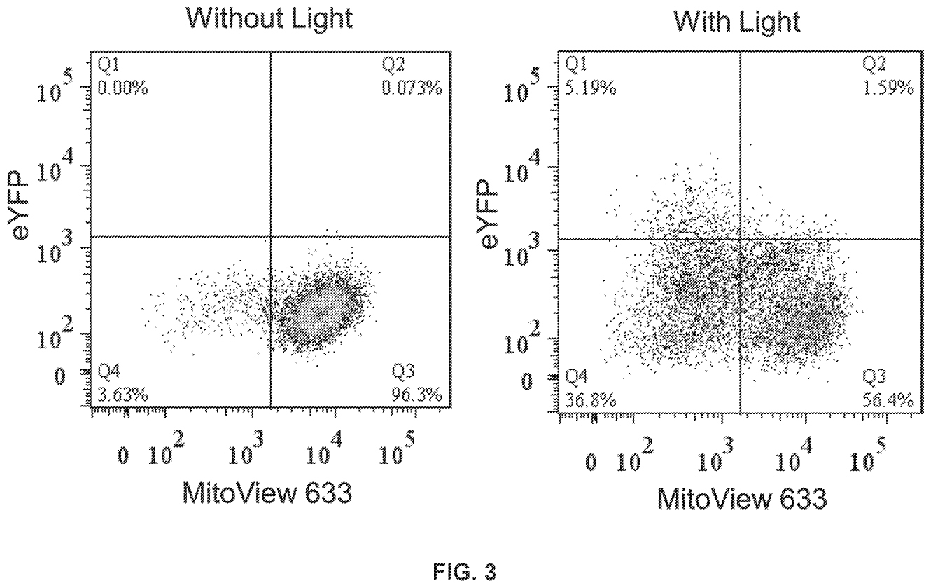

Light induced mitochondrial depolarization. Impulse blue light illumination led to mitochondrial depolarization in ABCB-ChR2-expressing, but not ABCB-YFP-expressing, H9C2 myoblast cells (FIG. 2), indicating that the Δψₘ depolarization is caused by the light-activated IMM ChR2 currents. Notably, blue light illumination (12 hours) also caused Δψₘ depolarization in chemoresistant JX59T cells expressing ABCB-ChR2 (FIG. 3). To determine the light-dependence and time course of optogenetics-mediated Δψₘ depolarization, GBM PDX cells expressing ABCB-ChR2 are illuminated with blue LED light of various intensities (from 0.1 to 20 mW/mm²) (Boyden E S, et al. Nat Neurosci. 2005 8(9):1263-8; Cardin J A, et al. Nat Protoc. 2010 5(2):247-54; Jia Z, et al. Circulation Arrhythmia and electrophysiology. 2011 4(5):753-60; Wang H, et al. Proc Natl Acad Sci USA. 2007 104(19):8143-8). Δψₘ at the baseline, 4, 8, 12, and 24 hours of illumination is analyzed by FACS and confocal microscopy. Results are compared between TMZ-sensitive and TMZ-resistant glioma PDX cells to determine: 1) effect of light intensity on Δψₘ depolarization, and 2) whether light illumination differentially influences Δψₘ of TMZ-sensitive and resistant glioma cells with different genetic backgrounds. The PDX cells expressing ABCB-YFP are used as negative controls.

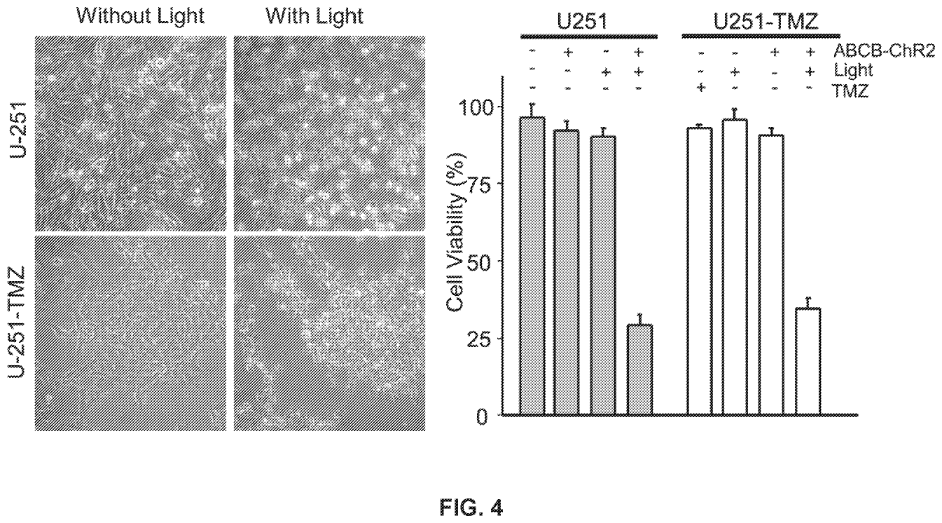

Mitochondrial optogenetic-induced glioma cell death. Sustained blue light illumination (24 hours) caused significantly reduced proliferation and viability of U251 and U-TMZ cells (FIG. 4). Experiments are also conducted to examine whether mitochondrial optogenetics-mediated direct Δψₘ depolarization induces cell death in both the TMZ-sensitive and TMZ-resistant GBM PDX cells. Two days after adenovirus infection, ABCB-ChR2-expressing GBM PDX cells are illuminated by 475 nm LED light. Cells are collected at 4, 8, 12 and 24 hours of light illumination for cell death assay using trypan blue cell counting method. Cytotoxicity is quantified using LDH assay, AlamarBlue assay, and TUNEL assay. GBM PDX cells expressing ABCB-eYFP, astrocytes expressing ABCB-ChR2, and mitochondrial DNA depleted glioma cells (ρ°) (Oliva C R, et al. PLoS One. 2011 6(9):e24665) are subjected to the same light illumination and cytotoxicity assay as negative controls.

### Example 2: CoChR-Based Mitochondrial Optogenetics Induces Cell Death in Tumor Cells

Plasmid Construction

The ABCB fragment was PCR-amplified using blunt-end primers (Forward: 5′-ACTCACTATAGGGAGACCCAAGCTTGCCACCATGCGCGCCCCTT-3′ (SEQ ID NO:51), Reverse: 5′-TTCCCAGCATAACTGCAGCTGACAGTCTCCCG-3′ (SEQ ID NO:52)) from DNA template (pCAG-ABCB-ChR2-YFP-ER). The CoChR fragment was PCR-amplified using blunt-end primers (Forward: 5′-AGCTGCAGTTATGCTGGGAAACGGCAGC-3′ (SEQ ID NO:53), Reverse: 5′-TATCCTCCTCGCCCTTGCTCACGGATCCTGCTACTACCGGTGCCGC-3′ (SEQ ID NO:54)) from DNA template (pAAV-Syn-CoChR-GFP, Addgene #59070). These gene fragments were cloned in a pcDNA3.0 vector using Gibson Assembly (New England Biolabs) to produce the plasmid pcDNA3.0-ABCB-CoChR-mCherry.

CoChR Expression in Mitochondria

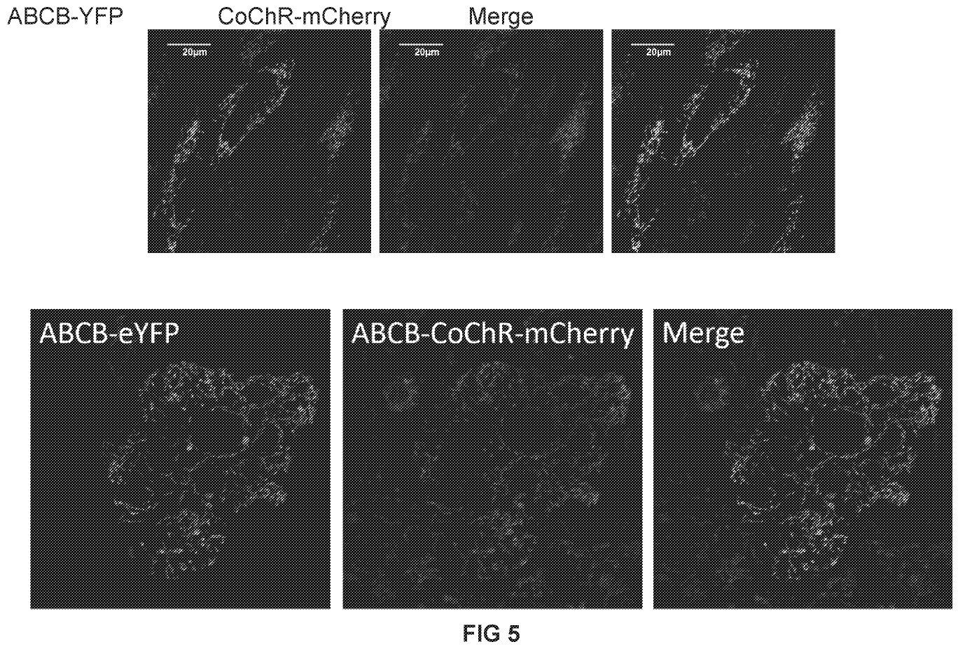

HeLa cells or BON tumor cells were co-transfected with DNA encoding mitochondrial-targeted CoChR-mCherry and mitochondrial-targeted eYFP using Lipofectamine 3000. Cells were then imaged two days later using an Olympus IX81 confocal microscope and FV-1000 laser scan head. FIG. 5 shows CoChR expression in mitochondria of HeLa (top) and BON (lower) tumor cells.

Light-Induced Cell Death in ABCB-CoChR Expressing Cells

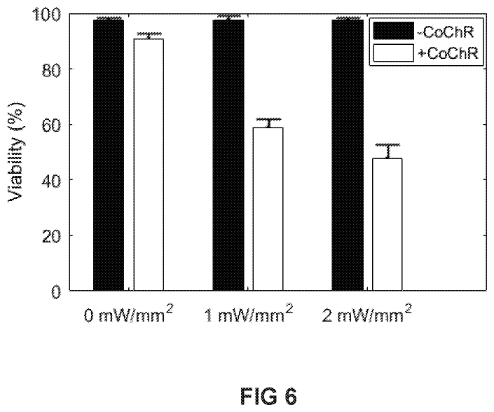

Tumor cells were seeded in a 96-well plate and transfected one day later using Lipofectamine 3000. Two days later cells were treated with pulsed LED light (4 Hz, 90 ms pulses) for either 8 hours (left) or 24 hours (right) at varying light intensities. Cells were then trypsinized and cell death was assayed using Trypan Blue. FIG. 6 shows light-induced cell death in ABCB-CoChR expressing tumor cells.

### Example 3: External Light-Independent Synthetic Optogenetics-Based Gene Therapy—Anti-Cancer Evaluation In Vitro and In Vivo

Adenovirus Production

Construction of Adenoviral Expression Vector

Gene of interest (GOI) was PCR amplified with blunt-end using primers (Forward: 5′-CACC GAATTC ACATTGATTATTGAG-3′ (SEQ ID NO:55), Reverse: 5′-TTTTTATTTCTAGACTACACCTCGTTCTCGTAGCAGAAC-3′ (SEQ ID NO:56)) and DNA template (CMV-ABCB140-ChR2-YFP-ER-Native). The GOI was cloned into the pENTR TOPO vector (Life Tech) to generate an entry vector and then transformed into One Shot Competent E. coli cells. The entry vector was sequenced using primers (M13 Forward (−20) (5′-GTAAAACGACGGCCAG-3′ (SEQ ID NO:57)) and M13 Reverse (5′-CAGGAAACAGCTATGAC-3′ (SEQ ID NO:58)).

Adenoviral expression vector was constructed by performing an LR recombination reaction via LR Clonase II enzyme and Proteinase K solution between the entry vector and destination vector pAd/PL-DEST (34.9 kb), followed by transformation using a recA, endA E. coli strain like TOP10, DH5αTM-T1R, or equivalent. The vector was sequenced using primers: pAd forward priming site (5′-GACTTTGACCGTTTACGTGGAGAC-3′ (SEQ ID NO:59)) and pAd reverse priming site (5′-CCTTAAGCCACGCCCACACATTTC-3′ (SEQ ID NO:60)).

Transfection of 293A Cells for Adenoviral Expression

293A cells, which contain a stably integrated copy of E1, were cultured in DMEM (high glucose) supplemented with 10% FBS, 0.1 mM MEM Non-Essential Amino Acids (NEAA), 2 mM L-glutamine, and 1% Pen-Strep (optional). The 293A cells were plated into well-plates or T-flasks and transfected with adenoviral expression vector using Lipofectamine 2000. Post transfection, culture medium was replaced with fresh, complete culture medium every 2-3 days until visible regions of cytopathic effect (CPE) are observed, and then the culture medium was replenish to allow infections to proceed until approximately 80% CPE is observed. Adenovirus-containing cells were harvested by squirting cells off the plate with a tissue culture pipette and lysed via three freeze/thaw cycles to release crude viral particle.

Amplification for Higher Titer Adenovirus Stock

The crude adenoviral stock was used to infect 293A cells at a multiplicity of infection (MOI) of 3 or 5. High titer adenovirus were harvested when the cells had rounded up and were floating or lightly attached to the tissue culture dish. The amplification process is very fast, only took 2-3 days, and scalable to any size tissue culture dish or plates.

Titration of Adenovirus Via Plaque Assay

293A cells were seeded into well plate one day before infection, viral stocks with 10 fold serial dilutions ranging from 10-4 to 10-9 were added into each well and agarose overlay solution was applied post-infection 1 day post infection as well as 2 days post-infection. Plaques were visible 8-12 days post-infection, which were stained using the vital dye, 3-[4, 5-dimethylthiazol-2-yl]-2, 5-diphenyltetrazolium bromide; thiazolyl blue (MTT) and counted in order to determine the titer of adenoviral stock.

Lentivirus Production

Construction of Expression Vector

The SMPU vector backbone for lentiviral construction was obtained via the double digestion of a plasmid (aMHC-puro rex-neo was a gift from Mark Mercola (Addgene plasmid #21230)) using BamHI/AgeI restriction enzymes. The open reading frame including luciferase coding sequence (hGluc or hRluc), channelrhodopsin gene, as well as promoter, OMA25, leading sequence, eYFP coding sequence, ER coding sequence, were all PCR amplified from in-house plasmids and cloned into the SMPU vector via Gibson assembly. The lentiviral vectors were amplified in NEB stable competent E. coli (High Efficiency) cells. All constructs have been confirmed by DNA sequencing.

Lentivirus Production

Lentiviral transgene plasmid was constructed as described above, while the lentiviral packaging plasmid psPAX2 (a gift from Didier Trono, Addgene plasmid #12260) and the envelop plasmid pMD2G (pVSV-G, a gift from Didier Trono, Addgene plasmid #12259) were used for lentiviral packaging. To produce lentivirus, 293T cells (ATCC) were cultured in a complete medium containing Dulbecco's Modified Eagle Medium (DMEM) supplemented with 10% FBS and 4 mM L-glutamine, and 8×10⁶ 293 T cells were plated into each 150-cm² dish and incubated in CO₂ one day prior to transfection. For transient transfection, 21 μg transfer plasmid, 14 μg packing plasmid and 7 μg envelop plasmid were mixed and added into ddH₂O, followed by the addition of 370 μL 2M CaCl₂ to obtain a final concentration of 0.25 M CaCl₂, and the total volume of DNA/CaCl₂ mixture was 3.0 mL for one 150-cm2 dish. The 2×HBS (hepes buffered saline, pH 7.0) solution was added dropwise into the DNA/CaCl₂ mixture while vortexing lightly. The mixture was incubated at room temperature for 20 min and added to the cells drop by drop. After the dishes with the transfected cells were incubated overnight, the transfection medium was replaced with fresh complete medium.

Viral supernatant were harvested 48 hour, 72 hour and 96 hour post-transfection. The supernatants were centrifuged at 500 g for 10 min to remove cells and large debris and filtered through 0.45 μm or 0.22 μm prior to concentration. Virus concentration was done by precipitation of lentivirus using PEG 6000, in which viral supernatant was mixed with 50% PEG 6000 solution and 4 M NaCl stock solution to obtain a final concentration of 8.5% for PEG 6000 and a final concentration of 0.4 M for NaCl and incubated at 4° C. for 1.5 hours. After the centrifugation at 7000 g for 10 min, white pellets were observed and re-suspended by 1×PBS buffer. The viral suspension was snap-frozen in crushed dry ice or liquid nitrogen and stored at −80° C.

Physical titers were determined by a p24 ELISA kit and for cell based assay. And biological titers were measured by infecting Hela cells with serial dilutions of concentrated lentivirus and analyzing the eYFP expression of infected cells with flow cytometry 48 hour post infection.

Results

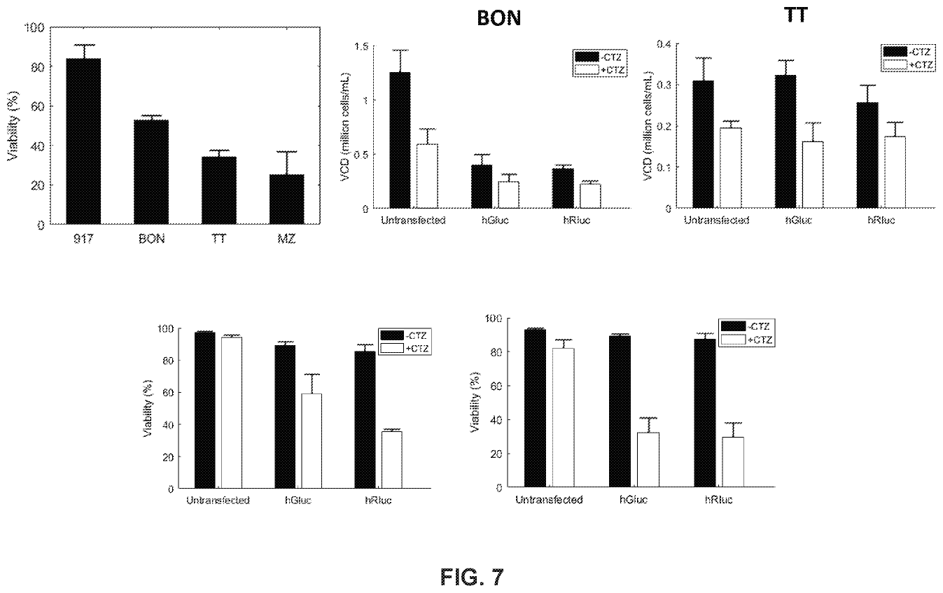

FIG. 7 shows luminoptogenetic cytotoxicity of three neuroendocrine tumor (NET) cell lines (BON, TT, MZ) as well as one negative (non-cancer) cell line (917). Cells given CTZ were treated for 48 hours with 60 μM EnduRen Live Cell Substrate. No external light is used. Significant cell death was seen in all three NET cell lines, with the highest cell death seen in the TT and MZ cells (Thyroid NET cell lines).

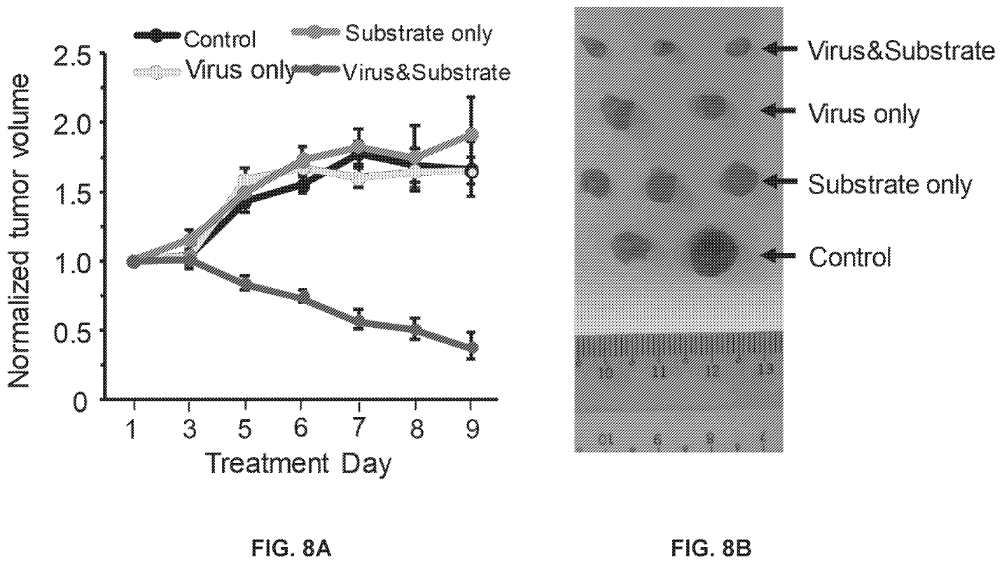

FIG. 8 shows results of a NET animal study. Growth curves for each mouse were calculated from tumor volumes before and after treatment, assuming no difference in growth rate over the entire period. 7-day treatment of NET tumor using adenovirus and lentivirus w/2× substrate completely stopped tumor growth. Dose of virus and substrate changed anti-NET efficacy significantly.

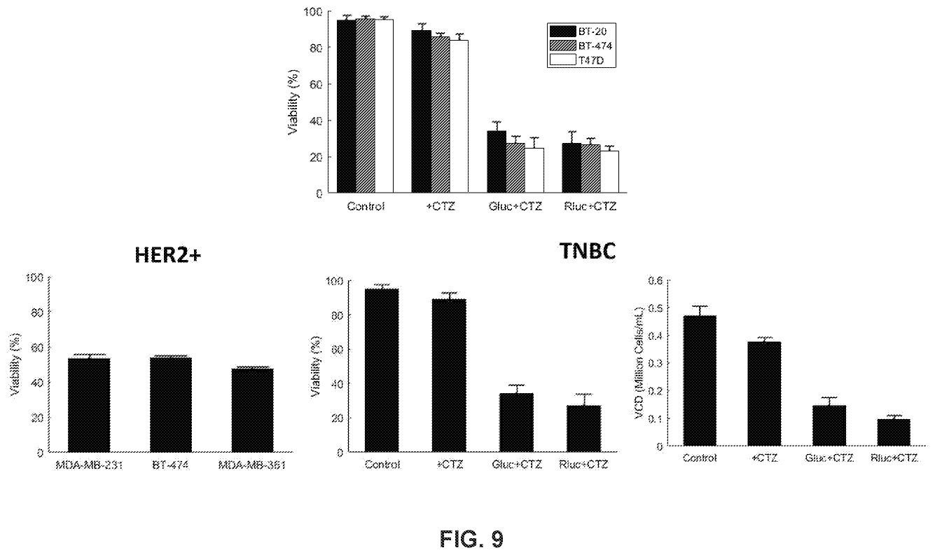

Breast cancer cells given substrate CTZ treated for 48 hours with 60 μM EnduRen Live Cell Substrate. Significant cell death was seen using both luciferase variants, with slightly higher cell death in cells expressing Renilla Luciferase (FIG. 9). Untransfected cells treated with just CTZ (control) showed minor cytotoxic effect and slightly inhibited cell growth. Breast cancer subtypes include HER2+(MDA-MB-231, BT-474, MDA-MB-361), ER+(T47D), and Triple negative breast cancer (TNBC) (BT-20 and others). The 3 HER2+cell lines are using ChR2 and light stimulation.

Anti-glioblastoma multiform (GBM) U251 and U251 resistant to chemotherapy Temozolomide (TMZ) were able to be successfully killed by this gene therapy (FIG. 10).

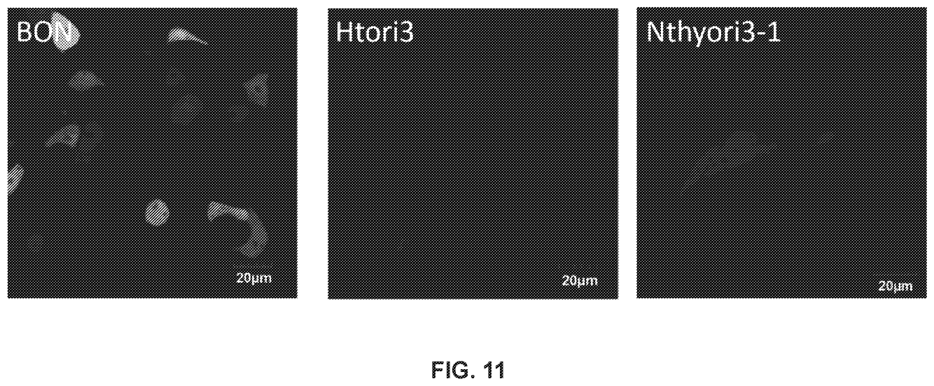

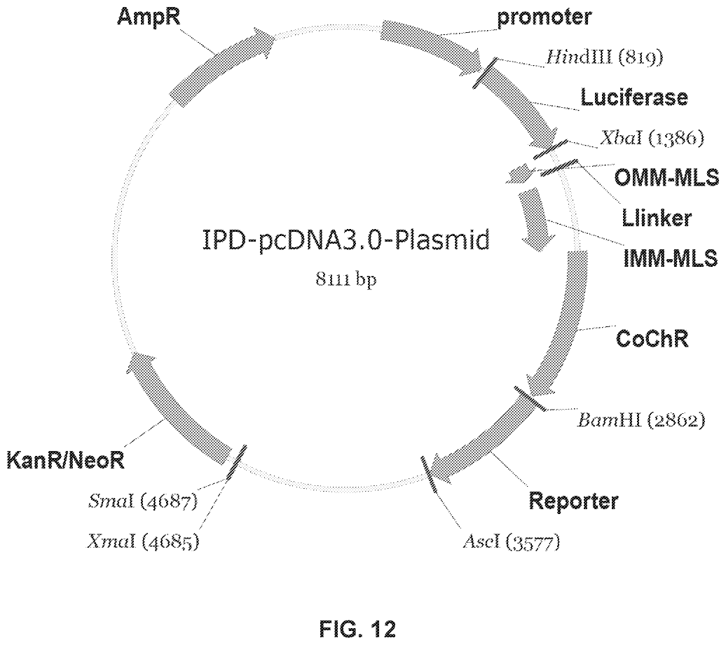

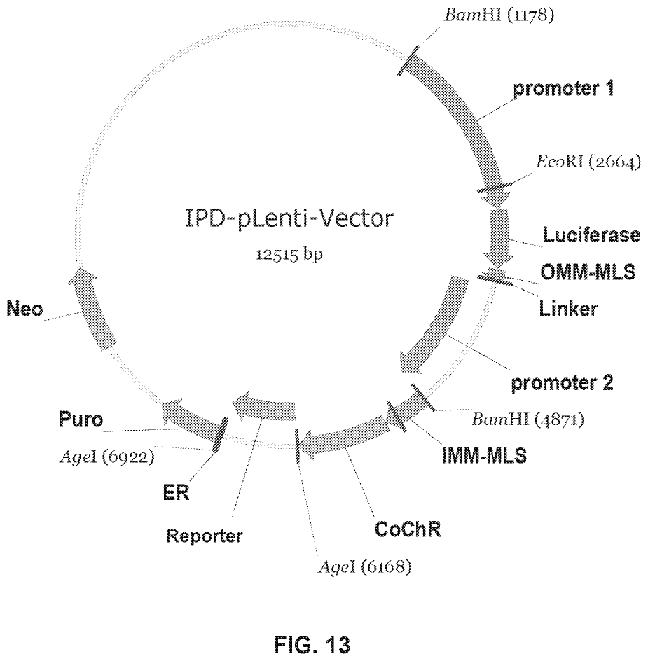

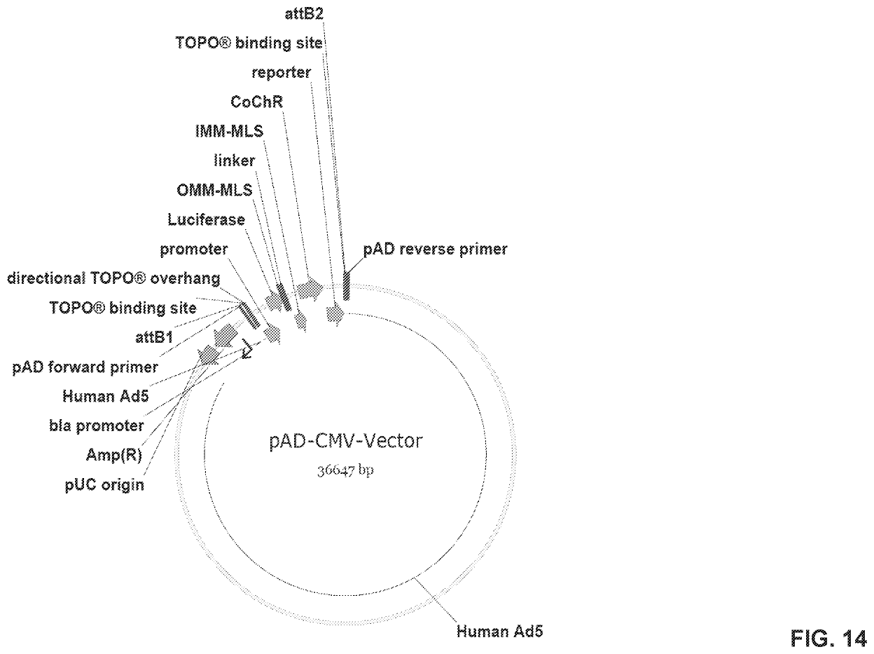

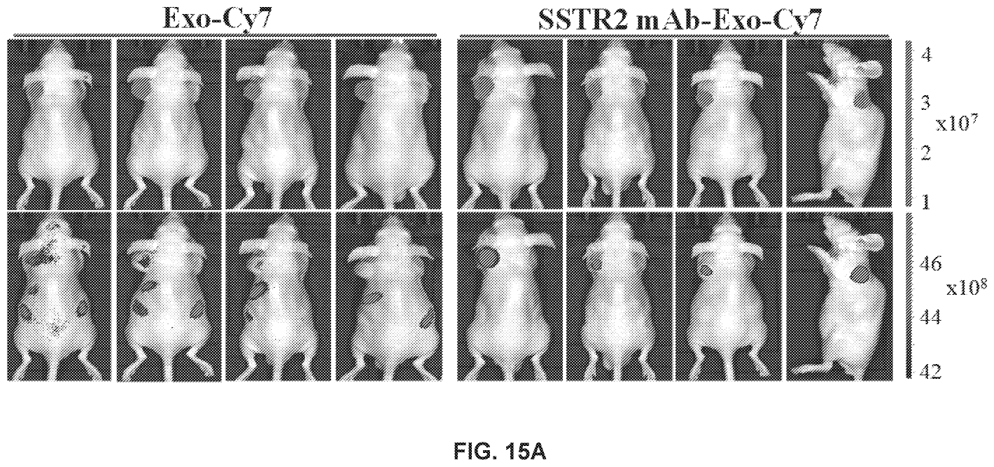

Cancer specific promoters were evaluated by confocal microscopy. cfos promoter resulted high gene expression cancer cell (BON, NET), but not or very low gene expression in normal cell (Htori3 and Nthyori3-1 (FIG. 11).

The expression and localization of CoChR was also evaluated by confocal microscopy. The CoChR channel was highly and properly expressed in the inner membrane of mitochondria of cancer cell (BON, NET) (FIG. 12).

### Example 4: Targeted Gene Delivery-mAb-Exo-AAV

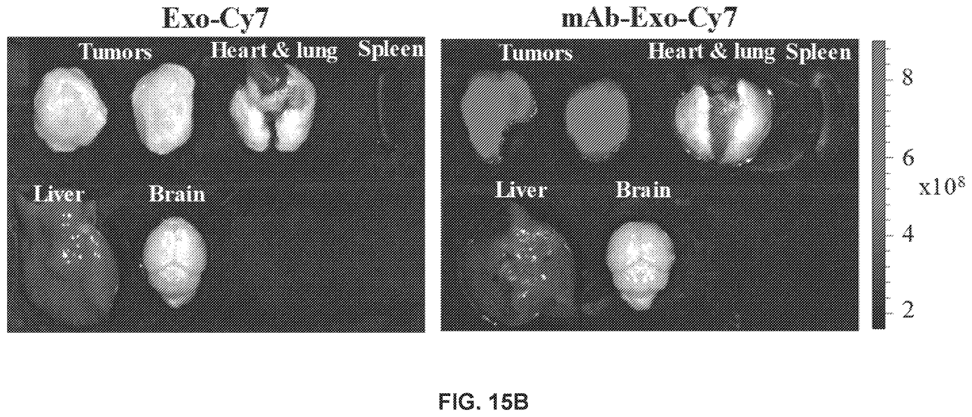

An innovative platform to produce Exo-AAV in scalable stirred-tank bioreactor was developed. First, HEK293A-AAV cells were adapted to serum-free suspension culture and cultivated in 1L-7.5L bioreactors with precise process control of pH 7.2, Temp 37° C., Agt 75 rpm, DO 50% and gas sparging VVM 0.01. Second, when VCD reached 0.75-1.5×10⁶ cells/mL, the HEK293A-AAV cells were transfected with large-scale pAAV-DJ/8 plasmid carrying RLuc-2A-ABCB-CoChR gene, helper plasmid and Rep-Cap plasmid (ratio of 1:1:1) supplemented with Cell Boost and GlutaMAX. Third, Exo-AAV were purified using size exclusion membrane and concentrator. The Nanosight analysis showed that the mean particle size of Exo-AAV was 139.2±3.4 nm and the yield was very high (>5×10¹³ Exo-AAV particles/L). Finally, the Exo-AAV was surface-tagged with anti-SSTR2 mAb (or any other mAb that can target cancer surface receptor) via DSPE-PEG-NHS linker to generate mAb-Exo-AAV, which were confirmed with Western blotting, transmission electron microscope (TEM) image, flow cytometry and in vivo cancer specific targeting animal study (FIGS. 15A and 15B).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A fusion protein, comprising a Chloromonas oogama channelrhodopsin (CoChR) photoreceptor linked to an inner mitochondrial membrane-mitochondrial localization signal (IMM-MLS).

2. The fusion protein of claim 1, wherein the IMM-MLS comprises a leading sequence from a mitochondrial inner membrane protein.

3. The fusion protein of claim 2, wherein the mitochondrial inner membrane protein is selected from ABCB10, ABCB140, Cytochrome C, and renal outer medullary potassium channel (ROMK).

4. The fusion protein of claim 1, wherein the CoChR comprises an amino acid having at least 90% sequence identity to SEQ ID NO:1.

5. An expression vector, comprising a nucleic acid sequence encoding a channelrhodopsin fusion protein operably linked to an expression control sequence and a nucleic acid sequence encoding a luciferase fusion protein operably linked to an expression control sequence, wherein the channelrhodopsin fusion protein comprises a channelrhodopsin linked to an inner mitochondrial membrane-mitochondrial localization signal (IMM-MLS), and wherein the luciferase fusion protein comprises a luciferase protein linked to an outer mitochondrial membrane-mitochondrial localization signal (OMM-MLS).

6. The expression vector of claim 5, wherein the channelrhodopsin is selected from Chloromonas oogama channelrhodopsin (CoChR), ChR2 (h134R), CHIEF, ChrimsonR, Chronos, CsChR, hChR2(C128A), hChR2(C128S), VChR1, and C1V1.

7. The expression vector of claim 5, wherein the IMM-MLS comprises a leading sequence from a mitochondrial inner membrane protein selected from ABCB10, ABCB140, Cytochrome C, and renal outer medullary potassium channel (ROMK).

8. The expression vector of claim 5, wherein the luciferase is selected from hRluc, hGluc, Nluc, M23hGluc and sbGluc.

9. The expression vector of claim 5, wherein the OMM-MLS comprises OMA25 or TOM20.

10. The expression vector of claim 5, wherein the nucleic acid sequence encoding a channelrhodopsin fusion protein and the nucleic acid sequence encoding a luciferase fusion protein are operably linked to the same expression control sequence and are separated by a self-cleavable linker or internal ribosome entry site (IRES).

11. The expression vector of claim 5, wherein the expression control sequence comprises a tissue specific promoter or cancer-specific promoter.

12. The expression vector of claim 5, further comprising a nucleic acid encoding a fluorophore.

13. The expression vector of claim 5, wherein the vector comprises a viral vector.

14. The expression vector of claim 13, wherein the viral vector is an adeno-associated virus (AAV) vector.

15. An extracellular vesicle comprising the expression vector of claim 5.

16. The extracellular vesicle of claim 15, comprising an antibody displayed on its surface, wherein the antibody is specific for a tumor antigen.

17. The extracellular vesicle of claim 16, wherein the tumor antigen comprises SSTR2.

18. The extracellular vesicle of claim 15, wherein the extracellular vesicle is an exosome.

19. A method for treating cancer in a subject, comprising administering to the subject the expression vector of claim 5.

20. The method of claim 19, wherein the cancer comprises a glioblastoma multiforme (GBM), a breast cancer, or a neuroendocrine tumor.


SEQUENCE LISTING (60 sequences)

SEQ ID NO: 1 - protein, 288 aa, Artificial Sequence
  Synthetic Construct
MLGNGSAIVPIDQCFCLAWTDSLGSDTEQLVANILQWFAFGFSILILMFYAYQTWRATCGWEEVYVCCVELTKVIIEFFHEFDDPSMLYLANGHRVQWLRYAEWLLTCPVILIHLSNLTGLKDDYSKRTMRLLVSDVGTIVWGATSAMSTGYVKVIFFVLGCIYGANTFFHAAKVYIESYHVVPKGRPRTVVRIMAWLFFLSWGMFPVLFVVGPEGFDAISVYGSTIGHTIIDLMSKNCWGLLGHYLRVLIHQHIIIYGDIRKKTKINVAGEEMEVETMVDQEDEETV

SEQ ID NO: 2 - DNA, 864 bp, Artificial Sequence
  Synthetic Construct
atgctgggaaacggcagcgccattgtgcctatcgaccagtgcttttgcctggcttggaccgacagcctgggaagcgatacagagcagctggtggccaacatcctccagtggttcgccttcggcttcagcatcctgatcctgatgttctacgcctaccagacttggagagccacttgcggttgggaggaggtctacgtctgttgcgtcgagctgaccaaggtcatcatcgagttcttccacgagttcgacgaccccagcatgctgtacctggctaacggacaccgagtccagtggctgagatacgcagagtggctgctgacttgtcccgtcatcctgatccacctgagcaacctgaccggcctgaaggacgactacagcaagcggaccatgaggctgctggtgtcagacgtgggaaccatcgtgtggggagctacaagcgccatgagcacaggctacgtcaaggtcatcttcttcgtgctgggttgcatctacggcgccaacaccttcttccacgccgccaaggtgtatatcgagagctaccacgtggtgccaaagggcagacctagaaccgtcgtgcggatcatggcttggctgttcttcctgtcttggggcatgttccccgtgctgttcgtcgtgggaccagaaggattcgacgccatcagcgtgtacggctctaccattggccacaccatcatcgacctcatgagcaagaattgttggggcctgctgggacactatctgagagtgctgatccaccagcacatcatcatctacggcgacatccggaagaagaccaagatcaacgtggccggcgaggagatggaagtggagaccatggtggaccaggaggacgaggagacagtg

SEQ ID NO: 3 - protein, 553 aa, Artificial Sequence
  Synthetic Construct
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVILIRLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVPAAATMVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 4 - DNA, 1662 bp, Artificial Sequence
  Synthetic Construct
atggactatggcggcgctttgtctgccgtcggacgcgaacttttgttcgttactaatcctgtggtggtgaacgggtccgtcctggtccctgaggatcaatgttactgtgccggatggattgaatctcgcggcacgaacggcgctcagaccgcgtcaaatgtcctgcagtggcttgcagcaggattcagcattttgctgctgatgttctatgcctaccaaacctggaaatctacatgcggctgggaggagatctatgtgtgcgccattgaaatggttaaggtgattctcgagttcttttttgagtttaagaatccctctatgctctaccttgccacaggacaccgggtgcagtggctgcgctatgcagagtggctgctcacttgtcctgtcatccttatccgcctgagcaacctcaccggcctgagcaacgactacagcaggagaaccatgggactccttgtctcagacatcgggactatcgtgtggggggctaccagcgccatggcaaccggctatgttaaagtcatcttcttttgtcttggattgtgctatggcgcgaacacattttttcacgccgccaaagcatatatcgagggttatcatactgtgccaaagggtcggtgccgccaggtcgtgaccggcatggcatggctgtttttcgtgagctggggtatgttcccaattctcttcattttggggcccgaaggttttggcgtcctgagcgtctatggctccaccgtaggtcacacgattattgatctgatgagtaaaaattgttgggggttgttgggacactacctgcgcgtcctgatccacgagcacatattgattcacggagatatccgcaaaaccaccaaactgaacatcggcggaacggagatcgaggtcgagactctcgtcgaagacgaagccgaggccggagccgtgccagcggccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 5 - protein, 361 aa, Artificial Sequence
  Synthetic Construct
MTMVSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWEEIYVATIEMIKFIIEYFHEFDEPAVIYSSNGNKTVWLRYAEWLLTCPVVLIHLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVNKGTGKYESS

SEQ ID NO: 6 - DNA, 1080 bp, Artificial Sequence
  Synthetic Construct
accatggtgagcagaagaccctggctgctggccctggccctggccgtggccctggccgccggcagcgccggcgccagcaccggcagcgacgccaccgtgcccgtggccacccaggacggccccgactacgtgttccacagagcccacgagagaatgctgttccagaccagctacaccctggagaacaacggcagcgtgatctgcatccccaacaacggccagtgcttctgcctggcctggctgaagagtaacggcaccaacgccgagaagctggccgccaacatcctgcagtggatcaccttcgccctgagcgccctgtgcctgatgttctacggctaccagacctggaagagtacctgcggctgggaggagatctacgtggccaccatcgagatgatcaagttcatcatagagtacttccacgagttcgacgagcccgccgtgatctacagcagcaacggcaacaagaccgtgtggctgagatacgccgagtggctgctgacctgccccgtggtcctgatccacctgagcaacctgaccggcctggccaacgactacaacaagagaaccatgggcctgctggtgagcgacatcggcaccatcgtgtggggcaccaccgccgccctgagcaagggctacgtgagagtgatcttcttcctgatgggcctgtgctacggcatctacaccttcttcaacgccgccaaggtgtacatcgaggcctaccacaccgtgcccaagggcagatgcagacaggtggtgaccggcatggcctggctgttcttcgtgagctggggcatgttccccatcctgttcatcctgggccccgagggcttcggcgtgctgagcgtgtacggcagcaccgtgggccacaccatcatcgacctgatgagcaagaactgctggggcctgctgggccactacctgagagtgctgatccacgagcacatcctgatccacggcgacatcagaaagaccaccaagctgaacatcggcggcaccgagatcgaggtggagaccctggtggaggacgaggccgaggccggcgccgtgaacaagggcaccggcaagtacgagagcagc

SEQ ID NO: 7 - protein, 452 aa, Artificial Sequence
  Synthetic Construct
MAELISSATRSLFAAGGINPWPNPYHHEDMGCGGMTPTGECFSTEWWCDPSYGLSDAGYGYCFVEATGGYLVVGVEKKQAWLHSRGTPGEKIGAQVCQWIAFSIAIALLTFYGFSAWKATCGWEEVYVCCVEVLFVTLEIFKEFSSPATVYLSTGNHAYCLRYFEWLLSCPVILIRLSNLSGLKNDYSKRTMGLIVSCVGMIVFGMAAGLATDWLKWLLYIVSCIYGGYMYFQAAKCYVEANHSVPKGHCRMVVKLMAYAYFASWGSYPILWAVGPEGLLKLSPYANSIGHSICDIIAKEFWTFLAHHLRIKIHEHILIHGDIRKTTKMEIGGEEVEVEEFVEEEDEDTVAAPVVAVSKGEEVIKEFMRFKVRMEGSMNGHEFEIEGEGEGRPYEGTQTAKLKVTKGGPLPFAWDILSPQFMYGSKAYVKHPADIPDYKKLSFPEGFKWERV

SEQ ID NO: 8 - DNA, 1356 bp, Artificial Sequence
  Synthetic Construct
atggctgagctgatcagcagcgccaccagatctctgtttgccgccggaggcatcaacccttggcctaacccctaccaccacgaggacatgggctgtggaggaatgacacctacaggcgagtgcttcagcaccgagtggtggtgtgacccttcttacggactgagcgacgccggatacggatattgcttcgtggaggccacaggcggctacctggtcgtgggagtggagaagaagcaggcttggctgcacagcagaggcacaccaggagaaaagatcggcgcccaggtctgccagtggattgctttcagcatcgccatcgccctgctgacattctacggcttcagcgcctggaaggccacttgcggttgggaggaggtctacgtctgttgcgtcgaggtgctgttcgtgaccctggagatcttcaaggagttcagcagccccgccacagtgtacctgtctaccggcaaccacgcctattgcctgcgctacttcgagtggctgctgtcttgccccgtgatcctgatcagactgagcaacctgagcggcctgaagaacgactacagcaagcggaccatgggcctgatcgtgtcttgcgtgggaatgatcgtgttcggcatggccgcaggactggctaccgattggctcaagtggctgctgtatatcgtgtcttgcatctacggcggctacatgtacttccaggccgccaagtgctacgtggaagccaaccacagcgtgcctaaaggccattgccgcatggtcgtgaagctgatggcctacgcttacttcgcctcttggggcagctacccaatcctctgggcagtgggaccagaaggactgctgaagctgagcccttacgccaacagcatcggccacagcatctgcgacatcatcgccaaggagttttggaccttcctggcccaccacctgaggatcaagatccacgagcacatcctgatccacggcgacatccggaagaccaccaagatggagatcggaggcgaggaggtggaagtggaagagttcgtggaggaggaggacgaggacacagtggcggcaccggtagtagcagtgagtaagggcgaggaagtgatcaaagagttcatgcggtttaaggtgagaatggaaggaagcatgaacggccacgagttcgaaattgagggagaaggagagggacggccctacgagggcacccagacagccaagctgaaagtgacaaagggcgggcctctgccattcgcttgggacatcctgagcccacagtttatgtacggctccaaggcctatgtgaaacatccagctgacattcccgattataagaaactgagcttccccgaggggtttaagtgggaaagagtg

SEQ ID NO: 9 - protein, 325 aa, Artificial Sequence
  Synthetic Construct
MTHAFISAVPSAEATIRGLLSAAAVVTPAADAHGETSNATTAGADHGCFPHINHGTELQHKIAVGLQWFTVIVAIVQLIFYGWHSFKATTGWEEVYVCVIELVKCFIELFHEVDSPATVYQTNGGAVIWLRYSMWLLTCPVILIHLSNLTGLHEEYSKRTMTILVTDIGNIVWGITAAFTKGPLKILFFMIGLFYGVTCFFQIAKVYIESYHTLPKGVCRKICKIMAYVFFCSWLMFPVMFIAGHEGLGLITPYTSGIGHLILDLISKNTWGFLGHHLRVKIHEHILIHGDIRKTTTINVAGENMEIETFVDEEEEGGVAAPVVA

SEQ ID NO: 10 - DNA, 975 bp, Artificial Sequence
  Synthetic Construct
atgacccacgcctttatctcagccgtgcctagcgccgaagccacaattagaggcctgctgagcgccgcagcagtggtgacaccagcagcagacgctcacggagaaacctctaacgccacaacagccggagccgatcacggttgcttcccccacatcaaccacggaaccgagctgcagcacaagatcgcagtgggactccagtggttcaccgtgatcgtggctatcgtgcagctcatcttctacggttggcacagcttcaaggccacaaccggctgggaggaggtctacgtctgcgtgatcgagctcgtcaagtgcttcatcgagctgttccacgaggtcgacagcccagccacagtgtaccagaccaacggaggagccgtgatttggctgcggtacagcatgtggctcctgacttgccccgtgatcctgatccacctgagcaacctgaccggactgcacgaagagtacagcaagcggaccatgaccatcctggtgaccgacatcggcaacatcgtgtgggggatcacagccgcctttacaaagggccccctgaagatcctgttcttcatgatcggcctgttctacggcgtgacttgcttcttccagatcgccaaggtgtatatcgagagctaccacaccctgcccaaaggcgtctgccggaagatttgcaagatcatggcctacgtcttcttctgctcttggctgatgttccccgtgatgttcatcgccggacacgagggactgggcctgatcacaccttacaccagcggaatcggccacctgatcctggatctgatcagcaagaacacttggggcttcctgggccaccacctgagagtgaagatccacgagcacatcctgatccacggcgacatccggaagacaaccaccatcaacgtggccggcgagaacatggagatcgagaccttcgtcgacgaggaggaggagggaggagtggcggcaccggtagtagca

SEQ ID NO: 11 - protein, 352 aa, Artificial Sequence
  Synthetic Construct
MSRLVAASWLLALLLCGITSTTTASSAPAASSTDGTAAAAVSHYAMNGFDELAKGAVVPEDHFVCGPADKCYCSAWLHSHGSKEEKTAFTVMQWIVFAVCIISLLFYAYQTWRATCGWEEVYVTIIELVHVCFGLWHEVDSPCTLYLSTGNMVLWLRYAEWLLTCPVILIHLSNLTGMKNDYNKRTMALLVSDVGCIVWGTTAALSTDFVKIIFFFLGLLYGFYTFYAAAKIYIEAYHTVPKGICRQLVRLQAYDFFFTWSMFPILFMVGPEGFGKITAYSSGIAHEVCDLLSKNLWGLMGHFIRVKIHEHILVHGNITKKTKVNVAGDMVELDTYVDQDEEHDEGAAPVVA

SEQ ID NO: 12 - DNA, 1056 bp, Artificial Sequence
  Synthetic Construct
atgagcagactggtcgccgcttcttggctgctggctctcctcctctgcggaattaccagcacaacaacagcctctagcgccccagcagcttcttctacagacggaacagccgccgcagcagtgtctcactacgccatgaacggcttcgacgagctggctaaaggagccgtggtgccagaagaccactttgtctgcggaccagccgacaagtgctattgctccgcttggctgcacagccacggaagcaaggaggagaagaccgccttcaccgtcatgcagtggatcgtgttcgccgtctgcatcatcagcctgctgttctacgcctaccagacttggagggctacttgcggttgggaggaggtgtacgtgaccatcatcgagctggtccacgtctgcttcggactctggcacgaggtcgatagcccttgtaccctgtacctgagcacaggcaacatggtcctctggctgagatacgccgagtggctgctgacttgccccgtgatcctgatccacctgagcaacctgaccggcatgaagaacgactacaacaagcggaccatggccctgctggtgtcagacgtgggctgtatcgtgtggggaacaacagccgccctgagcaccgatttcgtgaagatcatcttcttcttcctgggcctgctgtacggcttctacaccttctacgccgccgccaagatctacatcgaggcctaccacaccgtgcccaagggcatttgtagacagctcgtgcggctgcaggcctacgacttcttcttcacttggagcatgttccccatcctgttcatggtcggcccagagggattcggcaagatcaccgcctacagcagcggaatcgcccacgaagtgtgcgatctgctgagcaagaacctctggggcctgatgggccacttcatccgcgtgaagatccacgagcacatcctggtgcacggcaacatcaccaagaagaccaaggtcaacgtggccggcgacatggtggaactggacacctacgtggaccaggacgaggaacacgacgagggagcggcaccggtagtagca

SEQ ID NO: 13 - protein, 310 aa, Artificial Sequence
  Synthetic Construct
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTAPVILIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVP

SEQ ID NO: 14 - DNA, 930 bp, Artificial Sequence
  Synthetic Construct
atggactatggcggcgctttgtctgccgtcggacgcgaacttttgttcgttactaatcctgtggtggtgaacgggtccgtcctggtccctgaggatcaatgttactgtgccggatggattgaatctcgcggcacgaacggcgctcagaccgcgtcaaatgtcctgcagtggcttgcagcaggattcagcattttgctgctgatgttctatgcctaccaaacctggaaatctacatgcggctgggaggagatctatgtgtgcgccattgaaatggttaaggtgattctcgagttcttttttgagtttaagaatccctctatgctctaccttgccacaggacaccgggtgcagtggctgcgctatgcagagtggctgctcactgcccctgtcatccttatccacctgagcaacctcaccggcctgagcaacgactacagcaggagaaccatgggactccttgtctcagacatcgggactatcgtgtggggggctaccagcgccatggcaaccggctatgttaaagtcatcttcttttgtcttggattgtgctatggcgcgaacacattttttcacgccgccaaagcatatatcgagggttatcatactgtgccaaagggtcggtgccgccaggtcgtgaccggcatggcatggctgtttttcgtgagctggggtatgttcccaattctcttcattttggggcccgaaggttttggcgtcctgagcgtctatggctccaccgtaggtcacacgattattgatctgatgagtaaaaattgttgggggttgttgggacactacctgcgcgtcctgatccacgagcacatattgattcacggagatatccgcaaaaccaccaaactgaacatcggcggaacggagatcgaggtcgagactctcgtcgaagacgaagccgaggccggagccgtgcca

SEQ ID NO: 15 - protein, 310 aa, Artificial Sequence
  Synthetic Construct
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTSPVILIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVP

SEQ ID NO: 16 - DNA, 930 bp, Artificial Sequence
  Synthetic Construct
atggactatggcggcgctttgtctgccgtcggacgcgaacttttgttcgttactaatcctgtggtggtgaacgggtccgtcctggtccctgaggatcaatgttactgtgccggatggattgaatctcgcggcacgaacggcgctcagaccgcgtcaaatgtcctgcagtggcttgcagcaggattcagcattttgctgctgatgttctatgcctaccaaacctggaaatctacatgcggctgggaggagatctatgtgtgcgccattgaaatggttaaggtgattctcgagttcttttttgagtttaagaatccctctatgctctaccttgccacaggacaccgggtgcagtggctgcgctatgcagagtggctgctcacttctcctgtcatccttatccacctgagcaacctcaccggcctgagcaacgactacagcaggagaaccatgggactccttgtctcagacatcgggactatcgtgtggggggctaccagcgccatggcaaccggctatgttaaagtcatcttcttttgtcttggattgtgctatggcgcgaacacattttttcacgccgccaaagcatatatcgagggttatcatactgtgccaaagggtcggtgccgccaggtcgtgaccggcatggcatggctgtttttcgtgagctggggtatgttcccaattctcttcattttggggcccgaaggttttggcgtcctgagcgtctatggctccaccgtaggtcacacgattattgatctgatgagtaaaaattgttgggggttgttgggacactacctgcgcgtcctgatccacgagcacatattgattcacggagatatccgcaaaaccaccaaactgaacatcggcggaacggagatcgaggtcgagactctcgtcgaagacgaagccgaggccggagccgtgcca

SEQ ID NO: 17 - protein, 300 aa, Artificial Sequence
  Synthetic Construct
MDYPVARSLIVRYPTDLGNGTVCMPRGQCYCEGWLRSRGTSIEKTIAITLQWVVFALSVACLGWYAYQAWRATCGWEEVYVALIEMMKSIIEAFHEFDSPATLWLSSGNGVVWMRYGEWLLTCPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEED

SEQ ID NO: 18 - DNA, 900 bp, Artificial Sequence
  Synthetic Construct
atggactatcctgttgctagaagcctcatagttcgctacccaaccgacctcggaaacggcaccgtctgcatgccaagaggacagtgttactgtgaaggttggcttcggagtcgcggcacttccattgaaaagacaatagcaattactcttcagtgggtagtctttgctttgtcagtggcttgcctggggtggtatgcgtatcaagcgtggcgagctacctgcggatgggaggaggtttacgtagccttgatagaaatgatgaaaagcatcatcgaggccttccacgagttcgacagccctgcaacactgtggctgtcttcagggaacggcgtagtttggatgcggtatggcgaatggctcctcacctgcccggtccttctgatccatctgagcaacctcacaggcctgaaggacgattatagcaaaaggactatgggcctgttggtttctgatgtgggatgcatcgtgtggggcgcaaccagcgccatgtgtacggggtggacgaagatcctgttcttcctcatctcattgagctatggtatgtatacctattttcatgctgctaaagtttatatcgaagcattccacacagttccaaaagggatttgtcgagaactggtccgagtgatggcctggacattctttgtggcttggggaatgtttccagtcctgtttctgctgggcacggaaggattcggtcatatcagcccttatggatctgccattgggcactccatcctcgacctgattgcaaagaacatgtggggtgtgctggggaattacctgcgcgtcaaaatccacgagcacatcctgttgtatggcgacatcagaaagaagcagaaaattacgatcgccggccaagagatggaggttgagacactggtggctgaagaggaggac

SEQ ID NO: 19 - protein, 344 aa, Artificial Sequence
  Synthetic Construct
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWEEIYVATIEMIKFIIEYFHEFDEPAVIYSSNGNKTVWLRYAEWLLTCPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEED

SEQ ID NO: 20 - DNA, 1032 bp, Artificial Sequence
  Synthetic Construct
atgtcgcggaggccatggcttcttgccctagcgctggcagtggcgctggcggccggcagcgcaggagcctcgactggcagtgacgcgacggtgccggtcgcgactcaggatggccccgactacgttttccaccgtgcccacgagcgcatgctcttccaaacctcatacactcttgagaacaatggttctgttatttgcatcccgaacaacggccagtgcttctgcttggcttggcttaaatccaacggaacaaatgccgagaagttggctgccaacattctgcagtggattacttttgcgctttcagcgctctgcctgatgttctacggctaccagacctggaagtctacttgcggctgggaggagatttacgtggccacgatcgagatgatcaagttcatcatcgagtatttccatgagtttgacgaacctgcggtgatctactcatccaacggcaacaagaccgtgtggcttcgttacgcggagtggctgctcacctgcccggtccttctgatccatctgagcaacctcacaggcctgaaggacgattatagcaaaaggactatgggcctgttggtttctgatgtgggatgcatcgtgtggggcgcaaccagcgccatgtgtacggggtggacgaagatcctgttcttcctcatctcattgagctatggtatgtatacctattttcatgctgctaaagtttatatcgaagcattccacacagttccaaaagggatttgtcgagaactggtccgagtgatggcctggacattctttgtggcttggggaatgtttccagtcctgtttctgctgggcacggaaggattcggtcatatcagcccttatggatctgccattgggcactccatcctcgacctgattgcaaagaacatgtggggtgtgctggggaattacctgcgcgtcaaaatccacgagcacatcctgttgtatggcgacatcagaaagaagcagaaaattacgatcgccggccaagagatggaggttgagacactggtggctgaagaggaggac

SEQ ID NO: 21 - protein, 235 aa, Artificial Sequence
  Synthetic Construct
VSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 22 - DNA, 705 bp, Artificial Sequence
  Synthetic Construct
gtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaag

SEQ ID NO: 23 - DNA, 720 bp, Artificial Sequence
  Synthetic Construct
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 24 - DNA, 705 bp, Artificial Sequence
  Synthetic Construct
atggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtaa

SEQ ID NO: 25 - DNA, 602 bp, Artificial Sequence
  Synthetic Construct
tagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaat

SEQ ID NO: 26 - DNA, 1682 bp, Artificial Sequence
  Synthetic Construct
acgcgtaagctttcctttaggaacagaggcttcgagcctttaaggctgcgtacttgcttctcctaataccagagactcaaaaaaaaaaaaaaagttccagattgctggacaatgacccgggtctcatcccttgaccctgggaaccgggtccacattgaatcaggtgcgaatgttcgctcgccttctctgcctttcccgcctcccctcccccggccgcggccccggttccccccctgcgctgcaccctcagagttggctgcagccggcgagctgttcccgtcaatccctccctcctttacacaggatgtccatattaggacatctgcgtcagcaggtttccacggccggtccctgttgttctgggggggggaccatctccgaaatcctacacgcggaaggtctaggagaccccctaagatcccaaatgtgaacactcataggtgaaagatgtatgccaagacgggggttgaaagcctggggcgtagagttgacgacagagcgcccgcagagggccttggggcgcgcttccccccccttccagttccgcccagtgacgtaggaagtccatccattcacagcgcttctataaaggcgccagctgaggcgcctactactccaaccgcgactgcagcgagcaactgagaagactggatagagccggcggttccgcgaacgagcagtgaccgcgctcccacccagctctgctctgcagctcccaccagtgtctacccctggaccccttgccgggctttccccaaacttcgaccatgatgttctcgggtttcaacgccgactacgaggcgtcatcctcccgctgcagtagcgcctccccggccggggacagcctttcctactaccattccccagccgactccttctccagcatgggctctcctgtcaacacacaggtgagtttggctttgtgtagccgccaggtccgcgctgagggtcgccgtggaggagacactggggtgtgactcgcaggggcgggggggtcttcctttttcgctctggagggagactggcgcggtcagagcagccttagcctgggaacccaggacttgtctgagcgcgtgcacacttgtcatagtaagacttagtgaccccttcccgcgcggcaggtttattctgagtggcctgcctgcattcttctctcggccgacttgtttctgagatcagccggggccaacaagtctcgagcaaagagtcgctaactagagtttgggaggcggcaaaccgcggcaatcccccctcccggggcagcctggagcagggaggagggaggagggaggagggtgctgcgggcgggtgtgtaaggcagtttcattgataaaaagcgagttcattctggagactccggagcagcgcctgcgtcagcgcagacgtcagggatatttataacaaaccccctttcgagcgagtgatgccgaagggataacgggaacgcagcagtaggatggaggggaaaggctgcgctgcggaattcaagggaggatattgggagagcttttatctccgatgaggtgcatacaggaagacataagcagtctctgaccggaatgcttctctctccctgcttcatgcgacactagggccacttgctccacctgtgtctggaacctcctcgctcacctccgctttcctctttttgttttgtttcaggacttttgcgcagatctctc

SEQ ID NO: 27 - DNA, 1268 bp, Artificial Sequence
  Synthetic Construct
tgtatgcagctggacctaggagagaagcaggagaggaagatccagcacaaaaaatctgaagctaaaaacaggacacagagatgggggaagaaaagagggcagagtgaggcaaaaagagactgaagagatgagggtggccgccaggcactttagataggggagaggctttatttacctctgtttgttttttttttttttttttttttttttttgcgaggtagtcttgcttagtctccaggctggagtgcagtggcacaatctcagctcactgcaacttccacctcctgggttcaagcaattctcctgcctcagcctcccgagtagctgggactacaggcgcatgcaaccgcgcctggctaatttttgtatttttagtagaaacggggtttcaccacgttagccaggatggtctggatctcctgacctcgtgatctgcccgcctccgccttccaaagtgctgggattacaggggtgagccacagcgcctggtccctatttacttctgtcttctacctccaggagatcaaagacgctggccttcagacctgatcagactcccaggggcagccaccacatgtatgacagagaacagaggatgcctgtttttgccccaaagctggaaattcatcacaacctgaggcccaggatctgctctgtgccggtcctctgggcagtgtggggtgcagaatggggtgcctaggcctgagcgttgcctggagcctaggccgggggccgccctcgggcaggcgtgggtgagagccaagaccgcgtgggccgcggggtgctggtaggagtggttggagagacttgcgaaggcggctggggtgttcggatttccaataaagaaacagagtgatgctcctgtgtctgaccgggtttgtgagacattgaggctgtcttgggcttcactggcagtgtgggccttcgtacccgggctacaggggtgcggctctgcctgttactgtcgagtgggtcgggccgtgggtatgagcgcttgtgtgcgctggggccaggtcgtgggtgcccccacccttcccccatcctcctcccttccccactccaccctcgtcggtcccccacccgcgctcgtacgtgcgcctccgccggcagctcctgactcatcgggggctccgggtcacatgcgcccgcgcggccctataggcgcctcctccgcccgccgcccgggagccgcagccgccgccgccactgccactcccgctctctcagcgccgccgtcgccaccgccaccgccaccgccactaccaccg

SEQ ID NO: 28 - DNA, 1064 bp, Artificial Sequence
  Synthetic Construct
aagaaaaggtgaatggttgggatgcatactggaaggaaacaacggaaatctgaaaaggtgtaagaacctaaacaaatttgtttatcacagaaaataaatcacaaaacaactttgcgttctttggcaagtttctttatgttaaacaagaattgctttttgcatcacatagatcttctaaactctttgttgaagaggtccttggtagtctgtatctaagccagttccttacggaagtggcactgagcggagtagataaagataggaacttttgaagggtcataatctctgtgtgcaaaaaagaagccacagtagtctgaagagctgtgcaggttttagggtgacactgggttgggaaccttggagctaagtgtcccacacctggcaagccatgacatacatattttctgttcaggcagaaactgagctttacaaaagtgaaatgagaaaaaaaaaaaaaccaaaaaccaggcacgtatattgagaaccattcagtccttcttagaattgcctcatacctttctcatgcatctttattaaattcagatgcaaattaattttagaaaagtctaaataggtgtgtgttttatttttctgtttcctaattaaatagtggtataagcctggaaatgctctatatctattttcggaaatctatagctcttgtttaggtaaatatcaggtacttagctaattaaatgtctcttgtttataggaaagtgtcagctttcaggatgttatgtgtatggctcaataaaattacgtacaaagtgacagcgtactctcttttcatgggctgaccttgtcgtcaccatcacctgaaaatggctccaaacaaaaatgacctaagggttgaaacaagataagatcaaattgacgtcatggtaaaaattgacgtcatggtaattacaccaagtacccttcaatcattggatggaatttcctgttgatcccagggcttagatgcaggtggaaacactctgctggtataaaagcaggtgaggacttcattaactgcagttactgagaactcataagacgaagctaaaatccctcttcggatccac

SEQ ID NO: 29 - DNA, 937 bp, Artificial Sequence
  Synthetic Construct
ccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 30 - DNA, 1014 bp, Artificial Sequence
  Synthetic Construct
ccaattccttctcaaactcgaaagaaaccttctctagccccgtgggcgcgcggaggctgcgagcacaaacatcgccctcggccactgccagaaggccgggccccctgtccacacttggaaccccggggaacccttttgcttggcctcttgggtccagcggcccatccgtccaaggtccgggcggaggccgtccggaccctgctgctctctcggattcttgtttatttcccaaacaccacgcggagccactgcgcctccgcaacgatctcccccgcaccgccccggcgcgcccccgcccccacccaatcagcgcgcacaacttccccctcggctccggctcgcggattgaaccctcctgacatatttggggccattcttctcctttgttgctattttgctagcgacccgcgggtaatccccgcgcgggaggggggcgtgcattgtcgcgctgatggacgggcccatttggcggctccgcgccccccggaggagagacacaaagcccaggcacgtgcgcctccccatagagaagcagcagaccgtgaagggaggcggggccgggcgtgtgcctggaccgggcggggcggcggcgccgggcggggcgaccaggggcgcgcgcgggggccccgcgccctcaggtacatctgccgcacctaccgggcgacccccgagtcccggcccccttttggccgccccatcgccctcccaccctgccaggctgaggagctgcggacgcgctgattggctccaggggaagcgggaggcgagaacaatggccccctccccccgttaaaagggagcggctgccgggcccggggacagggacgcgcgtgcagggcgcagagctgggccgagccgtcgccggcgccacgcgagtcccgcagccgccgcgcccgggcaatgggccgggggcactgagggccgccggggccgagcgcggaggggggaccgagccagtgccgtgccctcgggccgcgccaacatgccccgcggcttcctggtgaagcgcagcaa

SEQ ID NO: 31 - DNA, 448 bp, Artificial Sequence
  Synthetic Construct
ctgcgctctcaggcacgacacgactcctccgctgcccaccgcagactgaggcagcgctgagtcgccggcgccgcagcgcagatggtcgcgcccgtgcccccctatctcgcgcctcgcgtggtgcggtccggctgggccggcggcggcgcggacgcgaccaaggtggccgggaaggggagtttgcgggggaccggcgagtgacgtcagcgcgccttcagtgctgaggcggcggtggcgcgcgccgccaggcgggggcgaaggcactgtccgcggtgctgaagctggcagtgcgcacgcgcctcgccgcatcctgtttcccctccccctctctgataggggatgcgcaatttggggaatgggggttgggtgcttgtccagtgggtcggggtcggtcgtcaggtaggcacccccaccccgcctcatcctggtcctaaaacccacttgcact

SEQ ID NO: 32 - DNA, 1293 bp, Artificial Sequence
  Synthetic Construct
ttaacattatggccttaggtcacttcatctccatggggttcttcttctgattttctagaaaatgagatgggggtgcagagagcttcctcagtgacctgcccagggtcacatcagaaatgtcagagctagaacttgaactcagattactaatcttaaattccatgccttgggggcatgcaagtacgatatacagaaggagtgaactcattagggcagatgaccaatgagtttaggaaagaagagtccagggcagggtacatctacaccacccgcccagccctgggtgagtccagccacgttcacctcattatagttgcctctctccagtcctaccttgacgggaagcacaagcagaaactgggacaggagccccaggagaccaaatcttcatggtccctctgggaggatgggtggggagagctgtggcagaggcctcaggaggggccctgctgctcagtggtgacagataggggtgagaaagcagacagagtcattccgtcagcattctgggtctgtttggtacttcttctcacgctaaggtggcggtgtgatatgcacaatggctaaaaagcagggagagctggaaagaaacaaggacagagacagaggccaagtcaaccagaccaattcccagaggaagcaaagaaaccattacagagactacaagggggaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagc

SEQ ID NO: 33 - protein, 185 aa, Artificial Sequence
  Synthetic Construct
MGVKVLFALICIAVAEAKPTENNEDFNIVAVASNFATTDLDADRGKLPGKKLPLEVLKEMEANARKAGCTRGCLICLSHIKCTPKMKKFIPGRCHTYEGDKESAQGGIGEAIVDIPEIPGFKDLEPMEQFIAQVDLCVDCTTGCLKGLANVQCSDLLKKWLPQRCATFASKIQGQVDKIKGAGGD

SEQ ID NO: 34 - DNA, 555 bp, Artificial Sequence
  Synthetic Construct
atgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgac

SEQ ID NO: 35 - protein, 250 aa, Artificial Sequence
  Synthetic Construct
MASKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKIIFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNYNAYLRASDDL

SEQ ID NO: 36 - DNA, 752 bp, Artificial Sequence
  Synthetic Construct
atggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcc

SEQ ID NO: 37 - protein, 185 aa, Artificial Sequence
  Synthetic Construct
MGVKVLFALICIAVAEAKPTENNEDFNIVAVASNFATTDLDADRGKLPGEKLPLEVLKELEANARKAGCTRGCLICLSHIKCTPKMKKFIPGRCHTYEGDKESAQGGIGEAIDDIPEIPGFKDLEPIEQFIAQVDLCVDCTTGCLKGLANVQCSDLLKKWLPQRCATFASKIQGQVDKIKGAGDD

SEQ ID NO: 38 - DNA, 555 bp, Artificial Sequence
  Synthetic Construct
atgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcgagaagctgccgctggaggtgctcaaagagctggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgacgacattcctgagattcctgggttcaaggacttggagcccatcgagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtgatgac

SEQ ID NO: 39 - protein, 185 aa, Artificial Sequence
  Synthetic Construct
MGVKVLFALICIAVAEAKPTENNEDFNIVAVASNFATTDLDADRGKLPGKKLPLEVLKELEANARKAGCTRGCLICLSHIKCTPKMKKFIPGRCHTYEGDKESAQGGIGEAIVDIPEIPGFKDLEPLEQFIAQVDLCVDCTTGCLKGLANVQCSDLLKKWLPQRCATFASKIQGQVDKIKGAGGD

SEQ ID NO: 40 - DNA, 555 bp, Artificial Sequence
  Synthetic Construct
atgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagctggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccctggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgac

SEQ ID NO: 41 - protein, 171 aa, Artificial Sequence
  Synthetic Construct
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 42 - DNA, 513 bp, Artificial Sequence
  Synthetic Construct
atggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcg

SEQ ID NO: 43 - DNA, 54 bp, Artificial Sequence
  Synthetic Construct
gagggcaggggaagtcttctaacatgcggggacgtggaggaaaatcccggcccc

SEQ ID NO: 44 - DNA, 72 bp, Artificial Sequence
  Synthetic Construct
ggatcaggcagcggcgccacgaacttctctctgttaaagcaagcaggagacgtggaagaaaaccccggtccc

SEQ ID NO: 45 - DNA, 111 bp, Artificial Sequence
  Synthetic Construct
atgcgaggcgacggagagccgagtggagttcctgtagctgtggtgctgctgccagtgtttgcccttaccctggtagcagtttgggccttcgtgagataccgaaagcagctc

SEQ ID NO: 46 - DNA, 121 bp, Artificial Sequence
  Synthetic Construct
atggtgggccggaacagcgccatcgccgcggggctgtgcggtgccctcttcatagggtactgcatctactttgaccgcaaaaggcgaggtgaccccaacttcaaggggctagcgctaccgg

SEQ ID NO: 47 - DNA, 420 bp, Artificial Sequence
  Synthetic Construct
atgcgcgccccttctgctagggcgctactgctgattccgcgtcggggccctgccgtgcgagcgtgggccccggccgtctcctctcggatatggctggcttctgaatggaccccgctcgtacgcgcgtggacctctctgatccacaagccgggttcgggcctccgctttcccgcgcccctatccgggctgcctggcggcgtggggcagtgggccacctcctcgggggcccgcaggtgctgggtgctggcaggaccccgcgccgcacatcccctgttcgccaggctccagggtgcagctgccaccggtgtgcgagaccttgggaacgactcgcagcggcgtcccgcggcgaccgggcgctcagaagtatggaagctcctagggctggtgcgccccgagcgcgggagactgtcagctgcagtt

SEQ ID NO: 48 - DNA, 315 bp, Artificial Sequence
  Synthetic Construct
atgcgcgccccttctgctagggcgctactgctgattccgcgtcggggccctgccgtgcgagcgtgggccccggccgtctcctctcggatatggctggcttctgaatggaccccgctcgtacgcgcgtggacctctctgatccacaagccgggttcgggcctccgctttcccgcgcccctatccgggctgcctggcggcgtggggcagtgggccacctcctcgggggcccgcaggtgctgggtgctggcaggaccccgcgccgcacatcccctgttcgccaggctccagggtgcagctgccaccggtgtgcgagac

SEQ ID NO: 49 - DNA, 87 bp, Artificial Sequence
  Synthetic Construct
atgtccgtcctgacgccgctgctgctgcggggcttgacaggctcggcccggcggctcccagtgccgcgcgccaagatccattcgttg

SEQ ID NO: 50 - DNA, 349 bp, Artificial Sequence
  Synthetic Construct
atgaatgcttccagtcggaatgtgtttgacacgttgatcagggtgttgacagaaagtatgttcaaacatcttcggaaatgggtcgtcactcgcttttttgggcattctcggcaaagagcaaggctagtctccaaagatggaaggtgcaacatagaatttggcaatgtggaggcacagtcaaggtttatattctttgtggacatctggacaacggtacttgacctcaagtggagatacaaaatgaccattttcatcacagccttcttggggagttggtttttctttggtctcctgtggtatgcagtagcgtacattcacaaagacctcccggaattccatccttctgcca

SEQ ID NO: 51 - DNA, 44 bp, Artificial Sequence
  Synthetic Construct
actcactatagggagacccaagcttgccaccatgcgcgcccctt

SEQ ID NO: 52 - DNA, 32 bp, Artificial Sequence
  Synthetic Construct
ttcccagcataactgcagctgacagtctcccg

SEQ ID NO: 53 - DNA, 28 bp, Artificial Sequence
  Synthetic Construct
agctgcagttatgctgggaaacggcagc

SEQ ID NO: 54 - DNA, 46 bp, Artificial Sequence
  Synthetic Construct
tatcctcctcgcccttgctcacggatcctgctactaccggtgccgc

SEQ ID NO: 55 - DNA, 25 bp, Artificial Sequence
  Synthetic Construct
caccgaattcacattgattattgag

SEQ ID NO: 56 - DNA, 39 bp, Artificial Sequence
  Synthetic Construct
tttttatttctagactacacctcgttctcgtagcagaac

SEQ ID NO: 57 - DNA, 16 bp, Artificial Sequence
  Synthetic Construct
gtaaaacgacggccag

SEQ ID NO: 58 - DNA, 17 bp, Artificial Sequence
  Synthetic Construct
caggaaacagctatgac

SEQ ID NO: 59 - DNA, 24 bp, Artificial Sequence
  Synthetic Construct
gactttgaccgtttacgtggagac

SEQ ID NO: 60 - DNA, 24 bp, Artificial Sequence
  Synthetic Construct
ccttaagccacgcccacacatttc